(19) United States
(12) Patent Application Publication
(10) Pub. No.: US 2019/0206560 A1
(43) Pub. Date: Jul. 4, 2019

(54) NOTE INFORMATION MANAGEMENT DEVICE FOR MEDICAL INSTRUMENTS AND NOTE INFORMATION MANAGEMENT SYSTEM FOR MEDICAL INSTRUMENTS

(71) Applicant: Roland DG Corporation, Hamamatsu-shi, Shizuoka (JP)

(72) Inventors: Masaki HANAJIMA, Hamamatsu-shi (JP); Takeshi TSUJI, Hamamatsu-shi (JP); Takaaki KOKUBO, Hamamatsu-shi (JP)

(21) Appl. No.: 16/322,796

(22) PCT Filed: Aug. 4, 2017
(86) PCT No.: PCT/JP2017/028430

§ 371 Date: Feb. 1, 2019

(30) Foreign Application Priority Data
Aug. 4, 2016 (JP) 2016-153785
Aug. 24, 2016 (JP) 2016-163558
Aug. 29, 2016 (JP) 2016-166792
Aug. 29, 2016 (JP) 2016-166795
Aug. 30, 2016 (JP) 2016-167633

(51) Int. Cl. G16H 40/60; G06T 7/00

(52) U.S. Cl. CPC G16H 40/60; G06T 2207/30004; G06T 7/0012

(57) ABSTRACT

A note information management device for medical instruments includes an original data storage that stores original image data for one or more medical instruments, a note data generator that generates one or more note data, each of the note data representing a note information entered for a predetermined one of the image data, a note data storage that chronologically stores the generated noted data with association to the predetermined image data, and a display controller that allows a note data selected from the note data that are associated with the predetermined image data to be displayed on the predetermined original image data.

### BACKGROUND OF THE INVENTION

### 1. Field of the Invention

The invention according to the present preferred embodiments relates to note information management devices for medical instruments and note information management systems for medical instruments including such a device.

### 2. Description of the Related Art

There are many similar models of medical instruments used for surgeries and examinations in patients. For example, surgical forceps have handles with the same shape but have tips with different shapes depending on their purpose or regions where they are used. As a result, it is difficult for those who are not familiar with these instruments to distinguish them.

Consequently, errors can occur, such as confusion of instrument choice or the use of a wrong procedure for cleaning or sterilization (e.g., cleaning or sterilization of an instrument using a procedure that is different from what should be used) during a step or steps in collecting a used medical instrument; in cleaning, assembling, sterilizing, or storing a medical instrument; or in delivering a medical instrument for surgery.

Furthermore, medical instruments may be managed as a set of instruments, and the contents frequently vary because of, for example, failure, repair, omission of one or more instruments, or assignment of substitutions. Furthermore, confusion of instrument choice or the use of a wrong procedure is more likely to occur in sets including multiple instruments.

To avoid the aforementioned confusion of instrument choice or to know the contents that often vary, note information such as directions is added in the actual medical field.

By way of example, medical instruments are managed using an operation procedure written on a sheet of paper by directly writing note information on or attaching a sheet of paper with note information to the photograph of a medical instrument.

In addition, there are medical instrument management systems having a function of referring information registered in a database and generating a management report associated with the medical instruments.

However, there are various problems when managing note information on a paper basis. For example, multiple pieces of note information attached randomly may poses a problem that it is not known which note information is the one that should be checked, or which could be the latest note information. Even when pieces of note information are recorded in chronological order, a history about unnecessary note information may be left, or previous note information may be discarded for reasons of storage space or entry space. In addition, there are cases where the paper on which the note information is written is lost. Therefore, it cannot be said that, in the paper-based method, the note information is adequately managed. For a set of medical instruments where note information is updated frequently as the content thereof changes, its management tends to be particularly insufficient.

Furthermore, in the medical instrument management system described in Medical Instrument Management System “CEIA System,” [online], ARCADIA SYSTEMS Inc., [retrieved on Jun. 24, 2016] on the Internet <URL: http://www.arc-mec.com/main.cgi?c=4:2>, image data and management reports are displayed on separate screens. Accordingly, even when the directions and contents of a set are described in a management report, it is difficult to understand the content. In other words, even when the medical instrument management systems described in the Medical Instrument Management System “CEIA System,” [online], ARCADIA SYSTEMS Inc., [retrieved on Jun. 24, 2016] on the Internet <URL: http://www.arc-mec.com/main.gi?c=4:2> are used, it is difficult to adequately manage note information for medical instruments.

### SUMMARY OF THE INVENTION

Preferred embodiments of the present invention provide note information management devices for medical instruments and note information management systems for medical instruments to manage note information for medical instruments.

According to an example embodiment of the present invention, a note information management device for medical instruments includes an original data storage that stores original image data for one or more medical instruments; a note data generator that generates one or more note data, each of the note data representing a note information entered for a predetermined one of the image data; a note data storage that chronologically stores the generated noted data with association to the predetermined image data; and a display controller that allows a note data selected from the note data that are associated with the predetermined image data to be displayed on the predetermined original image data.

According to preferred embodiments of the present invention, it is possible to manage note information for medical instruments.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a diagrammatic representation showing a configuration of a note information management system for medical instruments according to a first preferred embodiment of the present invention.

FIG. 2 is a diagram showing an exemplified hardware configuration of the note information management device for medical instruments according to the first preferred embodiment of the present invention.

FIG. 3 is a diagram showing an exemplified software configuration of the note information management device for medical instruments according to the first preferred embodiment of the present invention.

FIG. 4 is a diagram showing an example of data stored in an original data storage according to the first preferred embodiment of the present invention.

FIG. 5A is a diagram showing a display screen on a terminal in the first preferred embodiment of the present invention.

FIG. 5B is a diagram showing a display screen on the terminal in the first preferred embodiment of the present invention.

FIG. 5C is a diagram showing a display screen on the terminal in the first preferred embodiment of the present invention.

FIG. 5D is a diagram showing a display screen on the terminal in the first preferred embodiment of the present invention.

FIG. 6 is a diagram showing an example of data stored in a note data storage according to the first preferred embodiment of the present invention.

FIG. 7 is a diagram showing an exemplified hardware configuration of a server according to the first preferred embodiment of the present invention.

FIG. 8 is a diagram showing an exemplified software configuration of the server according to the first preferred embodiment of the present invention.

FIG. 9 is a flow chart illustrating a processing in the note information management system for medical instruments according to the first preferred embodiment of the present invention.

FIG. 10 is a diagrammatic representation showing a configuration of a computer-aided management system for medical instruments according to a second preferred embodiment of the present invention.

FIG. 11 is a diagram showing an example of data stored in a medical procedure storage according to the second preferred embodiment of the present invention.

FIG. 12 is a diagram showing an example of data stored in a medical instrument storage according to the second preferred embodiment of the present invention.

FIG. 13 is a diagram showing an example of data stored in a to-be-used instrument storage according to the second preferred embodiment of the present invention.

FIG. 14 shows a screen displayed on a display of a terminal according to the second preferred embodiment of the present invention.

FIG. 15 shows a screen displayed on the display of the terminal according to the second preferred embodiment of the present invention.

FIG. 16A shows a screen displayed on the display of the terminal when counting of instruments is performed in the second preferred embodiment of the present invention.

FIG. 16B shows a screen displayed on the display of the terminal when counting of instruments is performed in the second preferred embodiment of the present invention.

FIG. 16C shows a screen displayed on the display of the terminal when counting of instruments is performed in the second preferred embodiment of the present invention.

FIG. 17A shows a screen displayed on the display of the terminal when counting of instruments is performed in the second preferred embodiment of the present invention.

FIG. 17B shows a screen displayed on the display of the terminal when counting of instruments is performed in the second preferred embodiment of the present invention.

FIG. 17C shows a screen displayed on the display of the terminal when counting of instruments is performed in the second preferred embodiment of the present invention.

FIG. 17D shows a screen displayed on the display of the terminal when counting of instruments is performed in the second preferred embodiment of the present invention.

FIG. 18 is a diagram showing an example of data stored in the to-be-used instrument storage according to the second preferred embodiment of the present invention.

FIG. 19A shows a screen displayed on the display of the terminal when a medical instrument is deleted in the second preferred embodiment of the present invention.

FIG. 19B shows a screen displayed on the display of the terminal when a medical instrument is deleted in the second preferred embodiment of the present invention.

FIG. 19C shows a screen displayed on the display of the terminal when a medical instrument is deleted in the second preferred embodiment of the present invention.

FIG. 20A shows a screen displayed on the display of the terminal when a medical instrument is added in the second preferred embodiment of the present invention.

FIG. 20B shows a screen displayed on the display of the terminal when a medical instrument is added in the second preferred embodiment of the present invention.

FIG. 20C shows a screen displayed on the display of the terminal when a medical instrument is added in the second preferred embodiment of the present invention.

FIG. 20D shows a screen displayed on the display of the terminal when a medical instrument is added in the second preferred embodiment of the present invention.

FIG. 21A shows a screen displayed on the display of the terminal when a medical instrument is added in the second preferred embodiment of the present invention.

FIG. 21B shows a screen displayed on the display of the terminal when a medical instrument is added in the second preferred embodiment of the present invention.

FIG. 21C shows a screen displayed on the display of the terminal when a medical instrument is added in the second preferred embodiment of the present invention.

FIG. 21D shows a screen displayed on the display of the terminal when a medical instrument is added in the second preferred embodiment of the present invention.

FIG. 22A shows a screen displayed on the display of the terminal when a medical instrument is added in the second preferred embodiment of the present invention.

FIG. 22B shows a screen displayed on the display of the terminal when a medical instrument is added in the second preferred embodiment of the present invention.

FIG. 22C shows a screen displayed on the display of the terminal when a medical instrument is added in the second preferred embodiment of the present invention.

FIG. 22D shows a screen displayed on the display of the terminal when a medical instrument is added in the second preferred embodiment of the present invention.

FIG. 23 is a flow chart illustrating a processing in the computer-aided management system for medical instruments according to the second preferred embodiment of the present invention.

FIG. 24 is a flow chart illustrating a processing in the computer-aided management system for medical instruments according to the second preferred embodiment of the present invention.

FIG. 25 is a diagrammatic representation showing a configuration of a computer-aided management system for medical instruments according to a third preferred embodiment of the present invention.

FIG. 26 is a diagram showing a configuration of a network system.

FIG. 27 is a diagram showing a configuration of a server system.

FIG. 28 is a diagram schematically showing a data configuration of a classification table.

FIG. 29 is a diagram schematically showing a data configuration of a set information table.

FIG. 30 is a schematic representation of stored data of the transfer number of times and the non-use number of times which are summed up for each medical instrument.

FIG. 31 is a schematic representation of stored data of the total number of transfers and the total number of non-uses which are summed up for each item category.

FIG. 32 is a schematic representation of stored data of the transfer number of times and the non-use number of times which are summed up for each set and the total number of transfers and the total number of non-uses which are summed up for each set and item category.

FIG. 33 is a diagram showing a configuration of number-of-transfers log data.

FIG. 34 is a diagram showing a configuration of number-of-non-uses log data.

FIG. 35 is a diagram showing a configuration of a client system.

FIG. 36 is a flow chart illustrating a flow of processing in a management system at a time of transferring a set.

FIG. 37 is a diagram showing a display screen at a time of transferring a set.

FIG. 38 is a flow chart illustrating a flow of processing in a management system after a medical procedure.

FIG. 39 is a diagram showing a display screen after a medical procedure.

FIG. 40 is a diagram showing a display screen at a time of tallying.

FIG. 41 is a diagram showing a configuration of a network system.

FIG. 42 is a diagram showing a configuration of a server system.

FIG. 43 is a diagram schematically showing a data configuration of a classification table.

FIG. 44 is a diagram schematically showing a data configuration of a set information table.

FIG. 45 is a schematic representation of stored data of the transfer number of times and the non-use number of times which are summed up for each medical instrument.

FIG. 46 is a schematic representation of stored data of the total number of transfers and the total number of non-uses which are summed up for each item category.

FIG. 47 is a schematic representation of stored data of the transfer number of times and the non-use number of times which are summed up for each set and the total number of transfers and the total number of non-uses which are summed up for each set and item category.

FIG. 48 is a diagram showing a configuration of number-of-transfers log data.

FIG. 49 is a diagram showing a configuration of number-of-non-uses log data.

FIG. 50 is a diagram showing a configuration of a client system.

FIG. 51 is a flow chart illustrating a flow of processing in a management system at a time of transferring a set.

FIG. 52 is a diagram showing a display screen at a time of transferring a set.

FIG. 53 is a flow chart illustrating a flow of processing in a management system after a medical procedure.

FIG. 54 is a diagram showing a display screen after a medical procedure.

FIG. 55 is a diagram showing a display screen at a time of tallying.

FIG. 56 is a diagrammatic representation showing a configuration of an implant management system according a sixth preferred embodiment of the present invention.

FIG. 57 is a diagram showing an example of data stored in a borrowed instrument storage according to the sixth preferred embodiment of the present invention.

FIG. 58A shows a screen displayed on a display of a terminal when a borrowed instrument is registered in the sixth preferred embodiment of the present invention.

FIG. 58B shows a screen displayed on the display of the terminal when a borrowed instrument is registered in the sixth preferred embodiment of the present invention.

FIG. 58C shows a screen displayed on the display of the terminal when a borrowed instrument is registered in the sixth preferred embodiment of the present invention.

FIG. 58D shows a screen displayed on the display of the terminal when a borrowed instrument is registered in the sixth preferred embodiment of the present invention.

FIG. 59A shows a screen displayed on the display of the terminal when an instrument is registered in the sixth preferred embodiment of the present invention.

FIG. 59B shows a screen displayed on the display of the terminal when an instrument is registered in the sixth preferred embodiment of the present invention.

FIG. 60A shows a screen displayed on the display of the terminal when an implant is registered in the sixth preferred embodiment of the present invention.

FIG. 60B shows a screen displayed on the display of the terminal when an implant is registered in the sixth preferred embodiment of the present invention.

FIG. 61A shows a screen displayed on the display of the terminal when an item is registered in the sixth preferred embodiment of the present invention.

FIG. 61B shows a screen displayed on the display of the terminal when an item is registered in the sixth preferred embodiment of the present invention.

FIG. 62A shows a screen displayed on the display of the terminal after a borrowed instrument has been registered in the sixth preferred embodiment of the present invention.

FIG. 62B shows a screen displayed on the display of the terminal after a borrowed instrument has been registered in the sixth preferred embodiment of the present invention.

FIG. 63A shows a screen displayed on the display of the terminal when counting of instruments is performed in the sixth preferred embodiment of the present invention.

FIG. 63B shows a screen displayed on the display of the terminal when counting of instruments is performed in the sixth preferred embodiment of the present invention.

FIG. 64A shows a screen displayed on the display of the terminal when counting of items is performed in the sixth preferred embodiment of the present invention.

FIG. 64B shows a screen displayed on the display of the terminal when counting of items is performed in the sixth preferred embodiment of the present invention.

FIG. 65A shows a screen displayed on the display of the terminal when a borrowed instrument is cleaned, assembled, sterilized in the sixth preferred embodiment of the present invention.

FIG. 65B shows a screen displayed on the display of the terminal when a borrowed instrument is cleaned, assembled, sterilized in the sixth preferred embodiment of the present invention.

FIG. 66A shows a screen displayed on the display of the terminal when a borrowed instrument is returned in the sixth preferred embodiment of the present invention.

FIG. 66B shows a screen displayed on the display of the terminal when a borrowed instrument is returned in the sixth preferred embodiment of the present invention.

FIG. 67 a diagrammatic representation showing a configuration of an implant management system according to a seventh preferred embodiment of the present invention.

### DETAILED DESCRIPTION OF THE PREFERRED EMBODIMENTS

In the descriptions of the specification and the drawings that follow, at least the following features are disclosed with respect to various preferred embodiments of the present invention.

A note information management device for medical instruments in which a latest note data among the note data is allowed to be displayed thereon by the display controller is disclosed. With such a device, it is possible to provide the latest note information to an operator.

Furthermore, a note information management device for medical instruments in which only the predetermined original image data is displayed by the display controller is disclosed. With such a device, it is possible to correctly know the current status of each medical instrument.

Furthermore, a note information management device for medical instruments in which, when a new note information is entered with the note data being displayed on the predetermined image data, a new note data in which the new note information is merged with the note data is generated by the note data generator, and in which the new note data is stored in the note data storage as a data that is different from the note data is disclosed. With such a device, even when two or more pieces of note information are entered for a single image data, it is possible to check any note information at any time point according to a log about note information that are chronologically stored.

Furthermore, a note information management device for medical instruments in which the note data is a stroke data in which the note information is drawn on the predetermined image data is disclosed. The use of a stroke data in which a shape is drawn as a note data makes it possible for operators to easily visually understand directions.

Furthermore, a note information management device for medical instruments in which the medical instrument is a set including a plurality of instruments is disclosed. With this device, even when a medical instrument is a set of medical instruments, it is possible to enter note information for one or more instruments thereof.

Furthermore, a note information management system for medical instruments includes a plurality of note information management devices for medical instruments each including an original data storage that stores original image data for one or more medical instruments; a note data generator that generates one or more note data, each of the note data representing a note information entered for a predetermined one of the image data; a note data storage that chronologically stores the generated noted data with association to the predetermined image data; and a display controller that allows a note data selected from the note data that are associated with the predetermined image data to be displayed on the original data of the predetermined image data; and a server including a server-side original data storage that stores image data that are identical to the original image data stored in the original data storage; a server-side note data storage that chronologically stores the note data that are transmitted from one of the note information management devices for medical instruments with association to the predetermined image data; and a note data distributor that distributes the note data stored in the server-side note data storage to the other note information management device for medical instruments is disclosed. With such a system, note information for medical instruments is able to be managed.

In addition, also disclosed is a note information management system for medical instruments in which, when the image data is allowed to be displayed, the display controller of the other note information management device for medical instruments makes an inquiry to the server whether there is a note data that is not stored in the note data storage, and if it is determined by the note data distributor, based on the inquiry, that a note data that is not stored in the note data storage is stored in the server-side note data storage, the note data is distributed to the other note information management device for medical instruments by the note data distributor. With such a system, when an image data is displayed, it is possible to get note information entered for that image data without fail.

Furthermore, a note information management system for medical instruments in which the original data storage of one of the note information management devices for medical instruments and the original data storage of the other note information management device for medical instruments store a same image data for a medical instrument is disclosed. The aforementioned configuration in which the devices store the equal image data makes it possible to share the note information that has been entered with a certain device with all devices in the system.

### First Preferred Embodiment

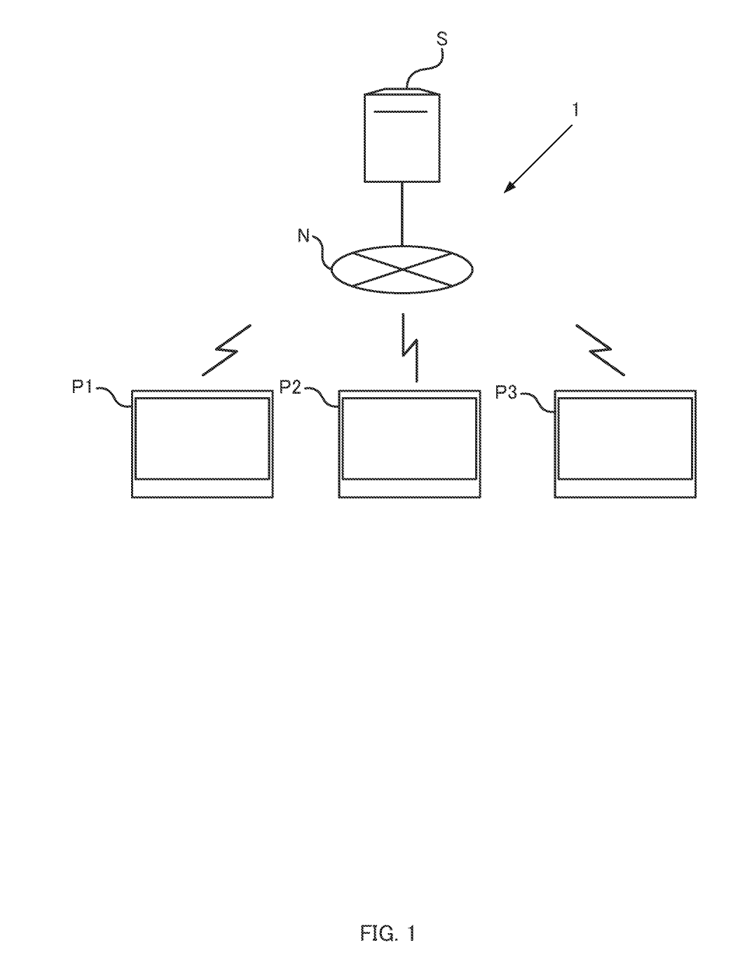

Referring to FIGS. 1 to 9, a note information management system for medical instruments according to this preferred embodiment (hereinafter, also referred to as a “system 1”) is described. It should be noted that an “image” and an “image data” correspond to each other one by one, and “note information” and “note data” correspond to each other one by one. Accordingly, they may be equated with each other in the present preferred embodiment.

Medical instruments are, for example, instruments used for surgery such as pairs of surgical forceps, scalpels, and rigid endoscopes and instruments used for examinations such as pairs of biopsy forceps, ultrasonic probes, and upper and lower endoscopes. Medical instruments in this preferred embodiment include a set of instruments (such as a surgical kit including pairs of forceps, scalpels, and pairs of scissors). Each medical instrument herein is assigned with a unique ID for identification.

As shown in FIG. 1, the system 1 includes a plurality of terminals (in this example, three terminals: a “terminal P1,” a “terminal P2,” and a “terminal P3”) and a server S. The terminals P1 to P3 can communicate with the server S via a network N.

The network N is, for example, a leased-line network in a hospital facility or the Internet.

The terminals P1 to P3 are devices for entering and displaying various kinds of information associated with medical instruments and exchanging information with the server S. Each terminal preferably is a desktop personal computer or a mobile terminal (such as a laptop PC and a tablet computer). The terminals can be located at different areas (such as an area where used medical instruments are collected, an area where medical instruments are cleaned, an area where medical instruments are assembled, an area where medical instruments are sterilized, and an area where medical instruments are stored) in a hospital facility.

The terminals herein define and function as devices for the management of note information (details are described later) entered for an image data for one or more medical instruments. The term “management (of note information)” used herein includes various kinds of processing performed on note information, such as storage, update, display, and distribution of note information. The terminals P1 to P3 are examples of a “note information management device for medical instruments.”

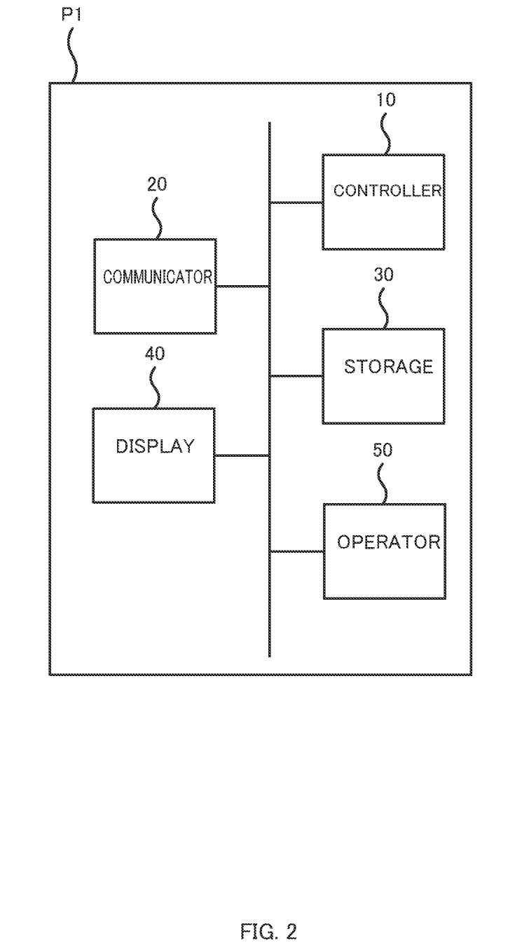

Since these terminals have similar hardware configurations, the description is made using the terminal P1 as an example. As shown in FIG. 2, the terminal P1 preferably includes a controller 10, a communicator 20, a storage 30, a display 40, and an operator 50.

The controller 10 preferably includes a CPU and a memory (which are not shown). The CPU achieves different kinds of control functions by executing an operating program or programs stored in the memory. The memory is a storage that stores a program or programs executed by the CPU or temporarily stores various pieces of information upon execution of the program(s).

The communicator 20 provides an interface for the communication between the terminal P1 and the server S. The storage 30 is a large-capacity storage that stores various kinds of data. The display 40 is a display to allow images for medical instruments and note information to be displayed. The operator 50 is a structure with which an operator enters instructions to the terminal P1. The operator 50 is, for example, an input interface or device such as a mouse. Alternatively, the display 40 of a touch-panel screen type may double as the operator 50. The controller 10 detects an operation signal from the operator 50 and executes a corresponding processing.

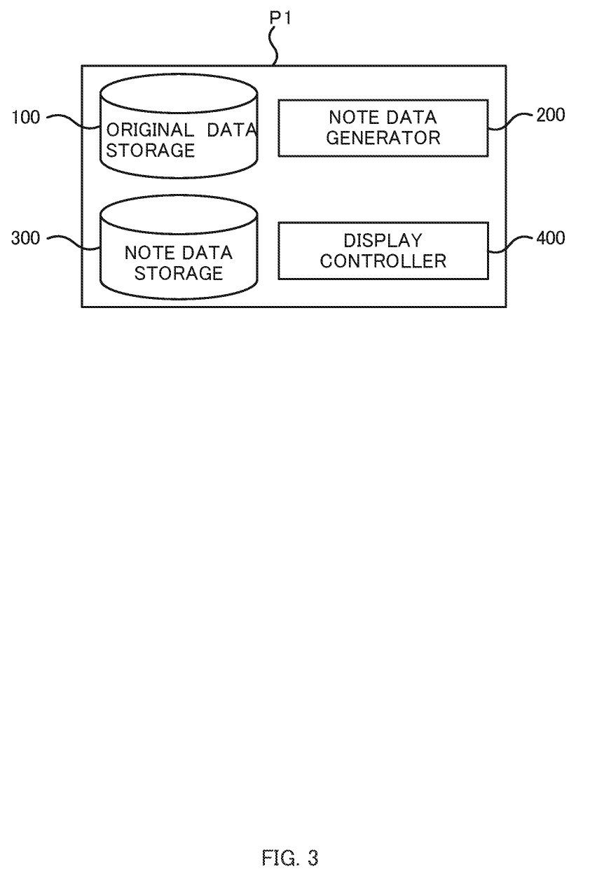

FIG. 3 is a diagram showing an exemplified software configuration in the terminal P1. The terminal P1 preferably includes an original data storage 100, a note data generator 200, a note data storage 300, and a display controller 400. The original data storage 100 and the note data storage 300 are portions of a storage region of the storage 30. The note data generator 200 and the display controller 400 are achieved when the CPU of the controller 10 executes a program or programs stored in the memory.

The original data storage 100 stores original image data for medical instruments. Each image data for one or more medical instruments is a data to display an image of the medical instrument(s) on the display 40. Each image data is a photographed data itself obtained by taking a picture of one or more medical instruments beforehand or a CG data representing the shape(s) of one or more medical instruments using a computer graphics. This original image data does not contain data for things other than an image of the medical instrument(s) (i.e., data that does not contain any note data).

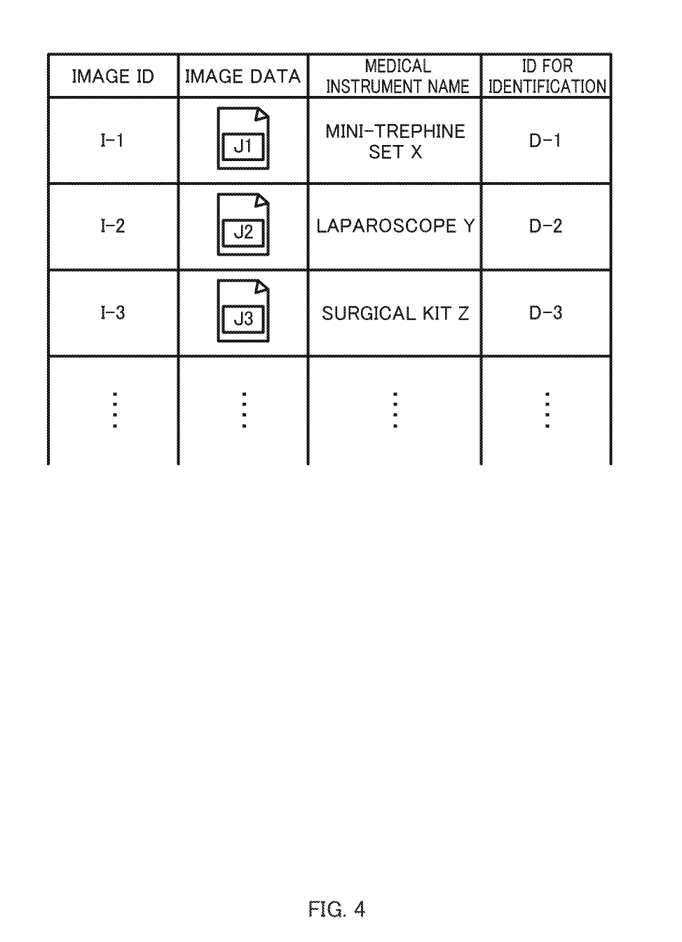

The original data are stored by, for example, taking, when a new medical instrument is bought, a picture of it using a photographing device and importing that image data into a terminal. At that time, each image data for the medical instrument is assigned with a unique identifier (image ID). FIG. 4 shows an example of the data stored in the original data storage 100. In this example, the image data are stored in the form of a table data in which image IDs as well as medical instrument names and IDs for identification of medical instruments are associated thereto. The controller 10 transmits the imported image data to the server S along with the image ID and so on.

The note data generator 200 generates a note data representing a note information that has been entered for a predetermined image data.

The note information is a piece of information representing directions in handling a medical instrument or a change in contents of a set of medical instruments. The note information may be a specific detail, such as “the tip of a curved Rochester-Pean forceps is easily broken” and “a pair of forceps for lymph nodes in the surgical set was broken and is now under repair,” entered in a text format or may be drawn as a shape, such as by circling a zone where an attention should be paid (for example, “the tip part of a curved Rochester-Pean forceps” or “a forceps for lymph nodes”).

The note information is directly entered for a predetermined image data. The entry of the note information is performed via the operator 50. The note information entered for the predetermined image data allows an operator who looks at the image to easily understand directions or change in contents of a set.

It should be noted that the entry of note information may be permitted only when a certain person such as a system administrator has been logged in a terminal or may be performed, by an operator, freely with any terminal in a hospital facility. Alternatively, the entry of note information may be performed only with a specified terminal (such as the terminal P). In such cases, even a system administrator is not allowed to enter note information using a terminal other than the terminal P1. In this way, by permitting the entry of note information under specific conditions (only a system administrator, only a specified terminal), it is possible to avoid an error such as an omission and manage note information more strictly.

The note data is generated by specifying, for example, position information (values of coordinates) of a shape or a text entered on the image data or specifying information such as a color of a shape that has been entered. The generated note data is assigned with a unique identifier (note ID). The note data generator 200 supplies the generated note data and the note ID to the note data storage 300 along with the ID for identification for the image data for which the note information has been entered. At that time, the note data generator 200 may also supply the name of a person who entered the note information (ID for an entering person), the time of entry, the place where the note information was entered, and the like. Furthermore, the note data generator 200 transmits, via the communicator 20, the generated note data to the server S along with the note ID. It should be noted that the note data is preferably a stroke data in which details of note information are drawn as a shape on a predetermined image data. The entry of note information as a shape makes it possible for operators to easily visually understand directions.

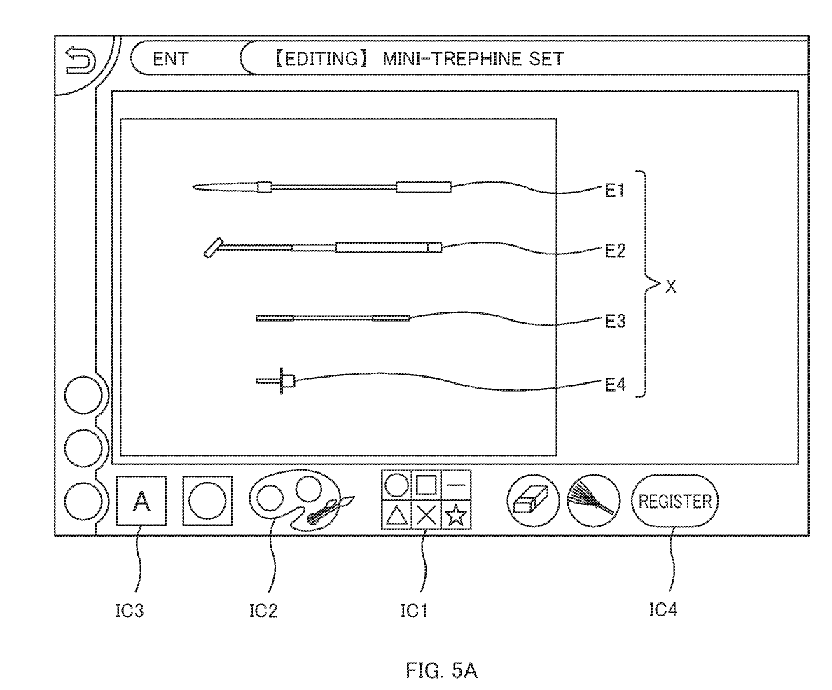

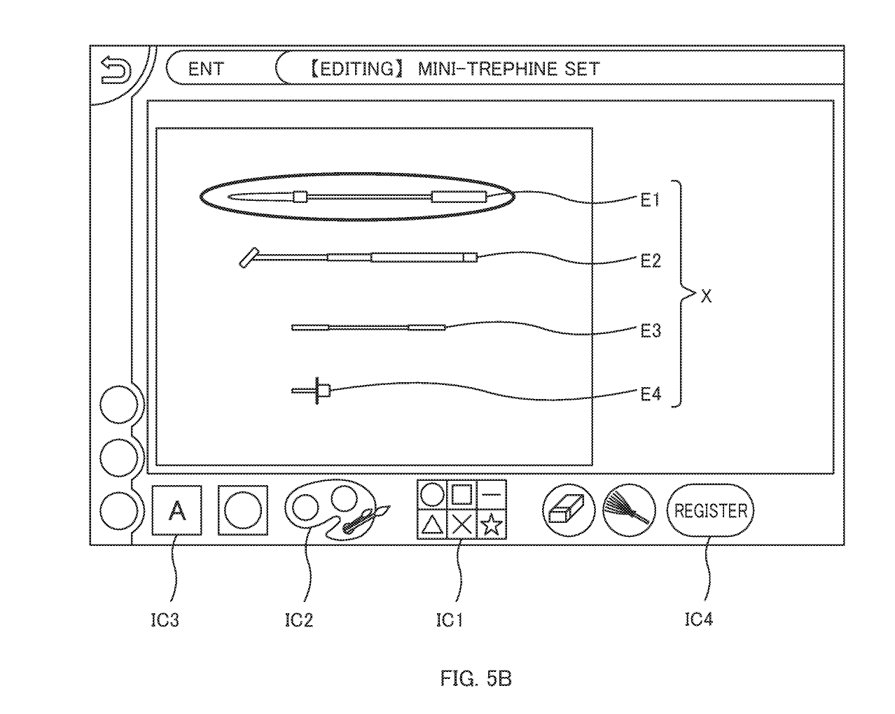

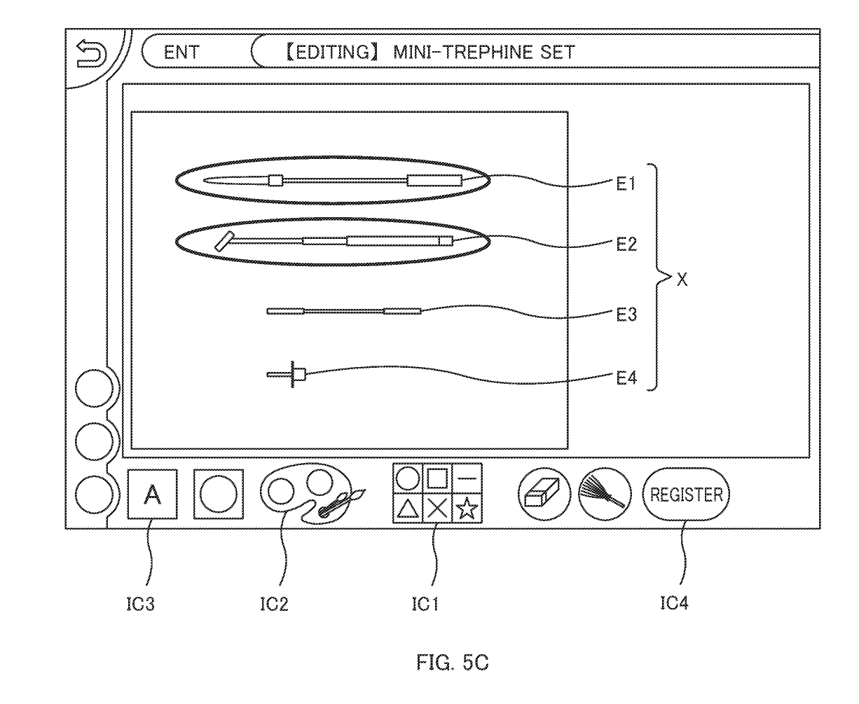

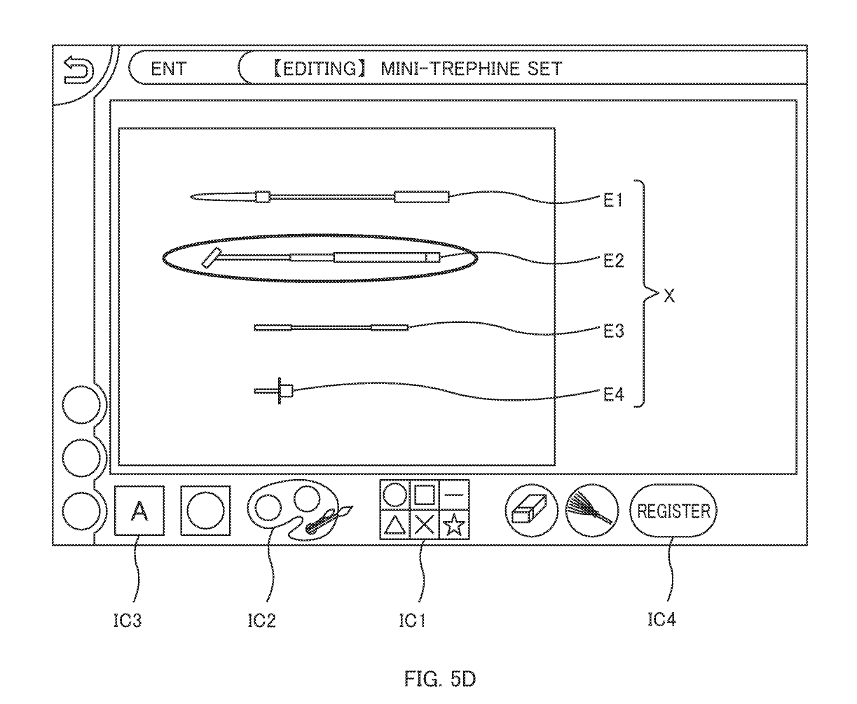

The entry of note information (generation of note data) is described using a specific example. Each of FIGS. 5A to 5D shows a display screen of the display 40. In this example, it is assumed that, for a mini-trephine set X containing four instruments E1 to E4, a note information indicating that the instrument E1 has been broken is to be entered.

A system administrator enters an ID for identification for a mini-trephine set X in an instrument selection screen displayed on the display 40 of the terminal P1. The information entered may be, for example, a medical instrument name (a part of a medical instrument name), an image ID, a department name, a sterilization method, a container, and the number of instruments. Alternatively, thumbnail images of medical instruments stored in the original data storage 100 may be displayed on the display 40 and then a system administrator may select an image from them.

The display controller 400 retrieves, based on the entered ID for identification, the original image data for the corresponding mini-trephine set X from the original data storage 100 and directs the display 40 to display an image of the mini-trephine set X (see FIG. 5A). In the display screens shown in FIGS. 5A to 5D, various kinds of icons (e.g., an “insert shape” icon IC1, a “select color” icon IC2, and an “insert text” icon IC3) for entering note information for an image and a “register” icon IC4 to confirm the entered note information. The data representing these icons are stored in the storage 30. The display controller 400 allows these icons to be displayed based on the data stored in the storage 30.

Here, it is assumed that the system administrator selects a circle in the “insert shape” icon IC1 and draws an ellipse enclosing the broken instrument E1 on a displayed image (see FIG. 5B). The note data generator 200 generates a note data M1 based on the note information (ellipse) that has been drawn. The system administrator may also enter a comment such as “out of order” as a text data in addition to the shape.

Thereafter, when the system administrator selects the “register” icon IC4, the note data generator 200 supplies the generated note data M1 to the note data storage 300 along with the note ID and so on.

Furthermore, when a new note information is entered with the note data being displayed on the predetermined image data, the note data generator 200 generates a new note data in which the new note information is merged with the note data.

For example, if an instrument E2 is also broken when the instrument E1 is out of order, then the system administrator enters, via the terminal P1, a new note information in addition to the previous note information (note information indicating that the instrument E1 is out of order) (see FIG. 5C). The note data generator 200 generates a new note data M2 by combining the added note information with the note data M1. The note data generator 200 supplies the generated new note data M2 to the note data storage 300 along with the note ID and so on.

Alternatively, if the instrument E2 is broken after the instrument E1 has been repaired, the system administrator deletes the previous note information (note information indicating that the instrument E1 is out of order) in the screen when he enters the new note information (note information indicating that the instrument E2 is out of order) (see FIG. 5D). In this case, the note data generator 200 generates a note data M3 based only on the new note information. It should be noted that, even when the previous note information (corresponding to the note data M2) is deleted in the screen and then the note data M3 is generated, the note data M2 stored in the note data storage 300 is not removed.

The note data storage 300 chronologically stores the generated note data with association to the predetermined image data. Thus, in the terminal of the present preferred embodiment, the image data for the medical instruments and the note data are stored separately.

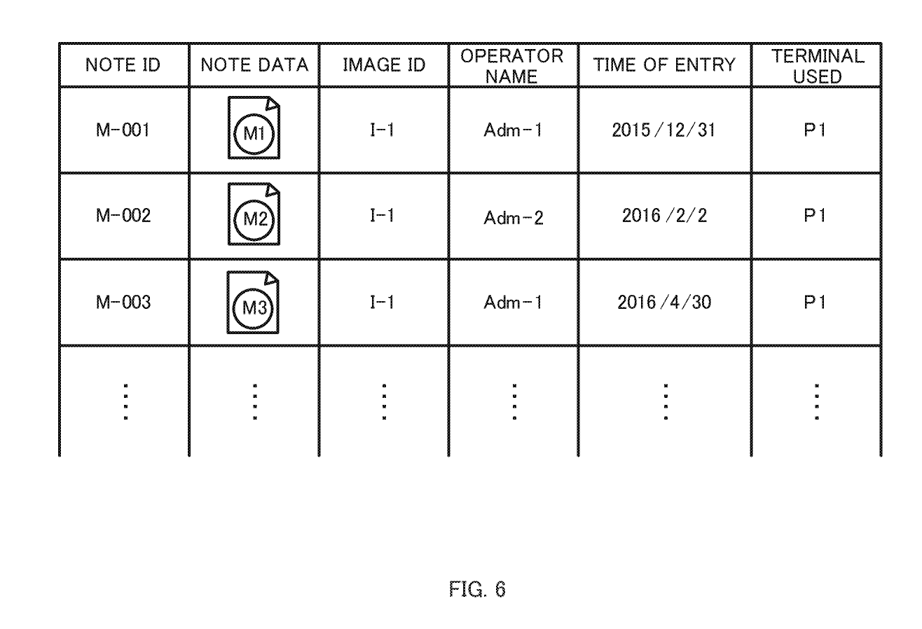

FIG. 6 shows an example of data stored in the note data storage 300. The note data are stored as a table data associated with note IDs, image IDs, IDs for entering persons, and information indicative of a terminal or terminals at which their entries have been made. For example, the note data M1 is associated with a unique note ID, an image ID representing an image data for the mini-trephine set X for which the note data M1 has been entered, an ID indicative of a system administrator who entered the note data M1, the date and time of the entry of the note data M1, and information regarding the terminal P1 at which the note data M1 was entered (see FIG. 6). Likewise, the note data M2 (M3) is associated with a unique note ID, an image ID representing an image data for the mini-trephine set X for which the note data M2 (M3) has been entered, an ID indicative of a system administrator who entered the note data M2 (M3), the date and time of the entry of the note data M2 (M3), and information regarding the terminal P1 at which the note data M2 (M3) was entered (see FIG. 6). Thus, the note data storage 300 stores the note data M1, the note data M2, and the note data M3 as separate data associated with the single mini-trephine set X.

The display controller 400 performs various controls for the display in the terminal. In particular, the display controller 400 according to the present preferred embodiment allows a note data selected from a plurality of note data that are associated with the predetermined image data to be displayed on the predetermined original image data.

Here, controls performed by the display controller 400 are described using a specific example. In the aforementioned example, the image data for the mini-trephine set X is associated with three note data M1 to M3. In this case, it is assumed that an operator intends to check note information at the terminal P1 when he performs an operation on the mini-trephine set X.

First, the operator enters an ID for identification for the mini-trephine set X in the instrument selection screen displayed on the display 40. The display controller 400 retrieves, based on the ID for identification, the original image data for the corresponding mini-trephine set X from the original data storage 100. Furthermore, the display controller 400 checks whether the note data associated with the image data for the mini-trephine set X is/are stored in the note data storage 300. In this example, the note data M1 to M3 associated with the image data for the mini-trephine set X are stored in the note data storage 300.

In this case, the display controller 400 can select, based on the date(s) and times when the note data were generated (or the order in which they were stored), a latest note data from the note data associated with the image data and allow the display 40 to display it on the image data. The latest note data is a data based on the note information reflecting particular directions at this moment or the change in contents of a set. The operator can prevent the use of a wrong procedure or know the latest content of the set by referring to such note data. Furthermore, according to the system 1 (terminal) of the present preferred embodiment, since the note data are directly displayed on the image data for the medical instrument, the operator can easily know the directions related to the handling of the medical instrument and the change in contents of the set.

The server S is a computer that accumulates and manages various kinds of information associated with medical instruments and exchanges various kinds of information with the terminals P1 to P3. Furthermore, the server S has a function of interfacing the exchange of information between or among the terminals. The server S can be installed in a hospital facility where each terminal is located or installed in a remote location such as a server provider and the like.

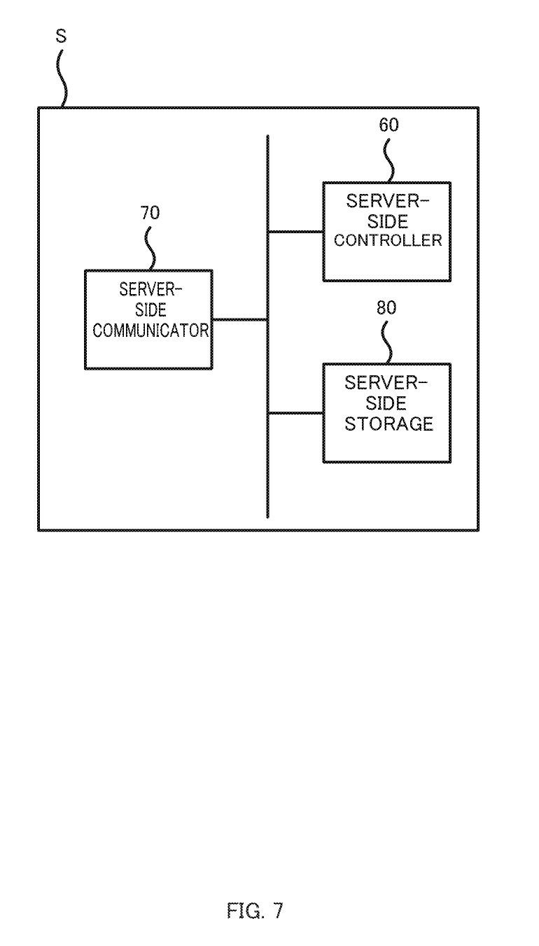

As shown in FIG. 7, the server S has a server-side controller 60, a server-side communicator 70, and a server-side storage 80.

The server-side controller 60 has a CPU and a memory (both of which are not shown). The server-side communicator 70 provides an interface for the communication between the server S and the individual terminals. The server-side storage 80 is a large-capacity storage to store various kinds of data.

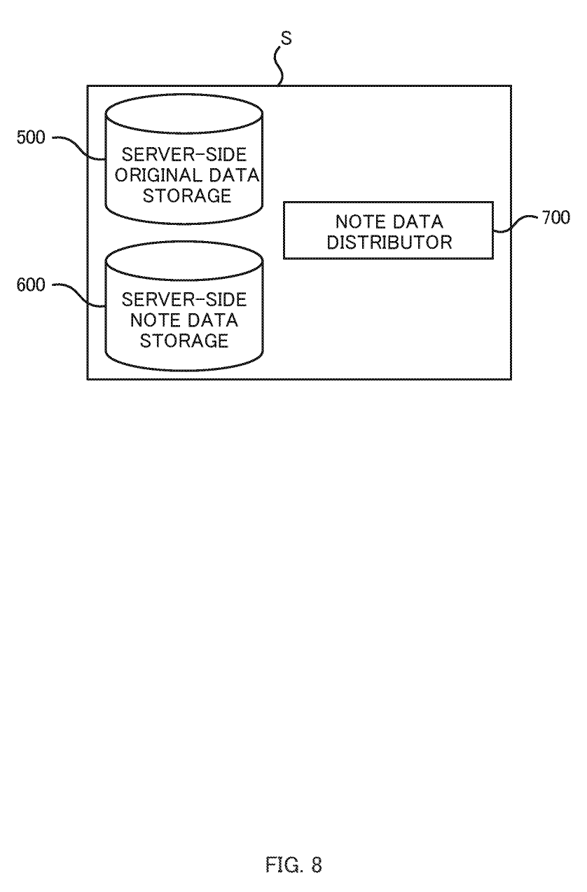

FIG. 8 is a diagram showing an exemplified software configuration in the server S. The server S includes a server-side original data storage 500, a server-side note data storage 600, and a note data distributor 700. The server-side original data storage 500 and the server-side note data storage 600 are portions of a storage region of the server-side storage 80. The note data distributor 700 is achieved when the CPU in the server-side controller 60 executes a program or programs stored in the memory.

The server-side original data storage 500 stores an image data that is identical to each original image data stored in the original data storage 100 of each terminal. When an image data for a new medical instrument is stored in a certain terminal, a copy of the image data is sent from the terminal to the server S together with its image ID and so on. The server-side original data storage 500 stores the received image data together with the image ID and so on. In other words, the image data and so on that are stored in the server-side original data storage 500 are identical to those stored in the original data storage 100 (see, for example, FIG. 4).

It should be noted that, when the server-side controller 60 receives a new image data from one terminal, it distributes the image data to the other terminals. This allows the original data storage 100 of each terminal to store the same image data for the medical instrument(s). That is, the server S and the terminals can share the same image data in real time.

The server-side note data storage 600 chronologically stores the note data transmitted from the terminal(s) with association to the predetermined image data.

For example, when the note data M1 is received from the terminal P1, the server-side note data storage 600 stores the note data M1 etc. with association to the image data for which the note data M1 has been entered. In other words, the note data etc. stored in the server-side note data storage 600 are identical to those stored in the note data storage 300 (see, for example, FIG. 6).

The note data distributor 700 distributes the note data stored in the server-side note data storage 600 to the other terminals.

When an image data is allowed to be displayed at the other terminal, the display controller 400 thereof makes an inquiry to the server S whether there is a note data that is not stored in its local note data storage 300. In response to the inquiry from the other terminal, if the note data distributor 700 determines that there is any note data not stored in the note data storage 300 of the other terminal but stored in the server-side note data storage 600, the note data distributor 700 delivers that note data to the other terminal.

For example, when the image of the mini-trephine set X is allowed to be displayed in the terminal P2, its display controller 400 makes an inquiry to the server S whether there is a new note data (a note data that is not stored in the local note data storage 300) associated with the mini-trephine set X. In response to the inquiry from the terminal P2, the note data distributor 700 determines whether there is a note data entered at the terminal P2 for the image data of the mini-trephine set X between the last time when the note data associated with the mini-trephine set X was delivered to the terminal P2 and the time when the subject inquiry was made. If any, the note data distributor 700 delivers the note data to the terminal P2. In this way, if one or more note information has been entered at other terminal(s), that information can be obtained when an image data is allowed to be displayed. As a result, the server S and the terminals can share the same note data. For example, when the note data M1 is shared, if an image of a surgical set X is allowed to be displayed at the terminal P2 or P3 other than the terminal P1 where the note information corresponding to the note data M1 has been entered, the note information corresponding to the note data M1 is displayed; therefore, the operator can check the same note information and correctly know any directions regardless of which terminal he uses.

The delivery of the note data may be made in real time. Specifically, when a note data is received from the terminal P1, the note data distributor 700 may distribute the note data to other terminals regardless of whether an inquiry is issued from one of the terminals.

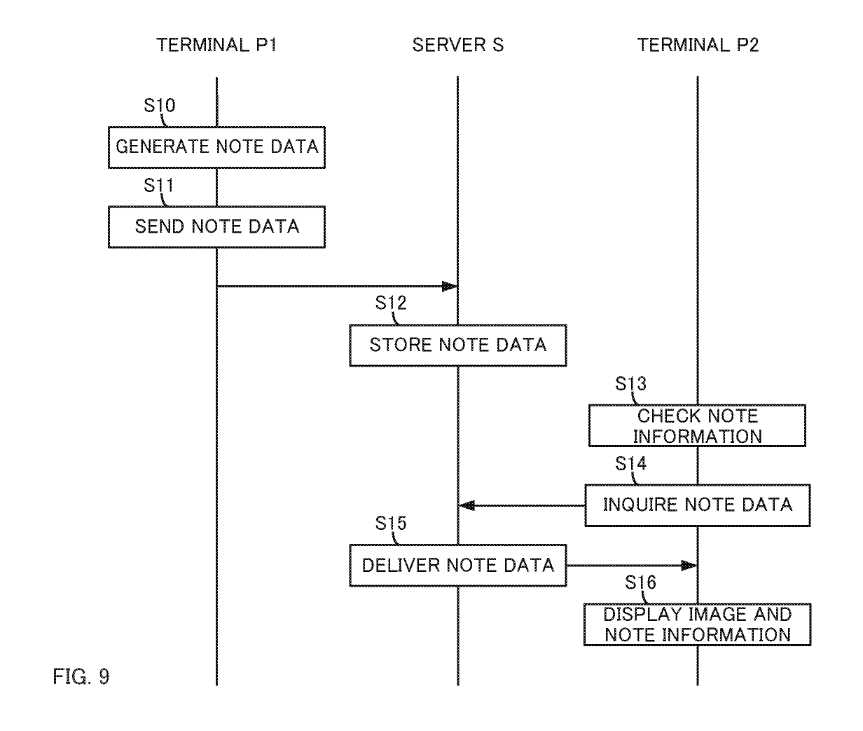

Referring to FIG. 9, management of note information in the system 1 according to this preferred embodiment is described. FIG. 9 is a flow chart illustrating a processing in the system 1. In this example, it is assumed that the server S and the terminal P2 store the image data for the mini-trephine set X beforehand. On the other hand, it is assumed that the server S and the terminal P2 do not store any note data. A description of the terminal P3 is omitted but a processing similar to the one performed in the terminal P2 can be performed in the terminal P3 as well.

First, it is assumed that, in the terminal P1, the note data M1 is generated for the image data of the mini-trephine set X (generate note data; S10).

The note data generator 200 of the terminal P1 sends, via the communicator 20, the generated note data M1 as well as a note ID and so on to the server S (send note data; S11).

The server-side note data storage 600 stores the note data M1 received from the terminal P1 with association to the image data for the mini-trephine set X (store note data; S12).

Then, it is assumed that an operator intends to check the note information of the mini-trephine set X at the terminal P2 (check note information; S13).

At this time, the operator enters the ID for identification of the mini-trephine set X in the instrument selection screen displayed on the display 40 of the terminal P2. The display controller 400 of the terminal P2 retrieves, based on the ID for identification, the original image data for the corresponding surgical set X from the original data storage 100 of the terminal P2. In addition, when the image data for the mini-trephine set X is allowed to be displayed, the display controller 400 of the terminal P2 makes an inquiry to the server S whether there is a new note data associated with the mini-trephine set X (inquire note data; S14).

In response to the inquiry from the terminal P2, the note data distributor 700 delivers, to the terminal P2, the note data M1 that has not yet been delivered thereto (deliver note data; S15).

The display controller 400 of the terminal P2 causes the display 40 to display the note data M1 on the image data for the mini-trephine set X (display image and note information; S16).

As is clear from the above, with the system 1 of the present preferred embodiment, the note information for the medical instrument(s) can be managed appropriately.

Display control by the display controller 400 can take different forms depending on needs of operators.

For example, there can be cases that an operator wants to check past note information. In such a case, the display controller 400 causes the display 40 to display a log of note data associated with the image data for the mini-trephine set X. If the operator selects the past note information via the operator 50, the display controller 400 can retrieve that note data from the note data storage 300 and display it on the corresponding image data. That is, such a device (or a system) makes it possible to check note information from a log of note information at any time point even in cases that two or more pieces of note information are added to a single image data two or more times.

Furthermore, the display controller 400 can allow only the original image data of the predetermined image data to be displayed.

For example, it is assumed that a note information corresponding to a note data associated with an image data represents a temporal information related to a failure, repair, or shortage of a medical instrument and that state has been resolved. In the example mentioned above, it is assumed that the repair of the instruments E1 and E2 has been completed and they are now ready to use. In this case, it is unnecessary to display the note information based on the note data M1 to M3. Therefore, the display controller 400 can retrieve the original image data for the mini-trephine set X from the original data storage 100 and allow the original image alone to be displayed. In this way, when a note information representing a transient state becomes unnecessary, it is easier to find the current status of the medical instrument (e.g., a state that all instruments of a set are available) by allowing only the original image data to be displayed. It should be noted that the note data M1 to M3 that are not displayed are still stored in the note data storage 300; therefore, the operator can check, if necessary, a past note information using a log record about the note data.

Furthermore, the display controller 400 can allow two or more note data to be displayed on a single image data. For example, the note data M1 and the note data M3 rather than the note data M2 may be allowed to be displayed thereon in the aforementioned example. Alternatively, when two or more pieces of note information associated with directions are entered at different times, all note data based on these pieces of note information may be allowed to be displayed on the image data.

### Second Preferred Embodiment

A second preferred embodiment of the present invention relates to computer-aided management systems for medical instruments and computer-aided management programs for medical instruments.

When a medical procedure such as a surgery or therapy is performed on a patient, it is common to use two or more medical instruments.

In performing a medical procedure, a method of managing medical instruments using a database to efficiently prepare the medical instruments to be used has been known. For example, a patent document (JP-A-2002-369787) discloses a storage that stores records of instruments used for medical procedures in a database.

To prevent medical errors (e.g., objects left in the body, in-hospital infection) involving loss of medical instrument, it is necessary to accurately record the number of the medical instruments and the instruments in each medical instrument before and after each medical procedure.

For this management, it is common to list the medical instruments on paper to be used for a certain medical procedure. Before using the medical instruments, an operator counts the number of the medical instruments close at hand by referring to the list, and checks whether the medical instruments to be used for the medical procedure are complete. In addition, the operator checks if any of the medical instruments were missing by referring to the same list after the medical procedure.

In such conventional management methods, a miscount can occur because the operator only refers to a paper list. Besides, it is hard for an operator who is not familiar with this operation to distinguish similar instruments using a paper list and therefore a miscount is more likely to occur.

Furthermore, it is hard to leave a record of a counting history (e.g., the number of what is counted, when and by whom it is counted) using the conventional management method. Accordingly, if it is found that one or more instruments are missing after a surgical operation, it is difficult to know when the instrument(s) was/were lost (e.g., the instrument(s) was/were missing before the instruments were released to an operating room or was/were lost during use in surgery).

In addition, medical instruments may be changed after a list is created in some cases (before and during surgical operations). Such an event is not recorded well because it is communicated only verbally.

In particular, since one or more persons take over the operation in time-consuming surgeries, a miscommunication on the change of medical instruments is more likely to occur. Thus, such replacement of medical instruments makes their management before and after each medical procedure harder.

Accordingly, the present preferred embodiment provides a technique with which medical instruments can be managed properly before and after medical procedures.

According to the present preferred embodiment, a computer-aided management system for medical instruments includes a display operator that allows medical instruments used for a certain medical procedure to be displayed and, when one of the medical instruments is selected, allows a count screen to be displayed, the count screen being used to perform counting of individual instruments included in the one of the medical instruments; and a count log generator that performs the counting according to a command input and creates count logs for the medical instruments, in which the display operator allows the count logs before and after the certain medical procedure to be displayed.

Furthermore, another preferred embodiment of the present invention provides a computer-aided management system for medical instruments including a display operator that allows medical instruments used for a certain medical procedure to be displayed and, when one of the medical instruments is selected, allows a count screen to be displayed, the count screen being used to perform counting of individual instruments included in the one of the medical instruments; a count log generator that performs the counting according to a command input and creates count logs for the medical instruments; and a comparator that compares the count logs before and after the certain medical procedure.

The present preferred embodiment makes it possible to accurately manage medical instruments before and after each medical procedure.

In the descriptions of the specification and the drawings that follow, at least the following features are disclosed in addition to the aforementioned preferred embodiments of the present invention.

That is, the computer-aided management system for medical instruments further including a medical instrument register that registers the medical instruments used for the certain medical procedure, in which the medical instrument register deletes some of predetermined ones of the medical instruments used for the certain medical procedure is disclosed.

Furthermore, the computer-aided management system for medical instruments in which the medical instrument register adds a new medical instrument to the predetermined ones of the medical instruments used for a certain medical procedure is disclosed.

Such a system facilitates addition and removal of the medical instrument(s) to be used. Furthermore, the system allows management of medical instruments that have been changed before and after a medical procedure.

Furthermore, the computer-aided management system for medical instruments in which the new medical instrument can be added in the meantime of the certain medical procedure, and the count log generator creates a count log for the new medical instrument is disclosed. Such a system makes it possible to manage one or more medical instruments that is/are added in the meantime of a medical procedure.

Furthermore, the computer-aided management system for medical instruments in which the display operator allows a list and/or one or more images of the individual instruments included in the medical instruments to be displayed as the count screen is disclosed. This system allows operators to appreciate the individual instruments included in each medical instrument.

Furthermore, the computer-aided management system for medical instruments in which the counting is performed by the count log generator in the case that a choice of a name of an instrument in the list or a choice of individual images, or an input of an identifier assigned to each instrument is received is disclosed. Such a system facilitates command inputs for counting by operators.

Furthermore, the computer-aided management system for medical instruments in which the count log generator adds an information of a user who has performed the counting to the count log is disclosed. Such a system makes it possible to manage the operator who has counted the number as well.

Furthermore, a non-transitory computer readable medium including a program for computer-aided management of medical instruments, the program causing a computer to display medical instruments used for a certain medical procedure; when one of the displayed medical instruments is selected, display a count screen for performing counting of individual instruments included in the one of the medical instruments; perform the counting according to a command input; create count logs for the medical instruments according to the counting; and display the count logs before and after the certain medical procedure is disclosed.

Furthermore, a non-transitory computer readable medium including a program for computer-aided management of medical instruments, the program causing a computer to display medical instruments used for a certain medical procedure; when one of the displayed medical instruments is selected, display a count screen for performing counting of individual instruments included in the one of the medical instruments; perform the counting according to a command input; create count logs for the medical instruments according to the counting; and compare the count logs before and after the certain medical procedure is disclosed.

According to these non-transitory computer readable media including programs stored thereon, it is possible to accurately manage medical instruments before and after medical procedures using a computer.

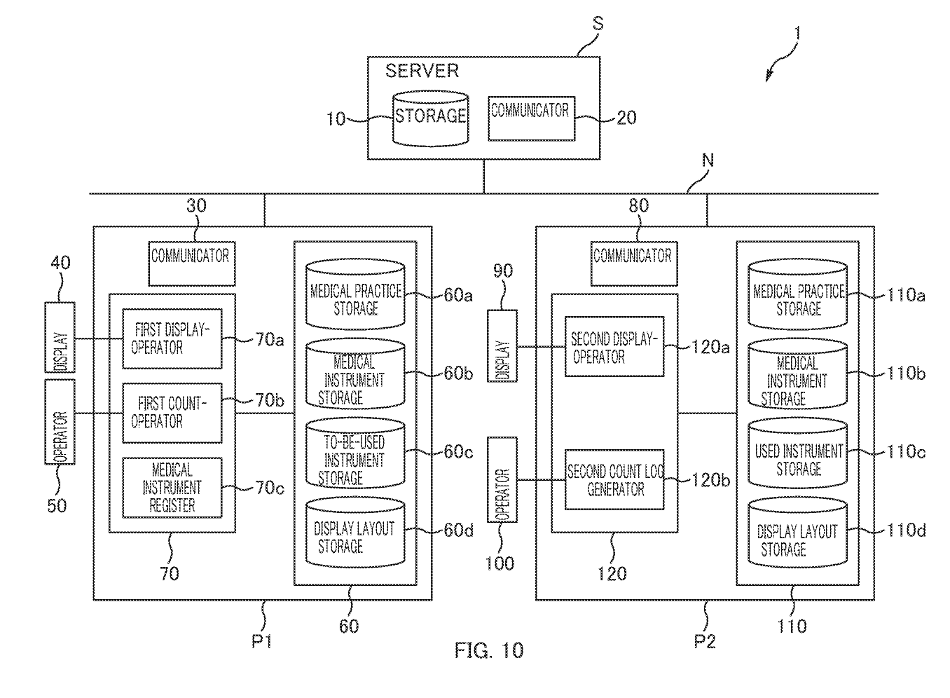

Referring to FIGS. 10 to 24, a computer-aided management system for medical instruments according to the second preferred embodiment (hereinafter, also referred to as “a system 1”) is described.

The medical procedure is, for example, a surgery, therapy, treatment, examination, etc. Medical instruments are, for example, instruments used for surgery such as pairs of surgical forceps, scalpels, and rigid endoscopes and instruments used for examinations and treatments such as pairs of biopsy forceps, ultrasonic probes, and upper and lower endoscopes. Medical instruments in this preferred embodiment include a set of two or more instruments (such as a surgical kit including pairs of forceps, scalpels, and pairs of scissors). Each medical instrument herein is assigned with a unique identifier such as a two-dimensional symbol.

As shown in FIG. 10, the system 1 of this preferred embodiment includes terminals (in this example, two terminals: a “terminal P1” and a “terminal P2”), and a server S. The terminals and the server S are connected via a network N such as an intranet, a local area network (LAN), or the Internet so that they can communicate with each other via cables or wirelessly. Each terminal is a desktop personal computer or a mobile terminal (such as a laptop PC and a tablet computer).

The server S is a computer that accumulates and manages various kinds of information as well as exchanges information with the terminals. The server S has a function of interfacing the exchange of information between or among the terminals. The server S can be installed in a hospital facility where each terminal is located or installed in a remote location such as a server provider and the like.

The server S includes a storage 10 and a communicator 20. The storage 10 (server-side storage) is a large-capacity storage that stores information regarding electronic medical records and information regarding medical instruments. The storage 10 of the present preferred embodiment stores count logs (described later) transmitted from the terminals. The communicator 20 exchange data with the terminals via the network N.

The terminals P1 and P2 are devices capable of displaying medical instruments used for medical procedures and sending and receiving information to and from the server S. The terminals can be located at different areas in a hospital facility. For example, the terminal P1 is located in an area where medical instruments before use are stored (an area where medical instruments are to be released) and the terminal P2 is located in an area where used medical instruments are collected. In this preferred embodiment, the term “before the use (after the use) of a medical instrument” is equivalent to “before a medical procedure (after a medical procedure) (for which the medical instrument is used).”

The terminal P1 of the present preferred embodiment functions as a device for performing, before a certain medical procedure, counting of the instruments included in a medical instrument used for the medical procedure and creating a count log. The terminal P1 includes a communicator 30, a storage 60, and a controller 70, and is connected to a display 40 and an operator 50.

The communicator 30 exchange data with the server S and the terminal P2 via the network N. The display 40 displays, for example, images of medical instruments and a list of individual instruments. The display 40 and the terminal P1 may be one unit. The operator 50 is an input interface such as a mouse. Alternatively, the display 40 of a touch-panel screen type may double as the operator 50. The operator 50 and the terminal P1 may be one unit. An operator enters commands to the terminal P1 via the operator 50.

The storage 60 is a large-capacity storage that stores various kinds of data. As shown in FIG. 10, in this preferred embodiment, a medical procedure storage 60a, a medical instrument storage 60b, a to-be-used instrument storage 60c, and a display layout storage 60d are portions of a storage region of the storage 60.

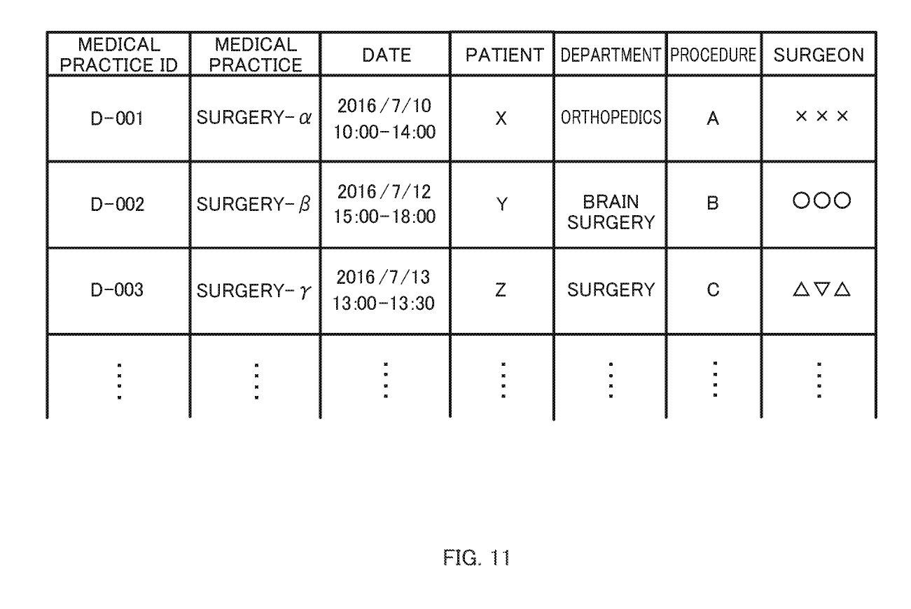

The medical procedure storage 60a stores information regarding a medical procedure planned to be performed on a certain patient. FIG. 11 is an example of data stored in the medical procedure storage 60a. As shown in FIG. 11, for each medical procedure to be performed, identification information (medical procedure ID), the kind of the medical procedure, the date on which the medical procedure is to be performed, the patient name, the department name, the procedure, and the surgeon are stored with association to each other. The “procedure” is a type of the medical procedure (e.g., extirpation of . . . , resection of . . . , and treatment of . . . ).

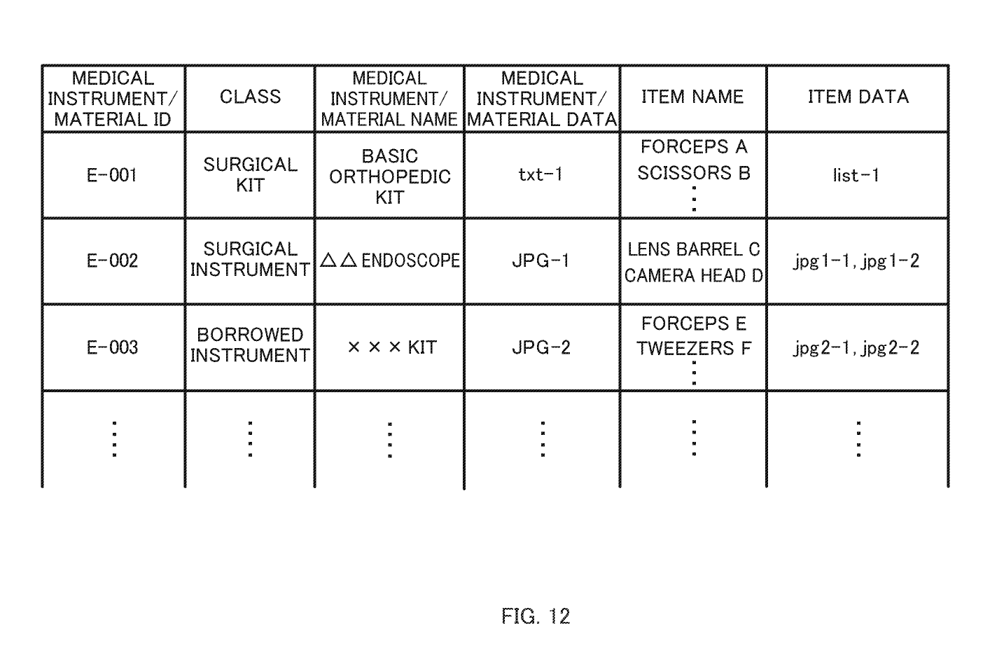

The medical instrument storage 60b stores data on the medical instruments. FIG. 12 shows an example of data stored in the medical instrument storage 60b. As shown in FIG. 12, for each medical instrument, identification information (medical instrument ID), the type, the name of the medical instrument, medical instrument data, names of individual instruments (item name), individual instrument data (item data) are stored with association to each other.

The “class” is for classifications of medical instruments. In this preferred embodiment, medical instruments indicated as the “surgical kit” are, for example, a set of small steel articles used for surgery and treatment, such as forceps and scissors. In such medical instruments, individual pairs of forceps and scissors are the instruments (individual items) herein. Medical instruments indicated as the “surgical instrument” are special tools such as rigid endoscopes for surgery. In such medical instruments, components and parts of each rigid endoscope (e.g., a lens barrel and a camera head) are the instruments (individual items). Furthermore, medical instruments indicated as the “borrowed instrument” are those on loan from a manufacturer for a surgery or personal belongings of doctors. The medical instruments and individual instruments include disposable products (e.g., gauze, catheters, and injection needles) and implants (e.g., artificial joints and stents). In addition, the classes of the “surgical kit,” “surgical instrument,” and “borrowed instrument” are mere examples and users do not have to follow this classification. For example, the “surgical kit” and the “surgical instrument” may be combined as a single class.

The “medical instrument data” is for data for displaying medical instruments on the display 40. Each medical instrument data is, for example, a text data (indicated by “txt” in FIG. 12) representing the name of a medical instrument or an image data (indicated by “JPG” in FIG. 12) obtained by photographing a medical instrument beforehand using an image-pickup device. The “individual instrument data” is a data for displaying each instrument on the display 40. The individual instrument data is, for example, a data of a list containing individual instruments included in one medical instrument (indicated by “list” in FIG. 12) or an image data obtained by photographing each instrument beforehand using an image-pickup device (indicated by “jpg” in FIG. 12).

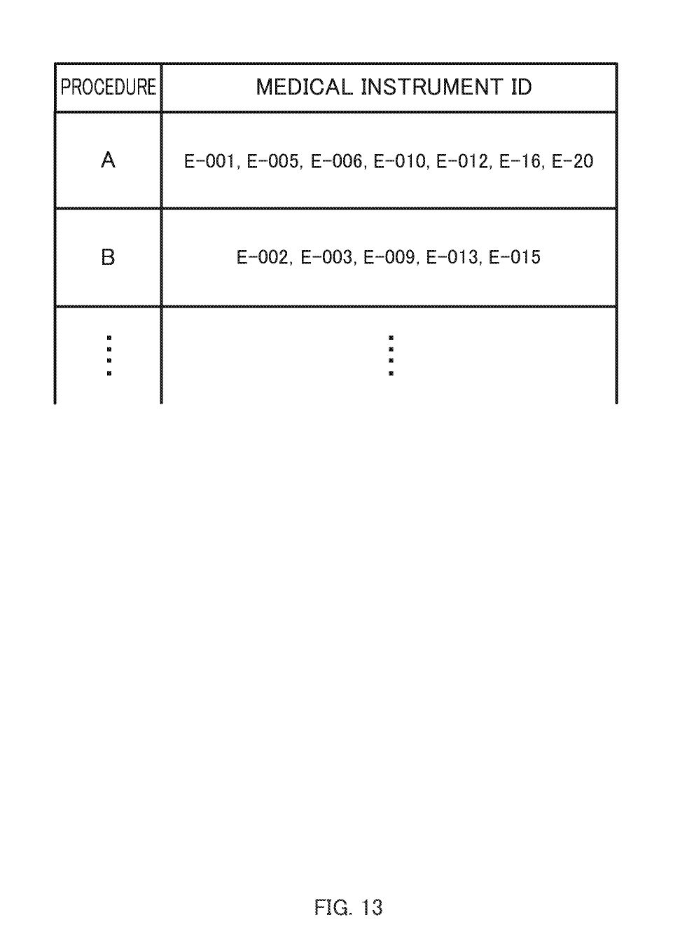

In the to-be-used instrument storage 60c, one or more medical instruments that are used for a certain medical procedure are stored. The medical instrument(s) that are used for a certain medical procedure is/are determined beforehand. FIG. 13 is an example of a data stored in the to-be-used instrument storage 60c. As shown in FIG. 13, two or more medical instruments (medical instrument IDs) are related to each procedure. These medical instruments indicate medical instruments that have a strong likelihood of being usually used for a certain procedure. What are related to the medical instruments are not limited to procedures. For example, two or more medical instruments may be related to each medical procedure.

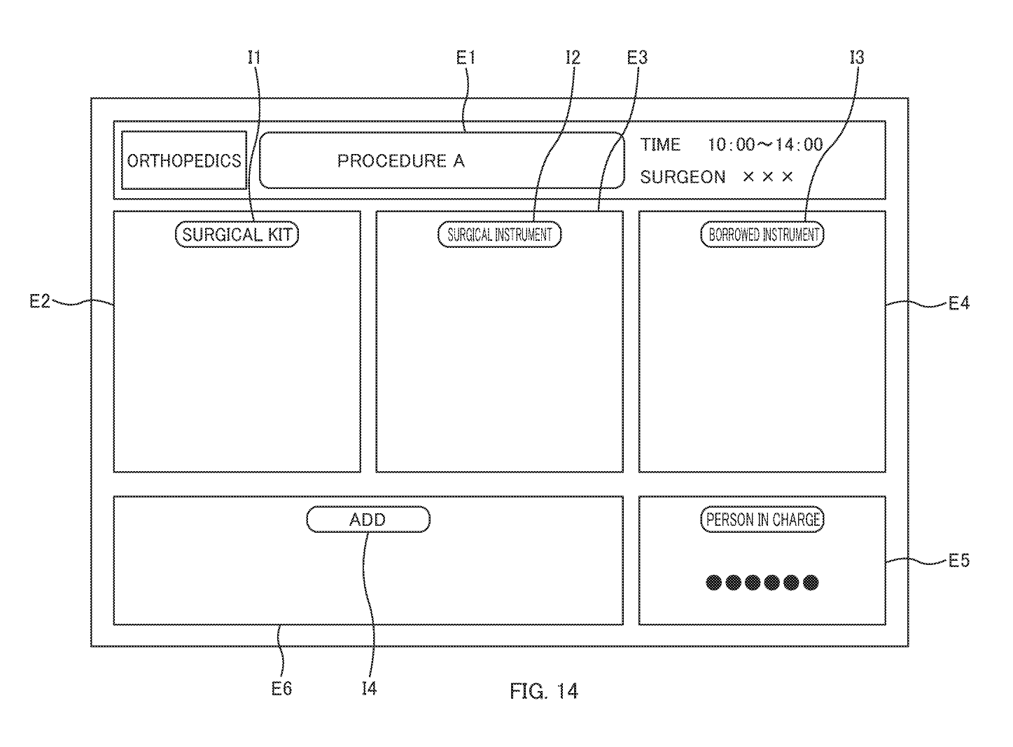

The display layout storage 60d has layout data for various display screens (see, for example, FIG. 14). A first display operator 70a (described later) provides display screens on the display 40 based on the layout data.

It should be noted that some or all of the data stored in the storage 60 may be stored in, for example, the storage 10 of the server S.

The controller 70 has a CPU and a memory (which are not shown). The CPU achieves different kinds of control functions by executing an operating program stored in the memory. The memory is a storage that stores a program or programs executed by the CPU or temporarily stores various pieces of information upon execution of the program(s). The controller 70 executes, based on signals (command inputs) from the operator 50, corresponding processing operations.

The controller 70 of this preferred embodiment defines and functions as the first display operator 70a, a first count log generator 70b, and a medical instrument register 70c. It should be noted that the medical instrument register 70c is not an essential component in various preferred embodiments of the present invention.

The first display operator 70a allows medical instruments used for a certain medical procedure to be displayed and, when one of the medical instruments is selected, allows a first count screen to be displayed which is used for performing the counting of individual instruments included in the one of the medical instruments.

The medical instruments are displayed on the basis of the medical instrument data stored in the medical instrument storage 60b. In addition, a list and/or one or more images of the individual instruments included in the medical instruments is/are displayed on the first count screen. The first display operator 70a allows the list and/or the image(s) to be displayed based on the individual instrument data stored in the medical instrument storage 60b. The first display operator 70a is an example of the “display operator” and the first count screen is an example of the “count screen.”

The first count log generator 70b increments according to a command input and creates one or more count logs for each medical instrument.

For example, an operator selects an instrument/material that is kept close at hand from the list of the individual instruments/materials displayed on the first count screen (an example of the command input). The first count log generator 70b increments according to this choice. If each instrument/material has a unique identifier, the operator uses a reader (not shown) connected to the terminal P1 to read the identifiers (an example of the command input). The first count log generator 70b is incremented for the instrument corresponding to the identifier based on the identifier (input identifier) that have been read. The first count log generator 70b is an example of the “count log generator.”

After the number of all instruments included in a certain medical instrument has been checked, the first count log generator 70b creates log information (a count log) according to the count result. Such a count log (a count log before a certain medical procedure) is stored with association to a medical procedure ID and time information indicative of the time when the counting was performed. For example, the first count log generator 70b sends the count log to the server S via the communicator 30. The server S allows the count log received through the communicator 20 to be stored in the storage 10.

It should be noted that the first count log generator 70b may allow the count log etc. to be stored in the storage 60 of the terminal P1. Alternatively, the first count log generator 70b may send the count log, etc., indirectly through the server S or directly over the network N to the terminal P2 and allow the count log to be stored in a storage 110 (described later) of the terminal P2.

Furthermore, the first count log generator 70b of the present preferred embodiment can add user information to specify the operator who has performed the counting (e.g., the name of the operator, an operator ID) to the count log. The operator who has counted the number can be specified by, for example, checking a history of logging in to the terminal.

Now, referring to FIGS. 14 to 17D, for a medical instrument used for a surgery α on a patient X, specific examples of display operations and count operations for performing the counting of individual instruments included in the medical instrument on the terminal P1 are described. FIGS. 14 to 17D show screens displayed on the display 40. It is assumed that the operator enters various inputs (selections) via the operator 50.

Before releasing medical instruments to an operating room where the surgery α is performed, the operator checks whether the medical instruments necessary for the surgery α are complete.

To do this, the operator logs in to the system 1 through the terminal P1 and enters the medical procedure ID for the surgery α. The first display operator 70a retrieves the information (see FIG. 11) about the surgery α from the medical procedure storage 60a and causes the display 40 to display it (see FIG. 14). In this example, among the pieces of information regarding the surgery α, the department (orthopedics), procedure, date (only the time in FIG. 14), and surgeon are displayed.

In addition, in this example, the name of the operator who has logged in is displayed in an area E5. It should be noted that information regarding the surgery α on the patient X may be read from electronic medical record information stored in the server S or the storage 60 and displayed.

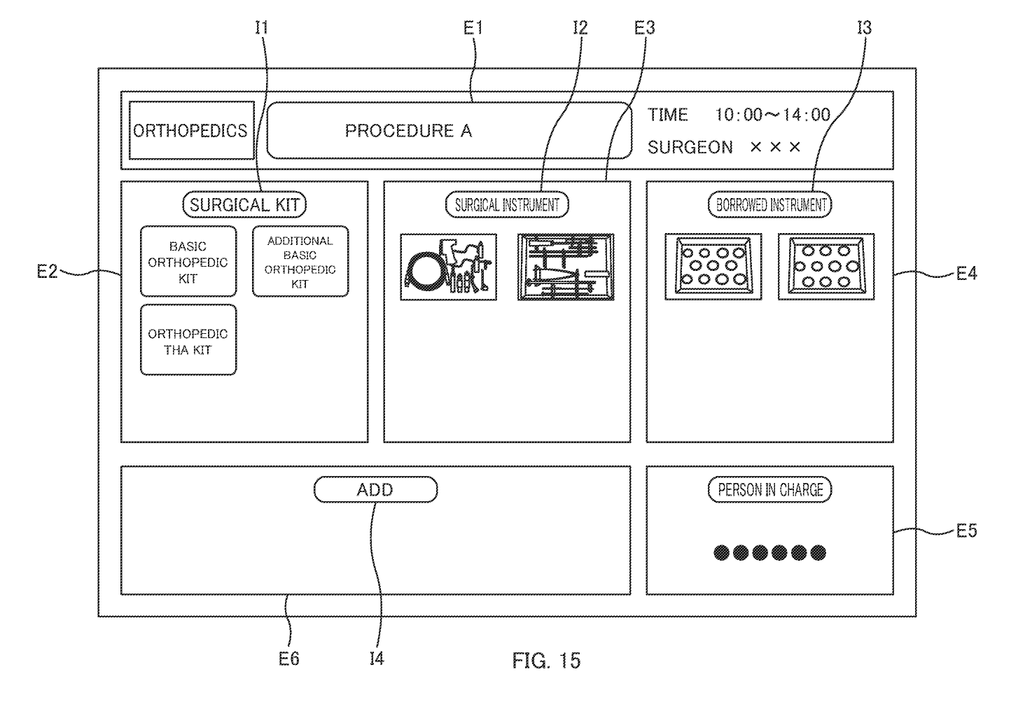

Next, the operator selects an area E1 where a procedure is displayed. In response to the selection, the first display operator 70a retrieves an ID of each medical instrument used for a procedure A from the to-be-used instrument storage 60c. Next, the first display operator 70a retrieves data related to the medical instrument corresponding to that ID from the medical instrument register 60b. The first display operator 70a allows a text or an image for the medical instrument for each class to be displayed based on the class included in that data and the medical instrument data (see FIG. 15). In this example, three texts for medical instruments are displayed in an area E2 where the surgical kit(s) is/are displayed, two images for medical instruments are displayed in an area E3 where the surgical instrument(s) is/are displayed, and two images for medical instruments are displayed in an area E4 where the borrowed instrument(s) is/are displayed.

Then, the operator counts the number of individual instruments included in each displayed medical instrument.

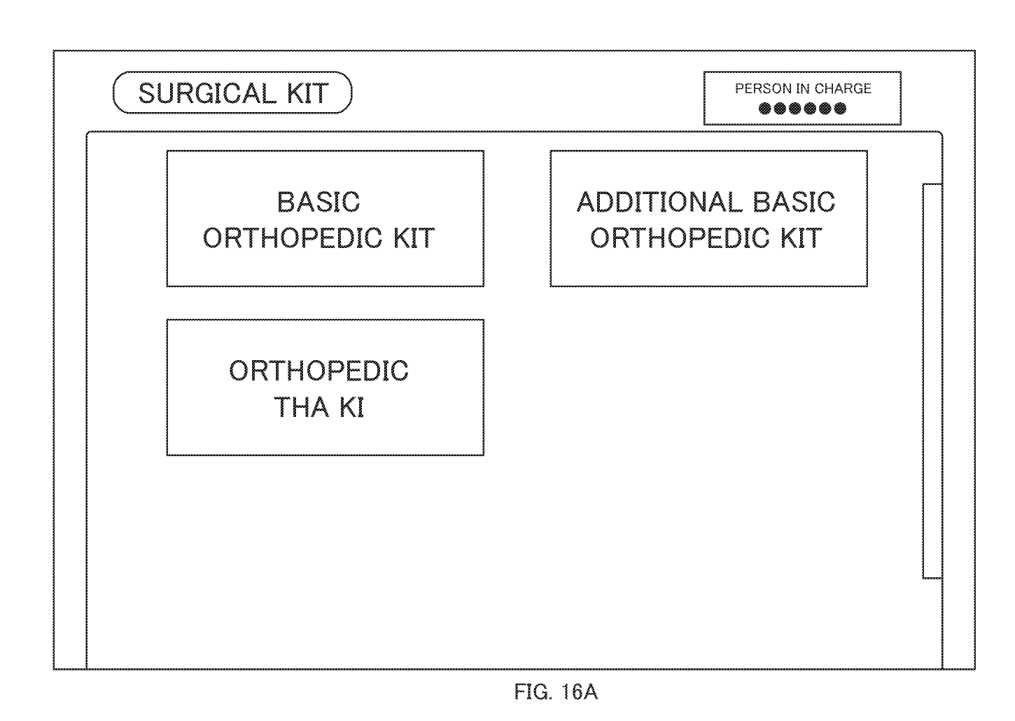

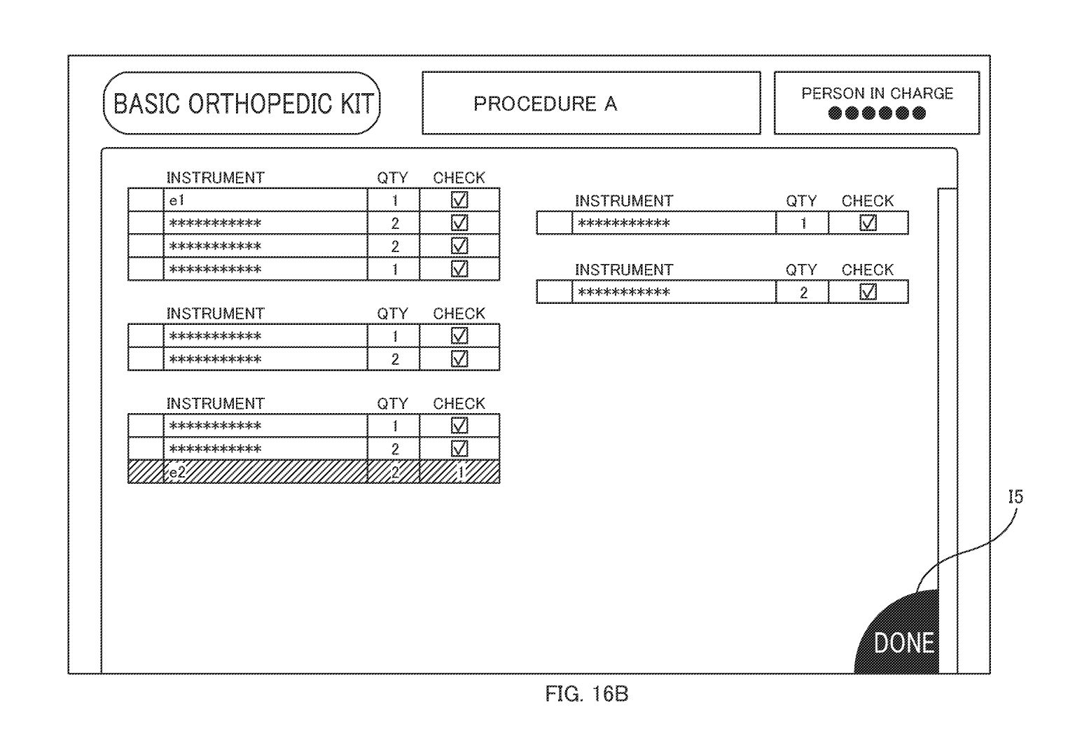

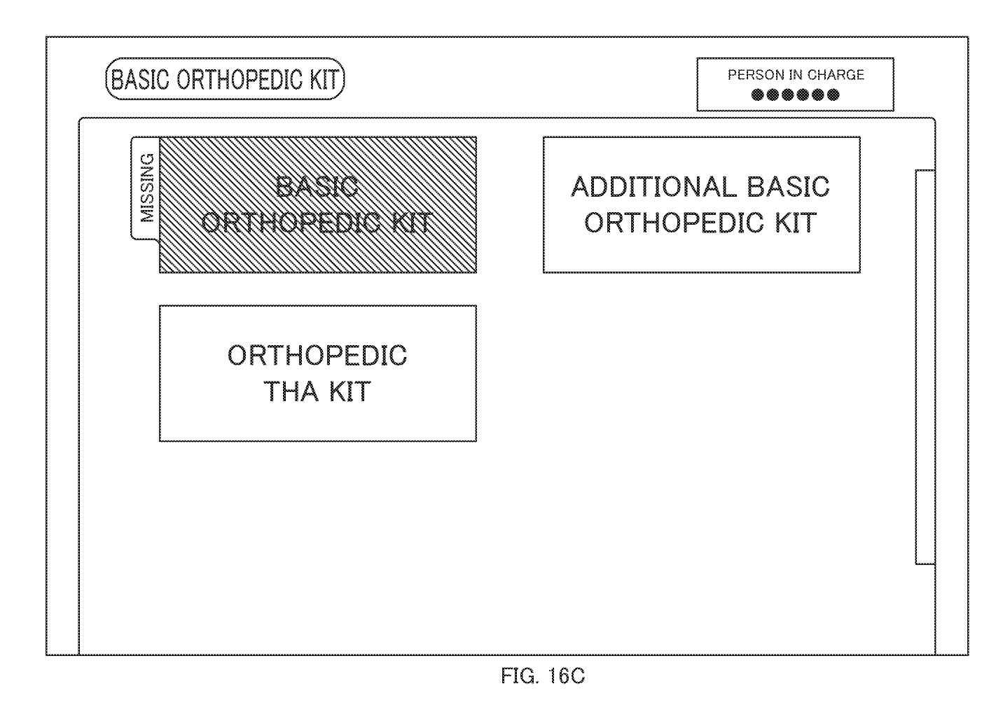

First, an example of performing the counting using a list of the instruments is described. The operator selects the area E2 for the surgical kits. In response to this selection, the first display operator 70a allows a screen for selecting a medical instrument included in a surgical kit to be displayed (see FIG. 16A). The operator selects the text of the medical instrument for which the number of the individual instruments included in it is to be counted. In this example, it is assumed that the “basic orthopedic kit” is selected among the text strings displayed in the area E2.

The first display operator 70a allows a count screen to be displayed for use in performing the counting of the individual instruments in the basic orthopedic kit (see FIG. 16B). Specifically, the first display operator 70a allows a list of instruments to be displayed based on item data related to the basic orthopedic kit (data obtained by listing the names of the instruments included in the basic orthopedic kit). In the count screen, the names of the instruments, the number of the instruments (the number of the content items) and checkboxes are displayed.

The operator examines the basic orthopedic kit before serving and determines whether the instruments are complete. For example, when the number of the instruments E1 that are kept close at hand matches the number of content items for the instruments E1 displayed in the count screen, the operator selects on the checkbox. In response to the selection, the first count log generator 70b is incremented for the instruments E1 and allows a check mark to be displayed in the checkbox.

On the other hand, if the number of the instruments E2 that are kept close at hand does not match the number of the content items for the instruments E2, the operator selects the area for the number of content items for the instruments E2. For example, if one of the instruments E2 is missing, the operator selects on the area for the number of content items for the instruments E2 once. In this case, in response to this selection, the first count log generator 70b is incremented for the instruments E2 and allows the number of missing instruments “1” to be displayed in the checkbox. FIG. 16B is an example of the “first count screen.”

After the counting of all instruments has been completed, the operator selects a “done” icon I5 (see FIG. 16B). The first count log generator 70b then creates a count log for the basic orthopedic kit according to the check result (count result).

Furthermore, the first display operator 70a allows the medical instruments of which counting has been completed to be displayed in such a manner that users can distinguish them from uncounted medical instruments (such as by using different colors for them) (see FIG. 16C). In addition, if one or more instruments are missing, the first display operator 70a may allow a textbox for a medical instrument to be displayed together with a tab indicating that one or more instruments are missing (see FIG. 16C).

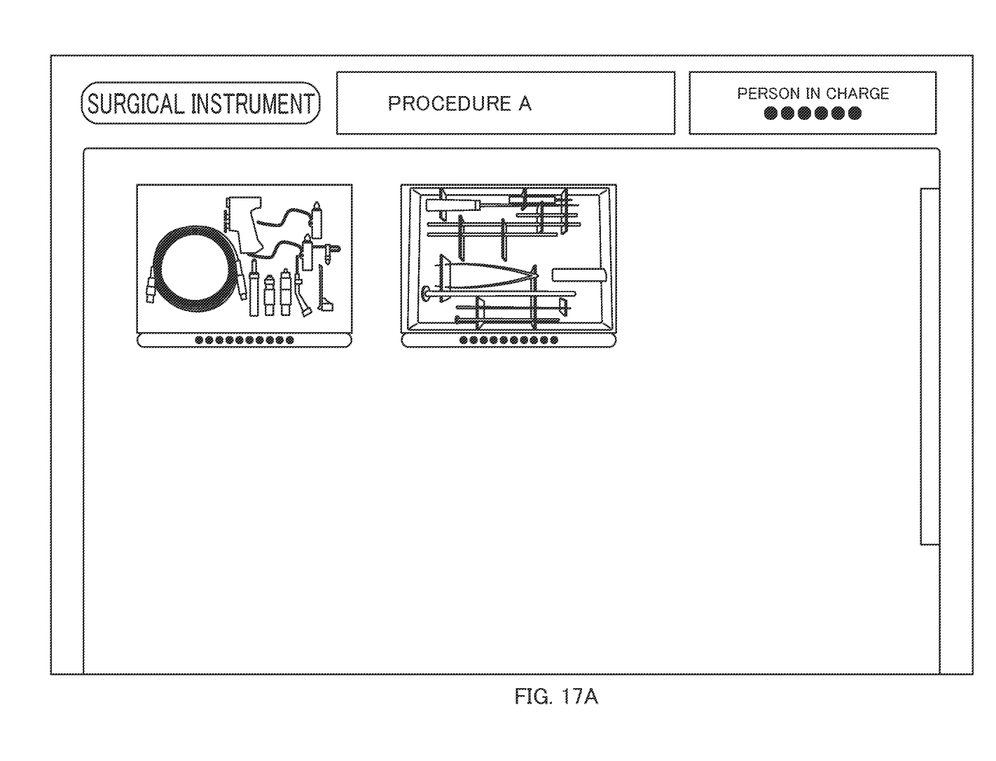

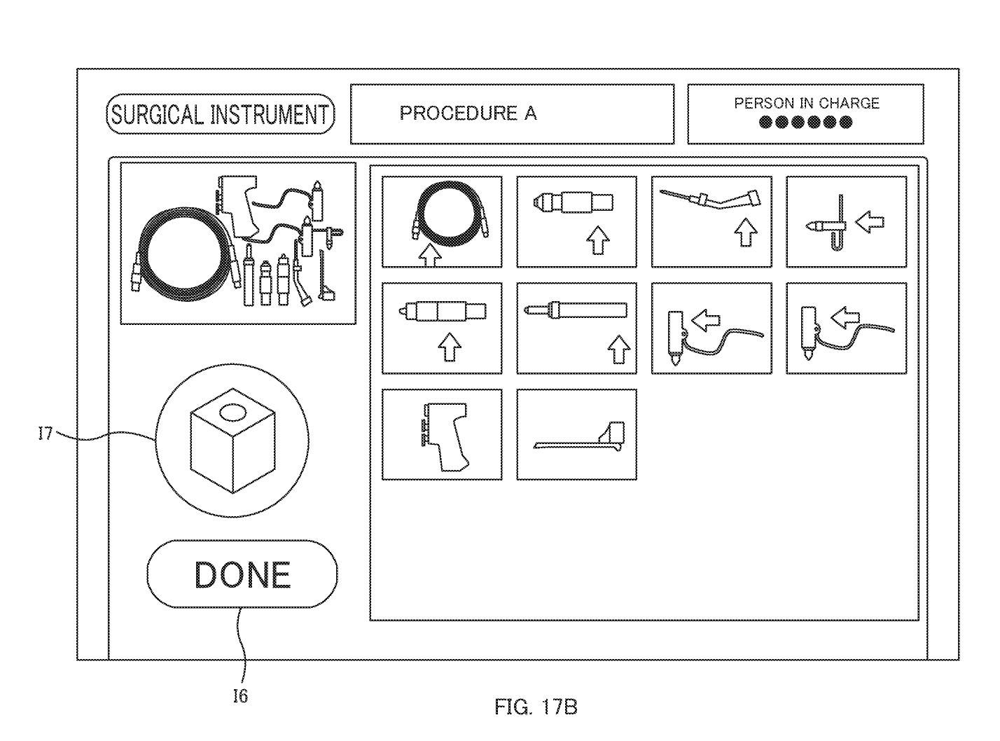

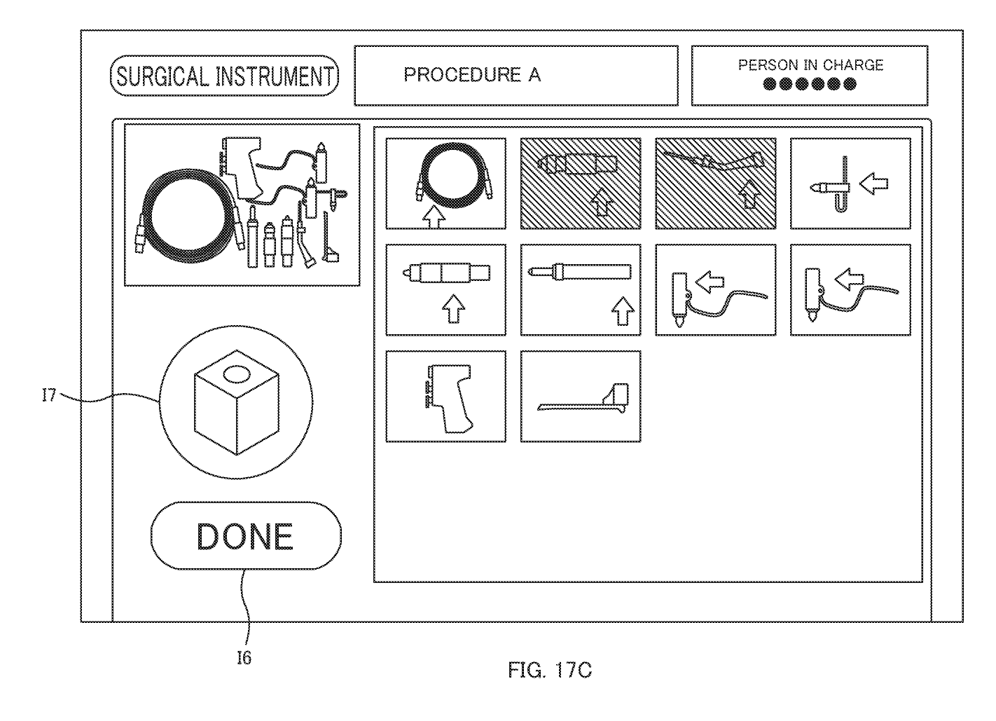

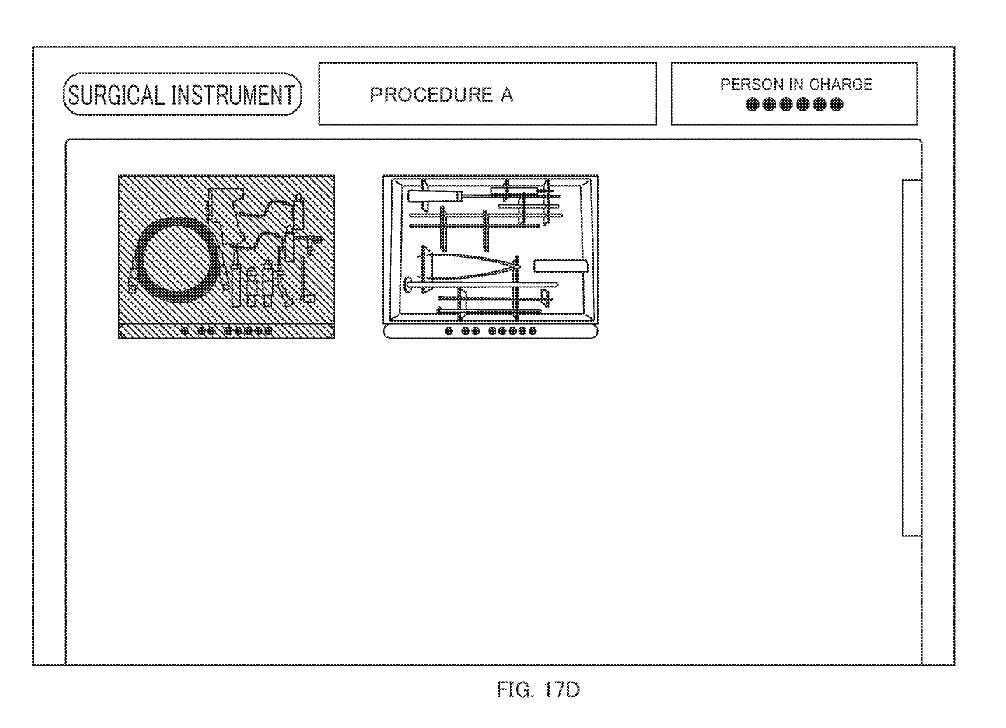

Next, an example of performing the counting using images of individual instruments is described. When the operator selects the area E3 for the surgical instruments, the first display operator 70a allows, in response to the selection, a screen for selecting a medical instrument included in the surgical instrument to be displayed (see FIG. 17A). The operator selects an image of a medical instrument for which the number of the individual instruments included in it is to be counted.

The first display operator 70a allows a count screen to be displayed for use in performing the counting of the individual instruments included in the selected medical instrument (see FIG. 17B). Specifically, the first display operator 70a allows images of instruments to be displayed based on item data related to the selected medical instrument (image data of the instruments included in the medical instrument).

The operator examines the medical instrument before serving and determines whether the instruments are complete. For example, if an image identical to the instrument kept close at hand is displayed on the count screen, the operator selects that image. In response to the selection, the first count log generator 70b is incremented for the instruments and the image gets the selected appearance (see FIG. 17C). FIGS. 17B and 17C are examples of the “first count screen.”

After the counting of all instruments has been completed, the operator selects a “done” icon I6. The first count log generator 70b then creates a count log for the medical instrument according to the check result (count result).

Furthermore, the first display operator 70a allows the medical instruments of which counting has been completed to be displayed in such a manner that users can distinguish them from uncounted medical instruments (see FIG. 17D).

Furthermore, some instruments made of metal are tagged with a unique identifier such as a two-dimensional symbol. In such cases, the counting may be performed by reading the identifier.

The operator selects a “reader” icon I7 (see, for example, FIG. 17B). In response to the selection of the “reader” icon I7, a reader (not shown) connected to the terminal P1 is activated. The operator uses the reader to read identifiers assigned to the instruments. In response to the inputs of the identifiers from the reader, the first count log generator 70b is incremented for the instruments.

It should be noted that it can be hard for operators to find identifiers assigned to instruments because they are small. Thus, arrows that indicate a place where an identifier is located may be provided in the displayed images of the individual instruments (see, for example, FIG. 17B). The images with the arrows are stored previously in the medical instrument storage 60b as individual instrument data. Using such images eliminates the necessity of finding an identifier, so that the operator can check the instruments in an efficient manner.

After the counting of all medical instruments has been completed, the first count log generator 70b sends a created count log to, for example, the server S. The server S allows the count log (a count log before a medical procedure) to be stored in the storage 10 with association to, for example, the medical procedure ID. On the other hand, after the checking of all medical instruments has been completed, the operator releases the medical instruments.

It should be noted that, unlike the above example, each medical procedure ID may previously be related to the medical instrument(s) (medical instrument ID(s)) to be used. In such cases, when a medical procedure ID for the surgery α is entered on the display screen, the first display operator 70a retrieves data related to the medical instrument that is related to the surgery α from the medical instrument register 60b. The first display operator 70a allows a text or an image for the medical instrument for each class to be displayed based on the class included in that data and the medical instrument data. This saves the operator a step of selecting a procedure and thus enables easier display of the medical instruments. In this case, the to-be-used instrument storage 60c is unnecessary.

In the case where a medical instrument includes an implant or a disposable product, the first display operator 70a can allow it to be displayed in a list or an image in such a manner that users can distinguish them from other instrument (such as by using a different color for letters in the list or a display pane or using a different color for the border of the image).

Although the above example has been described for cases in which one medical instrument is displayed in either a text or image format, the present invention is not limited to this. It is also possible to display both texts and images for one medical instrument. In this case, each medical instrument data stored in the medical instrument storage 60b is related to both image data and text data.

The medical instrument register 70c registers one or more medical instruments used for a certain medical procedure.

As described above, medical instruments used for each medical procedure are predetermined depending on, for example, its procedure. Accordingly, it is usually enough to perform checking (count operation) for the medical instruments displayed on the display screen before releasing them.

For some patients and conditions, however, it is likely that one or more of the medical instruments are not required or an additional medical instrument is be added. In such cases, it becomes necessary to change (add or remove) the medical instruments that are registered beforehand in the to-be-used instrument register 60c. The medical instrument register 70c performs change operations for such medical instruments.

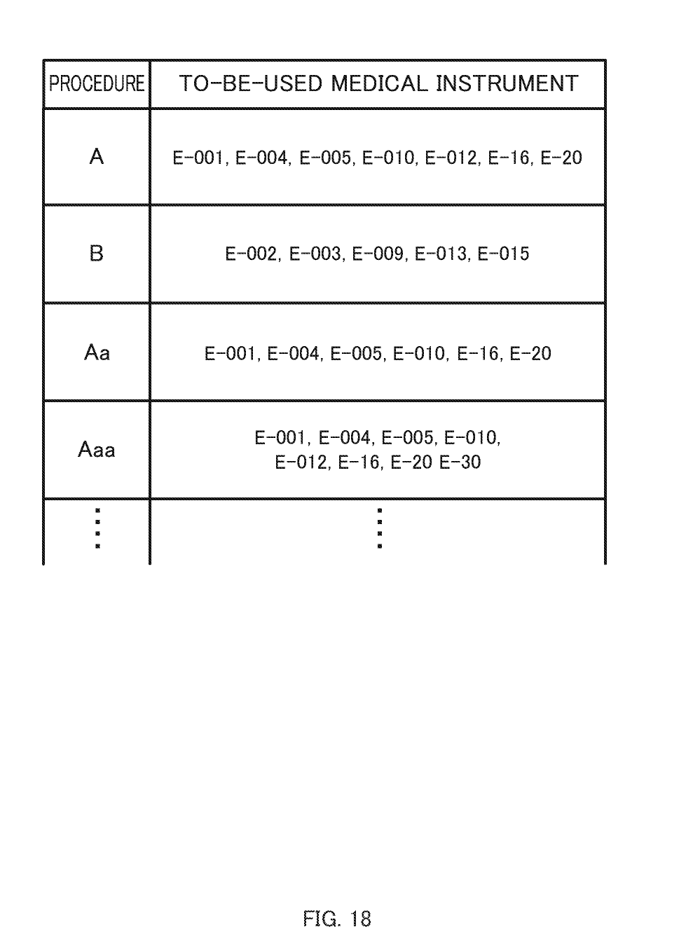

Specifically, the medical instrument register 70c deletes a part of a predetermined one or ones of the medical instruments to be used for a certain medical procedure. Alternatively, the medical instrument register 70c adds a new medical instrument to the predetermined one or ones of the medical instruments used for a certain medical procedure. The medical instrument register 70c allows information of the changed medical instrument(s) to be stored in the to-be-used instrument storage 60c. For example, when a part (medical instrument ID: E-012) of the medical instrument is deleted for the procedure A applied to the surgery α on the patient X, information of the new medical instrument is stored in the to-be-used instrument storage 60c as a procedure Aa (see FIG. 18). Likewise, when a certain medical instrument (medical instrument ID: E-030) is added for the procedure A applied to the surgery α on the patient X, information of the new medical instrument is stored in the to-be-used instrument storage 60c as a procedure Aaa (see FIG. 18).

Furthermore, the medical instrument register 70c updates the information in the medical procedure storage 60a. For example, in the above example, the medical instrument register 70c updates the procedure applied to the surgery α on the patient X from the procedure A to the procedure Aa (or the procedure Aaa). The medical instrument register 70c sends the updated information to, for example, the server S. The server S allows the updated information to be stored in the storage 10.

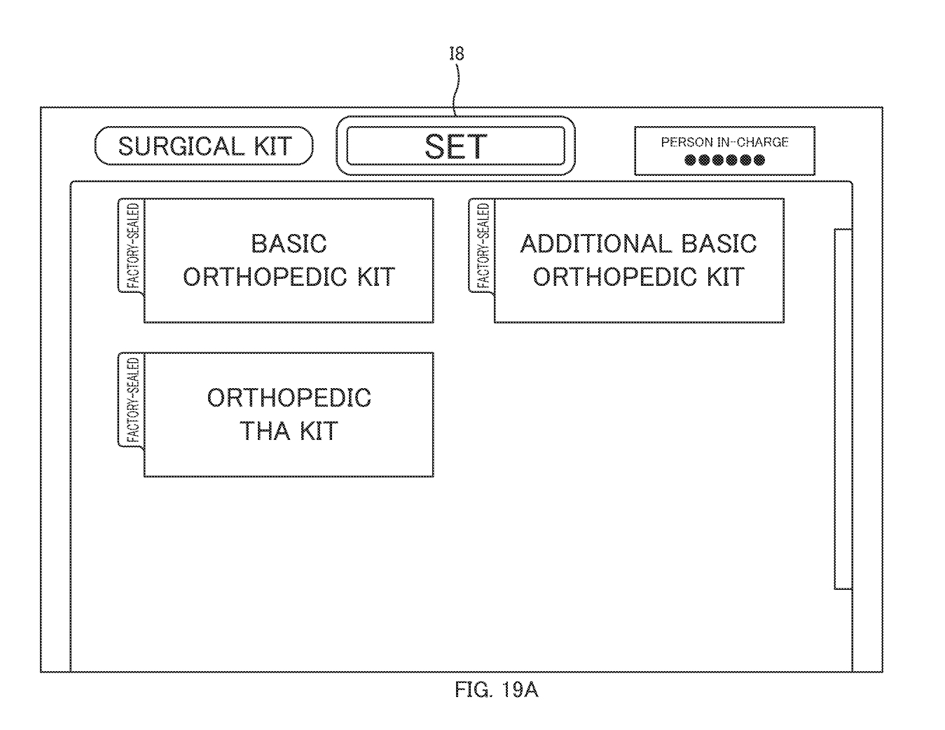

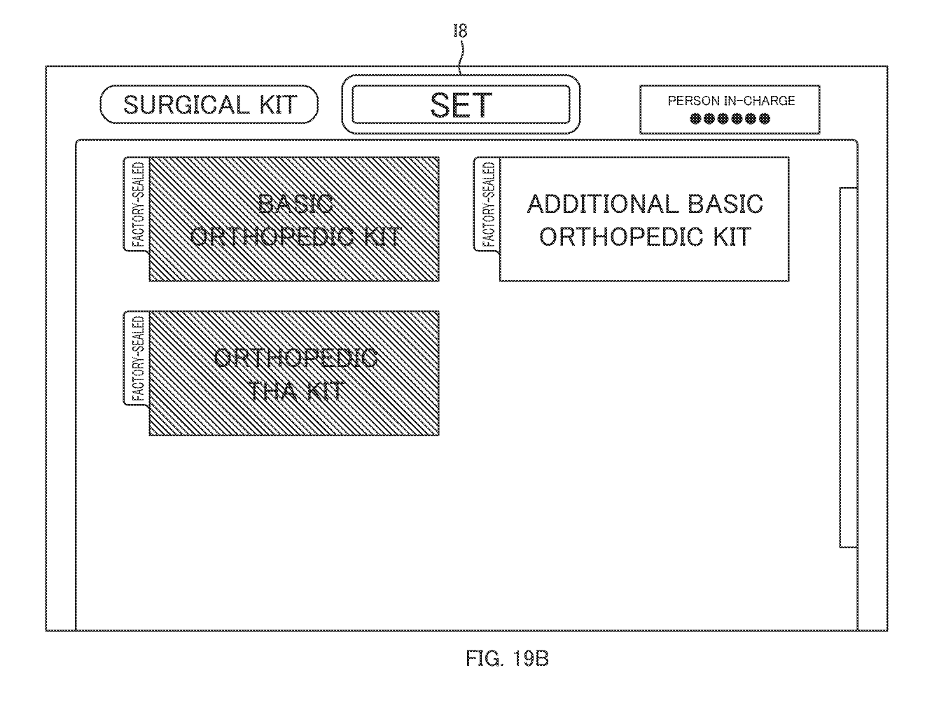

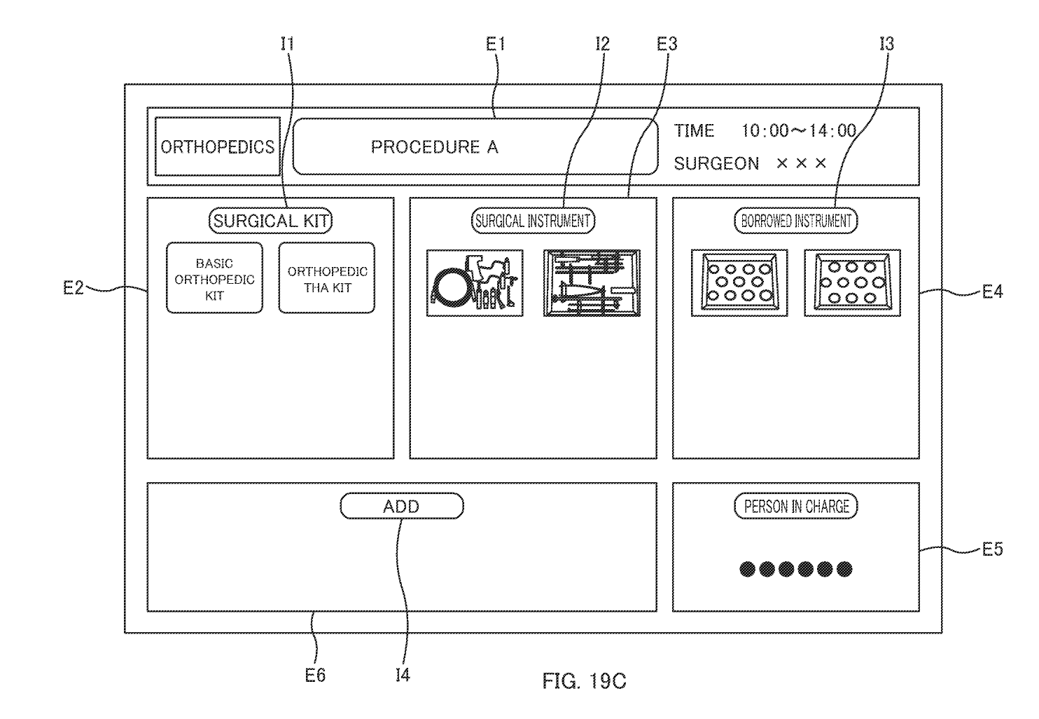

Now, referring to FIGS. 19A to 22D, the deleting and adding operations performed on the terminal P1 for the medical instrument related to the procedure A applied to the surgery α on the patient X are described. FIGS. 19A to 22D show screens displayed on the display 40. It is assumed that the operator enters various inputs (selections) via the operator 50.

First, an example of deleting the “additional basic orthopedic kit” from the surgical kit displayed on the screen shown in FIG. 15 is described.

In the top screen, the operator selects an icon I1 for the surgical kit. In response to this selection, the medical instrument register 70c switches the screen to a setting screen to set medical instruments included in the surgical kit (see FIG. 19A).

The operator selects only the necessary medical instrument(s) among a plurality of medical instruments (see FIG. 19B; in which the selected medical instruments are shown by hatching). After the necessary medical instruments are selected, the operator selects a “SET” icon I8. The medical instrument register 70c newly creates data for the procedure Aa obtained by deleting the “additional basic orthopedic kit” in the procedure A and allows it to be stored in the to-be-used instrument register 60c. Furthermore, the medical instrument register 70c updates the information in the medical procedure storage 60a. In this case, the display 40 displays a screen of a list of the surgical kit from which the “additional basic orthopedic kit” has been deleted (see FIG. 19C). It should be noted that, for the deletion of a medical instrument from the surgical instrument and the borrowed instrument, when the medical instrument displayed on the setting screen is an image rather than in a text format, a method similar to the above can be used.

Next, for the addition of a medical instrument, an example of adding a “one-piece scalpel handle” to the surgical kit displayed on the top screen is described.

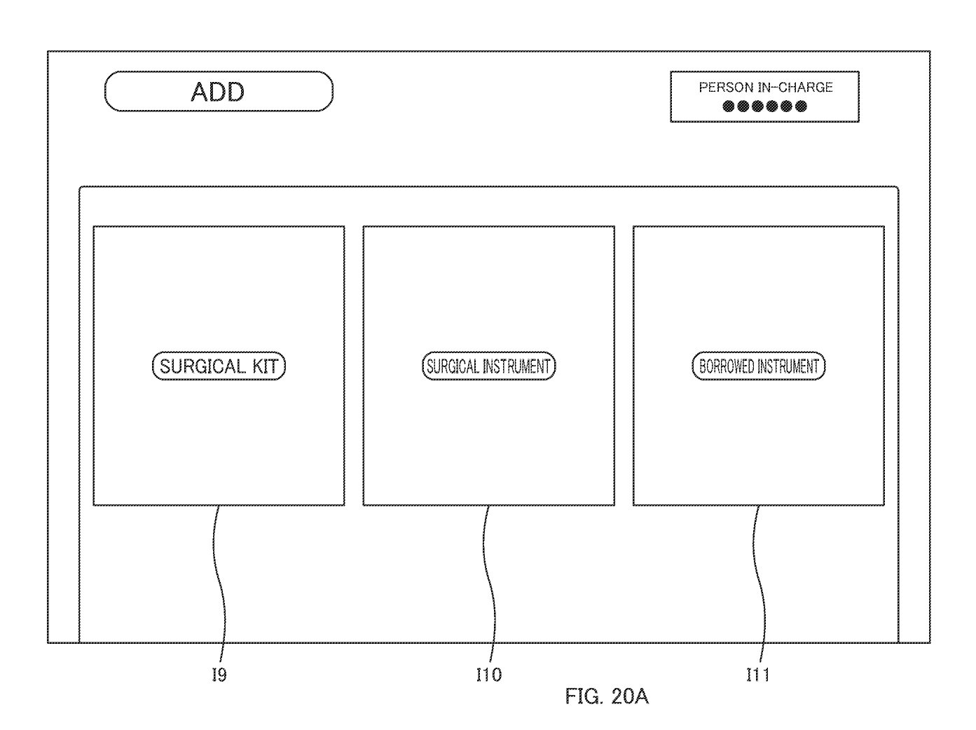

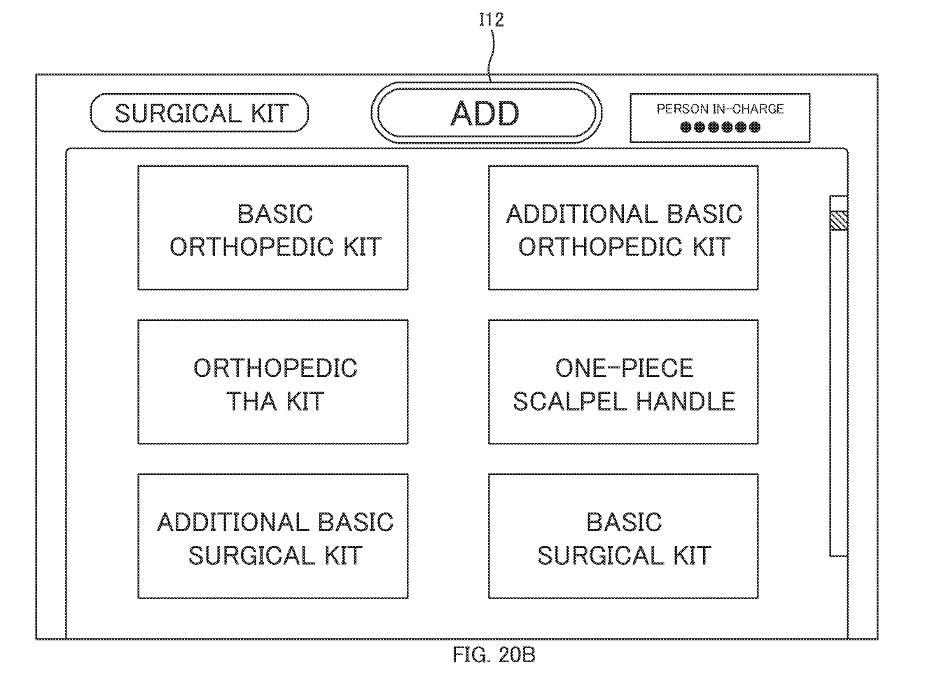

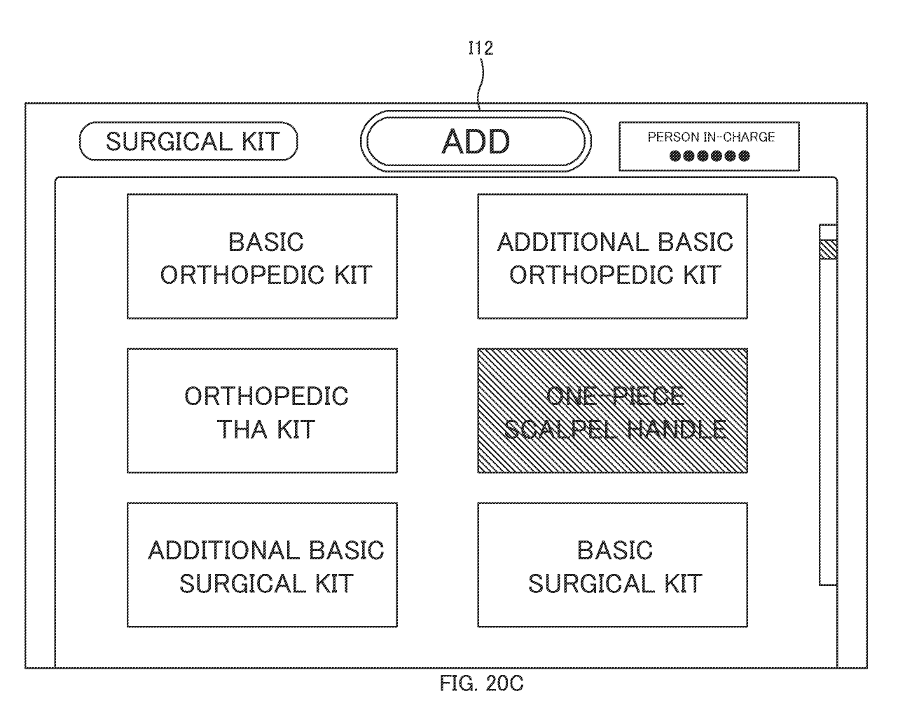

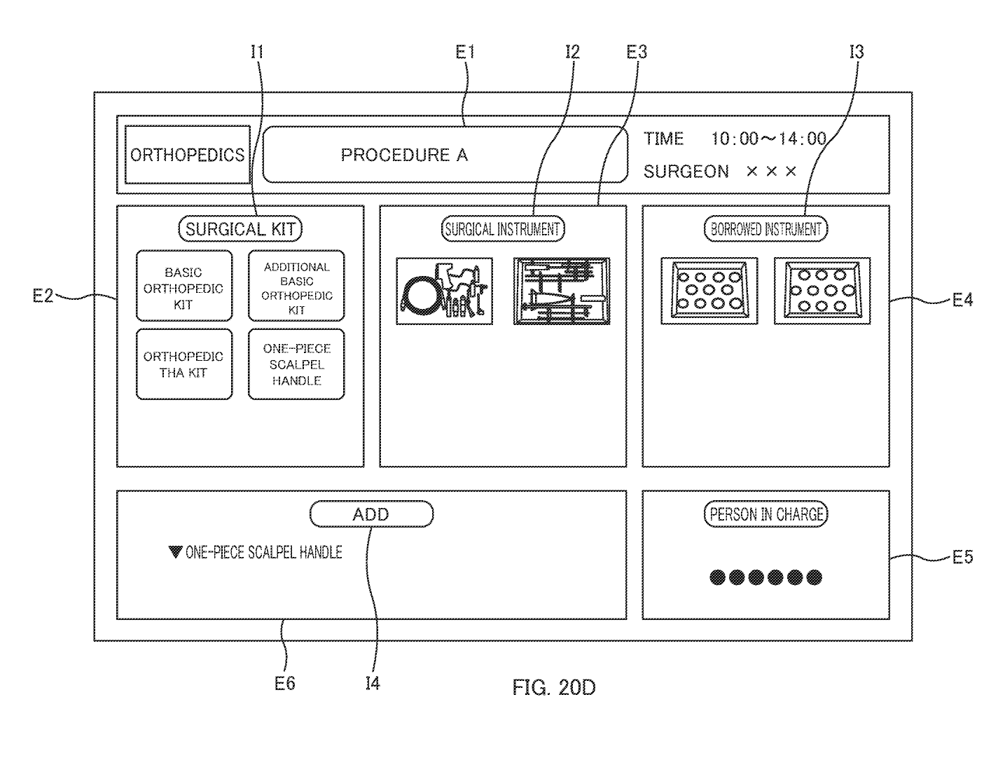

In the top screen, the operator selects an “add” icon I4. In response to this selection, the medical instrument register 70c switches the screen to an addition screen to add a medical instrument (see FIG. 20A). Class icons (icons I9 to I11) are displayed in the addition screen.

In the addition screen shown in FIG. 20A, the operator selects an icon I9 (an icon corresponding to the surgical kit). The medical instrument register 70c switches the screen to a selection screen to select a medical instrument to be added to the surgical kit (see FIG. 20B).

The operator selects a medical instrument to be added among a plurality of medical instruments (see FIG. 20C; in which the selected medical instrument is shown by hatching). After the medical instrument to be added is selected, the operator selects an “add” icon I12. The medical instrument register 70c newly creates data for the procedure Aaa obtained by adding the “one-piece scalpel handle” to the procedure A and allows it to be stored in the to-be-used instrument register 60c. Furthermore, the medical instrument register 70c updates the information in the medical procedure storage 60a. In this case, the display 40 displays a screen of a list of the surgical kit with “one-piece scalpel handle” added (see FIG. 20D). Furthermore, the name of the added medical instrument is displayed in an addition area E6 of the display 40 (see FIG. 20D). In this way, by allowing the name of the added medical instrument to be displayed separately, the operator can easily know the added medical instrument.

Furthermore, for the addition of a medical instrument, an example of adding a new medical instrument to the borrowed instrument is described.

As to the borrowed instrument that has been stored in the medical instrument storage 60b beforehand, the addition can be made in the manner described above. On the other hand, since medical instruments that are considered as the borrowed instrument are those on loan from a manufacturer or personal belongings of doctors, sufficient information management is not performed (not stored in the medical instrument storage 60b beforehand) in some cases, unlike the medical instruments that belong to the surgical kit and the surgical instrument. Now, an example of adding such a borrowed instrument as a new medical instrument is described.

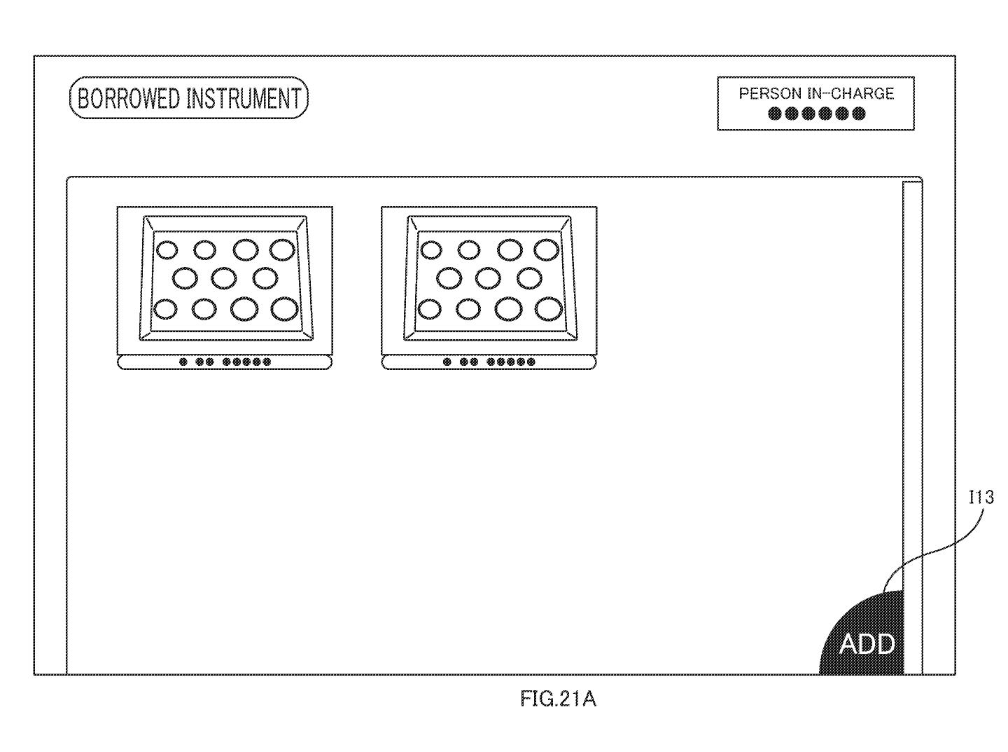

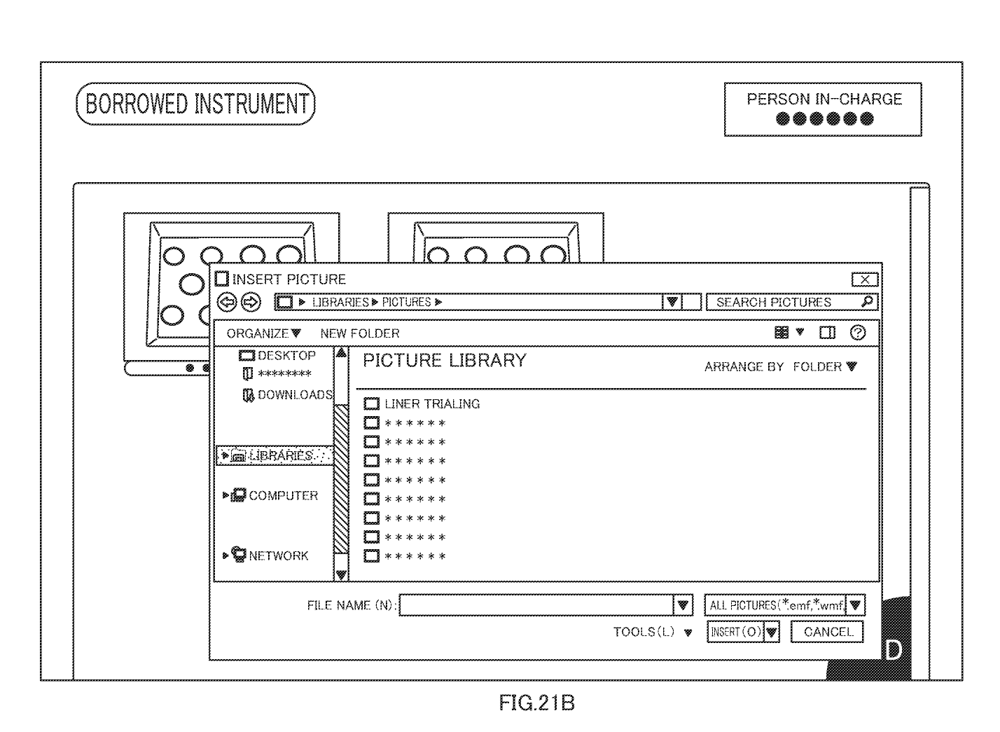

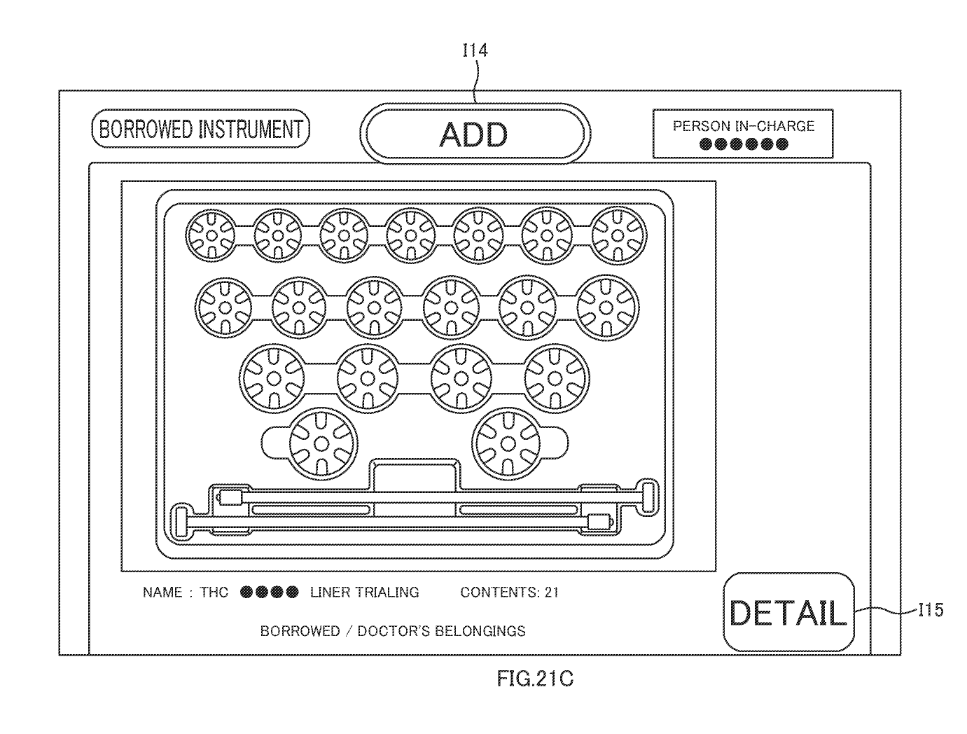

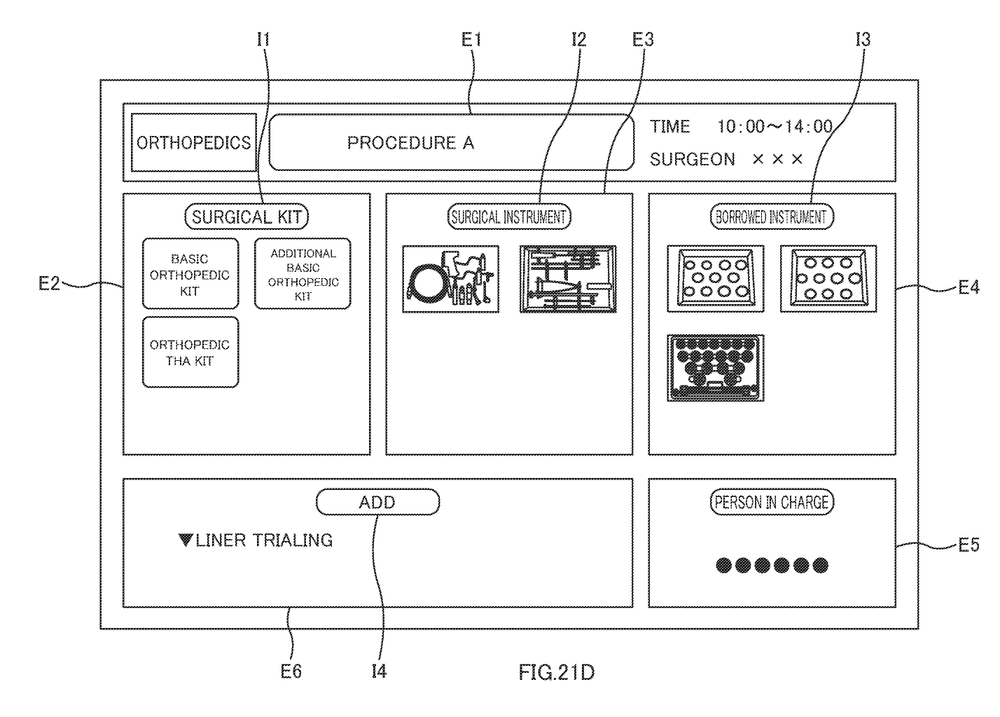

In the screen shown in FIG. 20A, when the operator selects an icon I11 (an icon corresponding to the borrowed instrument), the medical instrument register 70c switches the screen to an addition screen to add a new medical instrument to the borrowed instrument (see FIG. 21A). An “add” icon I13 is displayed in the addition screen.

When the operator selects the “add” icon I13, a list of image data related to the borrowed instrument is displayed in a text format (see FIG. 21B). The operator selects a desired image data (in this example, an image data of a liner trialing) from the list. It should be noted that the image data of the borrowed instrument that are displayed as a list are photographed beforehand using an image-pickup device and stored in the storage 60. Alternatively, an image data obtained by photographing a medical instrument to be added on site may be used rather than selecting a desired one from the stored image data.

Based on the selected image data, the medical instrument register 70c allows the image of the medical instrument to be displayed on the display 40 (see FIG. 21C). When the operator selects an “add” icon I14, the medical instrument register 70c allows the information of the liner trialing to be stored in the medical instrument storage 60b and newly creates data for the procedure Aaaa obtained by adding the “liner trialing” to the procedure A and allows it to be stored in the to-be-used instrument register 60c. Furthermore, the medical instrument register 70c updates the information in the medical procedure storage 60a. In this case, the display 40 displays a screen of a list of the borrowed instrument with “liner trialing” added (see FIG. 21D). Furthermore, the name of the added medical instrument is displayed in an addition area E6 of the display 40 (see FIG. 21D).

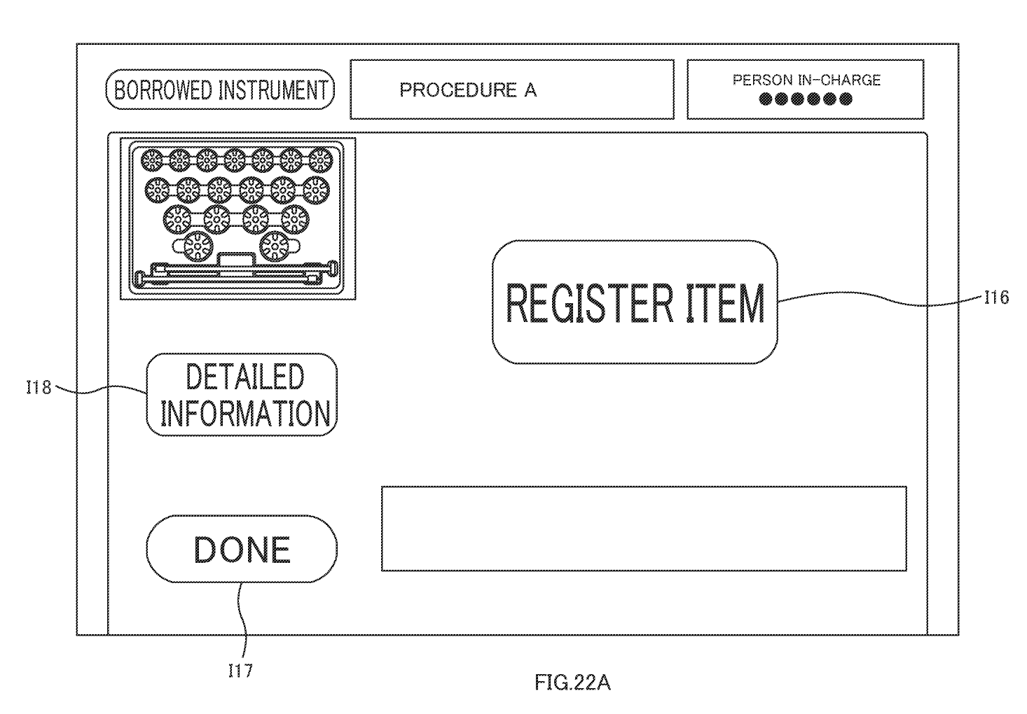

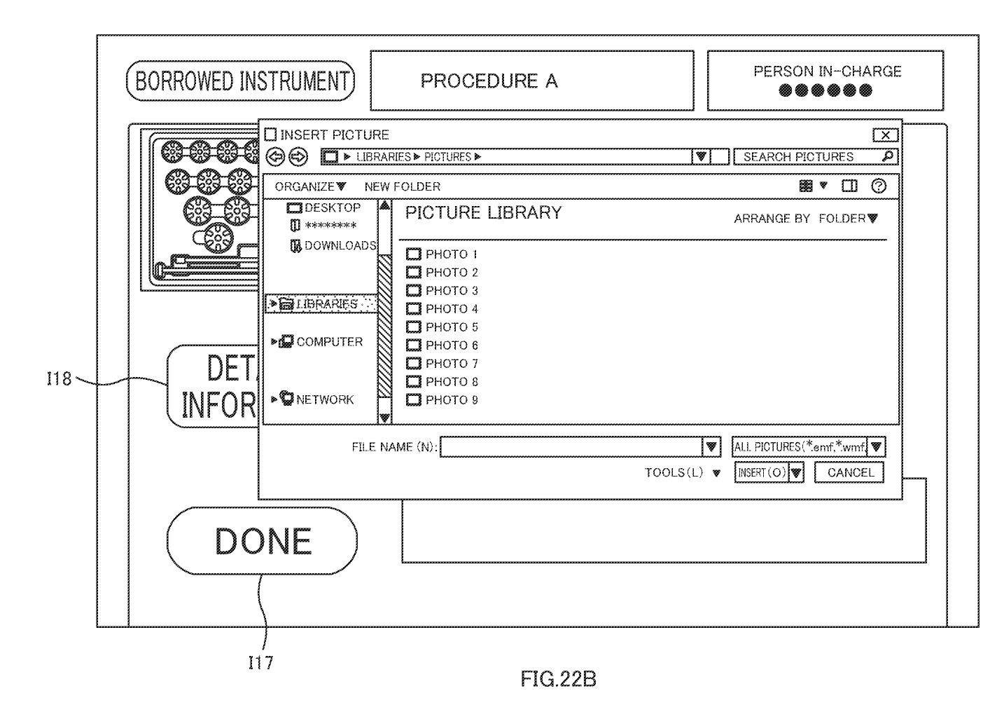

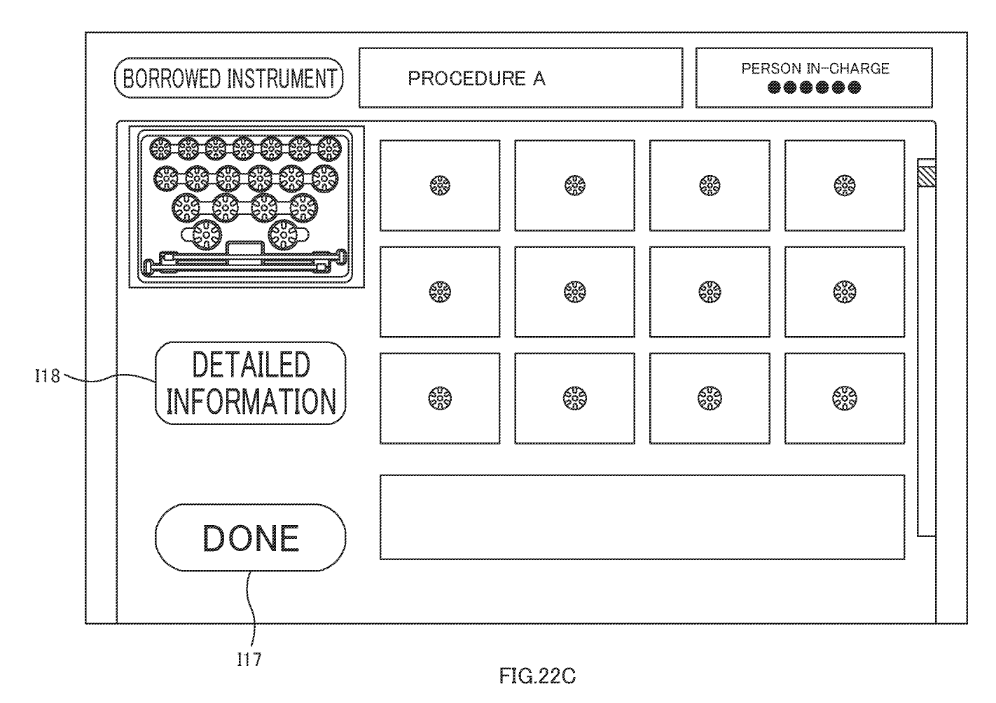

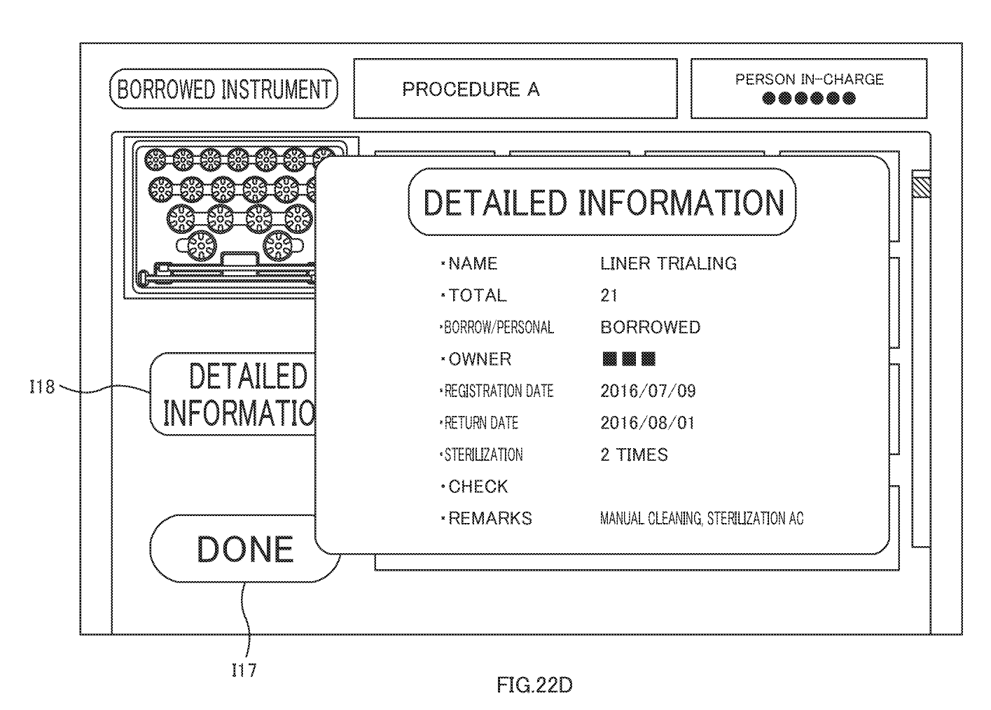

Furthermore, as the liner trialing shown in FIG. 21C, the added medical instrument may contain two or more instruments. In this case, it is possible to register the individual instruments when the medical instrument is added. In this case, the operator selects a “detail” icon I15 shown in FIG. 21C. The medical instrument register 70c allows an item registration screen to be displayed (see FIG. 22A). When the operator selects a “register item” icon I16, a list of image data related to the items of the liner trialing is displayed (see FIG. 22B). The operator selects a desired image data from the list. The image data of each item may be stored in the storage 60 beforehand or photographed on site as in the case of the image data of the medical instrument.

The medical instrument register 70c allows the image of the item to be displayed along with the image of the liner trialing on the display 40, based on the selected image data (see FIG. 22C). When the operator selects a “done” icon I17, the item is registered to the liner trialing. This data is stored in the medical instrument storage 60b.

Furthermore, when the operator selects a “detailed information” icon I18, the medical instrument register 70c allows a window for entering detailed information regarding the liner trialing to be displayed (see FIG. 22D). The operator can enter detailed information in a window. When the “done” icon I17 is selected, the medical instrument register 70c allows the detailed information to be stored in the medical instrument storage 60b with association to the medical instrument.

It should be noted that, when a medical instrument is added, the operator performs checking (count operation) as described above before releasing it.

Furthermore, it would be possible that an unexpected event (such as a failure of an instrument) occurs and it becomes necessary to add a medical instrument during a medical procedure. The system 1 of this preferred embodiment can also manage the addition of a new medical instrument that is performed during a medical procedure. In this case, the medical instrument register 70c registers the new medical instrument in the same manner as described above. Furthermore, the first count log generator 70b is incremented for the new medical instrument according to a command input and creates a count log for the new medical instrument.

The terminal P2 of this preferred embodiment functions as a device to perform counting of the individual instruments included in a medical instrument that is collected after a certain medical procedure and allowing count logs before and after a certain medical procedure to be displayed. The terminal P2 includes a communicator 80, a storage 110, and a controller 120, and is connected to a display 90 and an operator 100.

The communicator 30 exchanges data with the server S and the terminal P1 via the network N. The display 90 displays, for example, images of medical instruments and a list of individual instruments. The display 90 and the terminal P2 may be one unit. The operator 100 is an input or interface such as a mouse. Alternatively, the display 90 of a touch-panel screen type may double as the operator 100. An operator enters commands to the terminal P2 via the operator 100. The operator 100 and the terminal P2 may be one unit.

The storage 110 is a large-capacity storage that stores various kinds of data. As shown in FIG. 10, in this preferred embodiment, a medical procedure storage 110a, a medical instrument storage 110b, a used instrument storage 110c, a display layout storage 110d are portions of a storage region of the storage 110.

In the present preferred embodiment, each storage stores data that are identical to those stored in the storage of the terminal P1. When the data in the terminal P1 has been changed (for example, a medical instrument has been added or deleted for a certain procedure), the terminal P2 receives the changed data indirectly through the server S or directly from the terminal P1 and updates the stored data.

The controller 120 includes a CPU and a memory (which are not shown). The CPU achieves different kinds of control functions by executing an operating program stored in the memory. The memory is a storage that stores a program or programs executed by the CPU or temporarily stores various pieces of information upon execution of the program(s). The controller 120 detects signals (command inputs) from the operator 120 and executes corresponding processing operations.

The controller 120 according to the present preferred embodiment defines and functions as a second display operator 120a and a second count log generator 120b.

The second display operator 120a allows medical instruments used for a certain medical procedure to be displayed and, when one of the medical instruments is selected, allows a second count screen to be displayed which is used for performing the counting of individual instruments included in the one of the medical instruments. The second display operator 120a is an example of the “display operator” and the second count screen is an example of the “count screen.”

Furthermore, the second display operator 120a allows count logs before and after a certain medical procedure to be displayed on the display 90. The second display operator 120a retrieves the latest count log for a certain medical procedure from the count logs stored in the storage 10 of the server S and allows it to be displayed on the display 90 (details are described below).

The second count log generator 120b performs counting according to a command input and creates a count log. The count log (the count log after a certain medical procedure) is related to a medical procedure ID and is stored in, for example, the storage 110. The second count log generator 120b is an example of the “count log generator.”

Furthermore, the second count log generator 120b of the present preferred embodiment can store user information to specify the operator who has performed the counting (e.g., the name of the operator, an operator ID) in the storage 110 along with the count log.

Now, as a specific example, an example of displaying, at the terminal P2, count logs before and after the surgery α for the medical instruments used for the surgery α on the patient X is described. It is assumed that the operator enters various inputs (selections) via the operator 50.

First, when collecting medical instruments from the operating room where the surgery α was performed, the operator checks the collected medical instruments.

The check of the collected medical instruments (the display and count operations at the terminal P2) is identical to the check of the medical instruments at the terminal P1; therefore, a detailed description will be omitted. It should be noted that, in the terminal P2, the second display operator 120a performs operations that are similar to those performed by the first display operator 70a, and the second count log generator 120b performs operations that are similar to those performed by the first count log generator 70b.

Thereafter, the operator checks whether all of the medical instruments that were released are complete (whether they come back). In the present preferred embodiment, the operator performs the checking by comparing the count logs before and after the surgery α. When performing this checking, the second display operator 120a allows, for example, a screen similar to the one shown in FIG. 15 to be displayed on the display 90 as a screen for checking.

Now, for example, if the operator selects the “basic orthopedic kit,” the second display operator 120a retrieves a count log after the collection of the “basic orthopedic kit” from the storage 110 and allows it to be displayed on the display 90. On the other hand, the second display operator 120a makes an inquiry to the server S to have a count log before the surgery α for the “basic orthopedic kit.” The server S reads the count log before the surgery α for the “basic orthopedic kit” from the count logs stored in the storage 10 and send it to the terminal P2. The second display operator 120a allows the count log received from the server S to be displayed on the display 90. The count logs before and after the surgery α may be arranged side by side in one screen or may be switched to display. The operator can know whether no instruments are lost before and after the surgery by checking the count logs before and after the surgery α with his eyes.

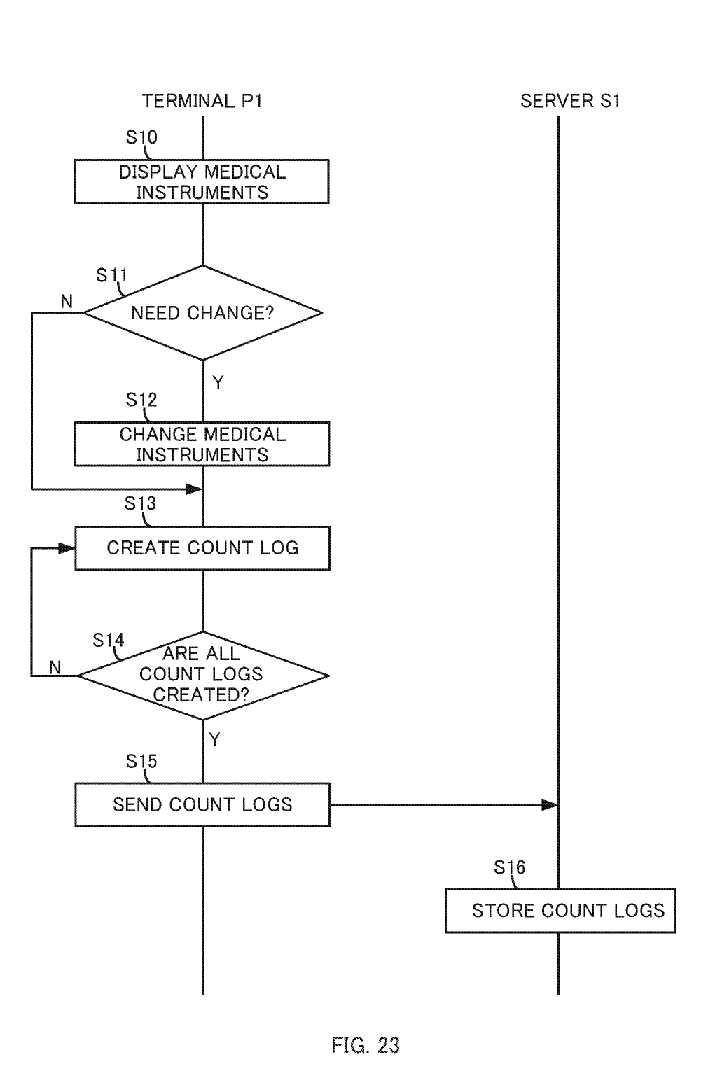

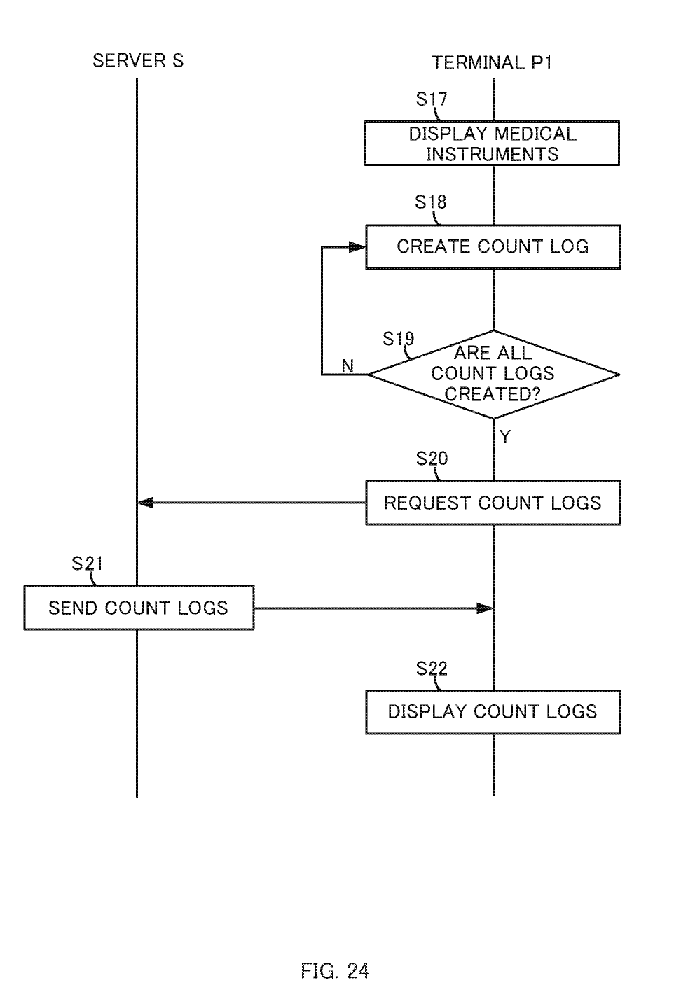

Referring to FIGS. 23 and 24, operations in the system 1 according to the present preferred embodiment are described. Specifically, an example that the operator compares the count logs before and after the surgery α is described. FIG. 23 is a flow chart showing an operation performed in the system 1 before the surgery α. FIG. 24 is a flow chart showing an operation performed in the system 1 after the surgery α.

Before releasing one or more medical instruments to be used for the surgery α, the operator checks whether the medical instruments required for the surgery α are complete.

In this case, the first display operator 70a retrieves the medical instruments used for the surgery α from the medical instrument storage 60b and allows them to be displayed on the display 40 (display medical instruments; S10).

If a medical instrument displayed at S10 is changed (addition or deletion) (Y at S11), the medical instrument register 70c performs the addition or deletion of the medical instrument (change medical instruments; S12).

Subsequently, the first count log generator 70b is incremented for individual instruments included in the medical instrument according to a command input from the operator and creates a count log (create count log; S13).

When the counting of individual instruments of all medical instruments has been completed and count logs have been created (Y at S14), the first count log generator 70b sends the count logs to the server S (send count logs; S15).

The server S stores the count logs transmitted at S15 in the storage 10 (store count logs; S16).

After the surgery α, the operator checks the collected medical instruments.

First, the second display operator 120a retrieves the medical instruments used for the surgery α from the medical instrument storage 60b and allows it to be displayed on the display 90 (display medical instruments; S17).

Then, the second count log generator 120b is incremented for the individual instruments included in the medical instrument according to a command input from the operator and creates a count log (create count log; S18).

When the counting of the individual instruments of all medical instruments has been completed and the count log has been created (Y at S19), the operator checks whether all medical instruments released before the surgery α come back.

In this case, the second display operator 120a requests the server S to send the count log performed before the surgery in the storage 10 (request count log; S20).

The server S sends the count log stored at S16 to the terminal P2 (send count log; S21).

The second display operator 120a displays the count log obtained at S15 and the count log sent at S21 on the display 90 so that they can be compared to each other (display count logs; S22). The operator can refer to the display at S22 and check, for example, whether all released medical instruments are collected.

As described above, the system 1 according to the present preferred embodiment makes it possible to allow the count logs before and after a certain medical procedure to be displayed. Therefore, the operator can determine, after a medical procedure, whether any medical instruments are lost by comparing the count logs before and after a certain medical procedure with his eyes. That is, the system 1 according to the present preferred embodiment makes it possible to accurately manage medical instruments before and after medical procedures.

### Third Preferred Embodiment

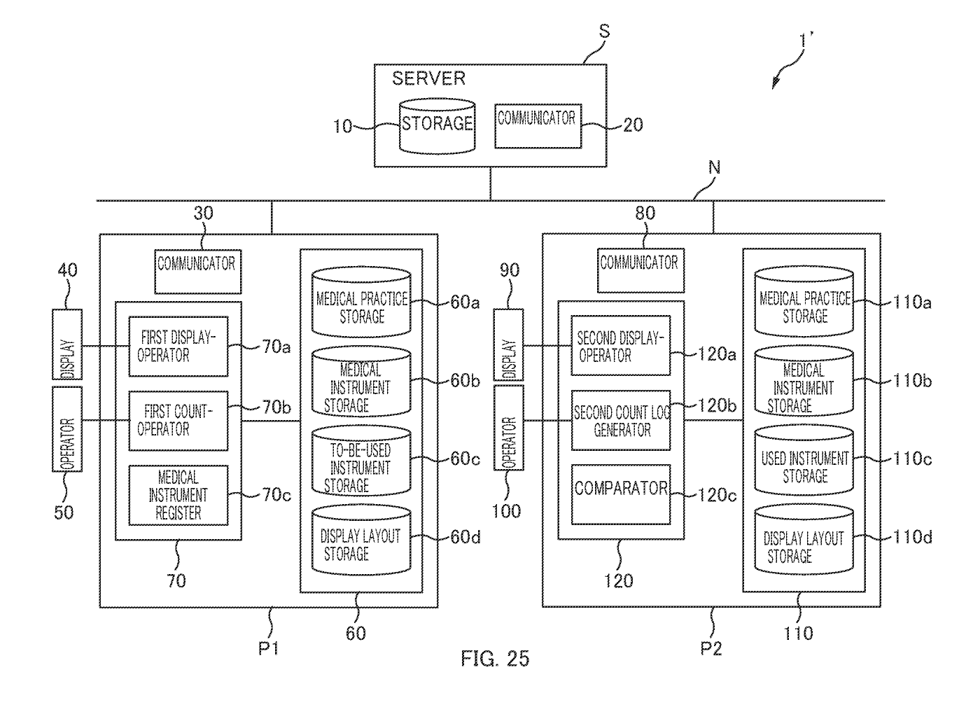

Next, referring to FIG. 25, a computer-aided management system for medical instruments according to a third preferred embodiment of the present invention (hereinafter, also referred to as “a system 1′”) is described.

In the second preferred embodiment, an example in which count logs before and after a certain medical procedure are allowed to be displayed and an operator checks them with his eyes is described. On the other hand, when many medical instruments are used for a medical procedure, it is troublesome to visually check them and mistakes in checking can occur. In this preferred embodiment, a configuration with which checking of count logs before and after a certain medical procedure can be performed more easily. Detailed description of the same components as the second preferred embodiment may be omitted.

The terminal P2 of the present preferred embodiment defines and functions as a device for performing counting of individual instruments included in the medical instrument after a certain medical procedure or comparing count logs before and after a certain medical procedure. As shown in FIG. 25, the controller 120 according to this preferred embodiment functions as the second display operator 120a, the second count log generator 120b, and a comparator 120c.

The comparator 120c compares count logs before and after a certain medical procedure.

For example, it is assumed that the second count log generator 120b performed counting of the individual instruments after the surgery α and a count log C-2 has been obtained. The second count log generator 120b supplies the count log C-2 thus obtained to the comparator 120c.

On the other hand, the comparator 120c requests the server S to send a count log C-1 before the surgery α. The server S retrieves the count log C-1 from the storage 10 and sends it to the terminal P2. The comparator 120c compares the individual medical instruments for each medical instrument material based on the count logs C-1 and C-2. The comparison is performed as to whether the number of the medical instruments and the number of the individual instruments in the count logs coincide by checking matches and mismatches of lists or images.

The comparator 120c allows the comparison result to be displayed on the display 90. For example, when the comparison results match, it allows a text message of “all collected” to be displayed on the display 90. On the other hand, one or more instruments are missing, the comparator 120c allows names of the missing instruments or images of the missing instruments and their numbers to be displayed on the display 90. When disposable products or implants are included, the count logs differ from each other before and after the medical procedure. Thus, one or more missing instruments are displayed. The operator, however, can easily know that the missing items are disposable products or implants by viewing the name or a photograph of the instrument displayed on the display 90.

It should be noted that the present preferred embodiment is described as an example in which the terminal has a comparator but is not limited thereto. For example, the comparator may be provided in the server S. In such a case, the terminal P1 sends the count log C-1 before the medical procedure to the server S. Furthermore, the terminal P2 sends the count log C-2 after the medical procedure to the server S. The comparator in the server S compares these count logs and sends the comparison result to the terminal P2. The second display operator 120a allows the received comparison results to be displayed on the display 90. With such a configuration, the operator can check whether any instruments are missing after medical procedures.

As described above, the system 1′ according to the present preferred embodiment can compare count logs before and after a certain medical procedure. Therefore, by checking the comparison result, the operator can know more easily whether any instruments are missing after medical procedures. That is, the system 1′ according to the present preferred embodiment allows more accurate management of medical instruments before and after medical procedures.

Although the configuration having the server S and a plurality of terminals (the terminal P1, the terminal P2) is described in the aforementioned preferred embodiments, the computer-aided management system for medical instruments of the invention according to this preferred embodiment is not limited thereto. For example, it is possible to provide a computer-aided management system for medical instruments with one terminal and a server. In such a case, a display operator in one terminal defines and functions both as the first display operator 70a and the second display operator 120a, and a count log generator in one terminal serves both as the first count log generator 70b and the second count log generator 120b. Alternatively, without interfacing the server S, only one terminal (or a plurality of terminals) can display and compare count logs before and after medical procedures. In such a case, one terminal (a plurality of terminals) is equivalent to the computer-aided management system for medical instruments.

In the above preferred embodiment, an example of comparing count logs based on the counting separately performed before and after a medical procedure is described. On the other hand, it is also possible to use the count log obtained before the medical procedure for counting of medical instruments performed after a certain medical procedure.

The first count log generator in this example creates a count log and allows the created count log to be stored in a storage (for example, the storage 10 of the server S), as in the preferred embodiment described above.

On the other hand, the second display operator in this example retrieves the count log from the storage 10 according to a command input and allows the second count screen to be displayed on the display 90. That is, the instruments etc. displayed on the second count screen in this case reflect the count result obtained at the first count log generator.

The operator enters a command while comparing the second count screen and the collected medical instrument(s) that is/are kept close at hand. The second count log generator in this example performs counting of the individual instruments on the second count screen according to the command input and creates a count log after the medical procedure.

With such a configuration, for example, in the case where one or more instruments has a missing item in the counting before the medical procedure, this information can be directly indicated on the second count screen. Therefore, the operator can easily know the situation of missing items and the like.

### Fourth Preferred Embodiment

A fourth preferred embodiment of the present invention provides non-transitory computer-readable storage media and programs.

When a medical procedure such as a surgery or therapy is performed on a patient, it is common to use two or more medical instruments.

In performing a medical procedure, a method of managing medical instruments using a database to efficiently prepare the medical instruments to be used has been known. For example, JP-A-2002-369787 discloses a storage that stores records of instruments used for medical procedures in a database.

Meanwhile, it is necessary to accurately manage medical instruments used for medical procedures. For this management, it is common to manage them by classifying medical instruments into item categories, and using sheets of paper on which item category names and quantities of medical instruments are written as being related to each other for each item category. However, even in the same hospital, the item category names may be different for each medical department or each doctor although it is the same item category. In addition, rules of which items belong to which item category may sometimes be inconsistent in a hospital. Therefore, if all necessary items are prepared for each medical department and doctor, more than the necessary number of medical instruments are kept as inventory in the entire hospital.

This preferred embodiment makes it possible to accurately manage classifications of medical instruments for each item category.

A non-transitory computer-readable storage medium has stored thereon a classification table as a data, the classification table being used to classify medical instruments into item categories, each medical instrument being assigned with a unique identifier that is unique to each medical instrument, in which the classification table includes an item category identifier that is unique to each item category and is common to medical instruments that belong to the item categories; and a unique identifier for the medical instruments that belong to the item categories, the unique identifier being related to the item category identifier is disclosed.

According to this preferred embodiment, it is possible to accurately manage classification of medical instruments for each item category using a computer.

In the descriptions of the specification and the drawings that follow, at least the following features are disclosed.

A non-transitory computer-readable storage medium that has stored thereon a classification table as a data, the classification table being used to classify medical instruments into item categories, each medical instrument being assigned with a unique identifier that is unique to each medical instrument, in which the classification table includes an item category identifier that is unique to each item category and is common to medical instruments that belong to the item categories; and a unique identifier for the medical instruments that belong to the item categories, the unique identifier being related to the item category identifier is disclosed.

In the storage medium mentioned above, the storage medium has stored a classification table in which a common item category identifier is related to unique identifiers of medical instruments that belong to the same item category. Thus, when a computer looks up this classification table, it is possible to specify an item category identifier from a unique identifier or vice versa.

Accordingly, it becomes possible to accurately manage classifications of medical instruments for each item category using a computer.

Preferably, the storage medium further stores a set information table for grouping the medical instruments into sets, in which the set information table includes a set identifier that is unique to each set; and an item category identifier of the item category that belongs to a set, the item category identifier being related to the set identifier.

With this, even when the medical instruments that belong to the same item category are interchanged between different sets, a computer can recognize that the medical instruments after the exchange also belong to the same item category by looking up the classification table and the set information table. Therefore, it becomes possible to accurately manage grouping and classification of medical instruments for each set and item category using a computer.

Preferably, the program causes a computer that can read the storage medium to execute a method including an acquisition processing to acquire a unique identifier assigned to a predetermined medical instrument; and a specifying, by looking up the classification table, processing to specify an item category identifier related to a unique identifier that is the same as the unique identifier acquired by the acquiring processing.

With the above, it is possible to make a computer recognize item category to which each medical instrument belongs.

Referring to the drawings, this preferred embodiment is described below. The preferred embodiment described below includes various features that are technically preferable for the purpose of implementing the present invention; therefore, the scope of the present invention is not limited to the following preferred embodiment and illustrated examples.

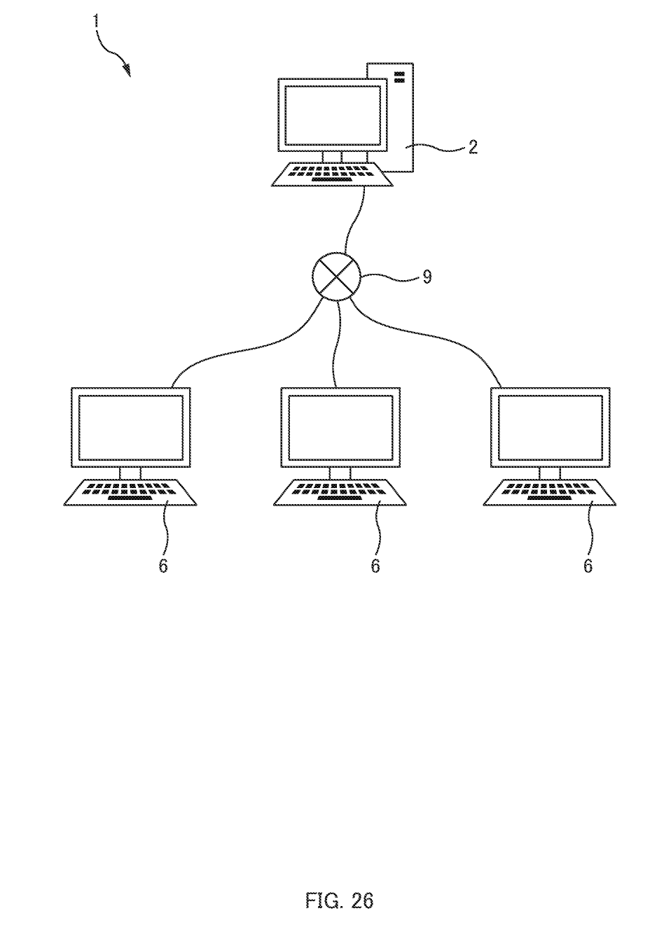

As shown in FIG. 26, a management system 1 includes a server system 2, a plurality of client systems 6, and a communication network 9. The client systems 6 can communicate with the server system 2 via the network 9.

Each client system 6 is a desktop type, laptop type or tablet type computer system. Each client system 6 may be a portable computer system or a computer system on a desk.

The server system 2 is a tower type, desktop type, rack mount type or blade type computer system. The communication network 9 is an intranet, a local area network (LAN), the Internet, or the like.

The management system 1 is a system for managing whether medical instruments that have been transferred (delivered) from a storage place to an operating place are used in a medical procedure. Specifically, the management system 1 is a system for summing up the number of times that the medical instruments were not used even though they had been transferred from the storage place to the operating place.

An operating place in a hospital refers to a place where surgery, medication, treatment, examination or other medical procedure is performed; for example, a treatment room, an operating room, a medical room or an examination room. The client systems 6 are provided at the operating place(s). The server system 2 can be installed in a hospital facility where the operating place is located or installed in a remote location such as a server provider and the like.

The medical instruments refer to instruments such as endoscopes, ultrasonic probes, pairs of forceps, pairs of scissors, scalpels, scalpel handles, cannulas, tweezers, retractors, scales, Sondes, elevators, raspas, suction tubes, rib spreaders, rib contractors, needle holders, syringes, metal balls, kidney dishes, cups, pins, mirrors, files, opening devices, Klemmes, handpieces, Elevatoriums, chisels, curettes, raspatories, mirrors, suture needles, rongeurs, water receivers, needles, spatulas, bougies, vent tubes, bone impactors, rongeurs, needle-nose pliers, hammers, goniometers, perforators, droppers, metal swabs, enemas, syringes and the like.

Each medical instrument is given a unique identifier. For example, medical instruments may be provided with a two-dimensional code, a one-dimensional code, a mark, a numerical value, a pattern or an image that represents a unique identifier or an RF tag storing information representing a unique identifier.

Medical instruments are classified into item categories, but a unique identifier for each item category is not attached to the medical instruments.

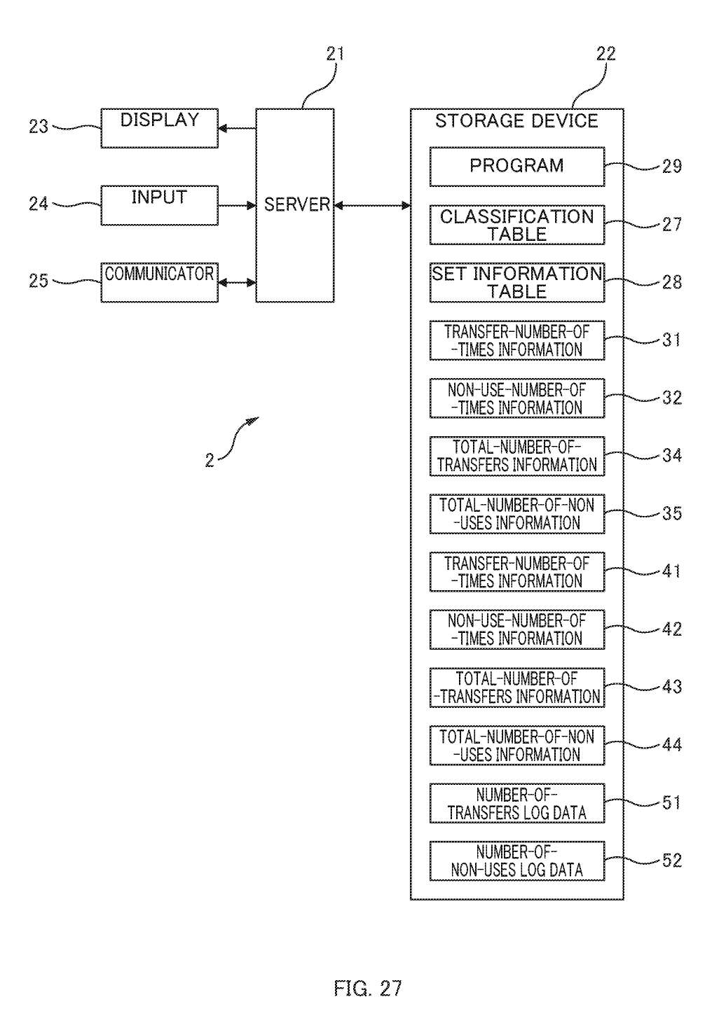

Any range can be defined for each item category. For example, medical instruments from different manufacturers (their applications and functions are the same) may be regarded as the same item category. In addition, the medical instruments for the same purpose having the same function may be regarded as a different item category when they are different in size, shape or color etc. Furthermore, the medical instruments belonging to the same item category may have different names. The item category classifications for medical instruments are defined by using classification tables 27 and 67 (see FIGS. 27, 28, and 35) described later.

Medical instruments to be transferred from a storage place to an operating place are divided into groups, and a collection of medical instruments constituting a group is called a set. That is, two or more medical instruments belong to the set. The medical instruments belonging to the set are delivered the operating place in a state of being gathered, for example, in a container (for example, a tray, a storage basket, a storage box, a storage bag or a storage container) or assembled.

Each set is given a unique set identifier. For example, a set may be provided with a two-dimensional code, a one-dimensional code, a mark, a numerical value, a pattern or an image that represents a set identifier or an RF tag storing data representing a unique identifier. Assigning a set identifier to a set means that a set identifier is attached to some or all of the medical instruments belonging to the set, that a set identifier is attached to a container in which the medical instruments belonging to the set are gathered, and that a set identifier is attached to a set of medical instruments belonging to the set, and the like.

In the storage place, the medical instruments may be stored after being divided as separate sets or may be stored after being divided into separate item categories. In the latter case, the operator picks out the medical instruments and puts them together before releasing them to an operating place.

Regardless of whether used or not, the medical instruments delivered to an operating place are returned to the storage place after being collected, cleaned and sterilized after the medical procedure. Thus, the medical instruments are reused.

As shown in FIG. 27, this server system 2 preferably includes a server 21, a storage 22, the display 23, an input 24, and a communicator 25

The server 21 is a computer including a CPU, a GPU, a ROM, a RAM, a bus, a hardware interface, and the like. The storage 22 is a storage such as a semiconductor memory, a hard disk drive, and the like that is readable and writable by the server 21. The display 23 is a display that performs screen display. The input 24 is an input such as a touch panel, a switch, a keyboard, a pointing device, an RFID reader, a one-dimensional code reader, and a two-dimensional code reader. The communicator 25 is a network adapter (for example, a network interface card, a wireless LAN slave).

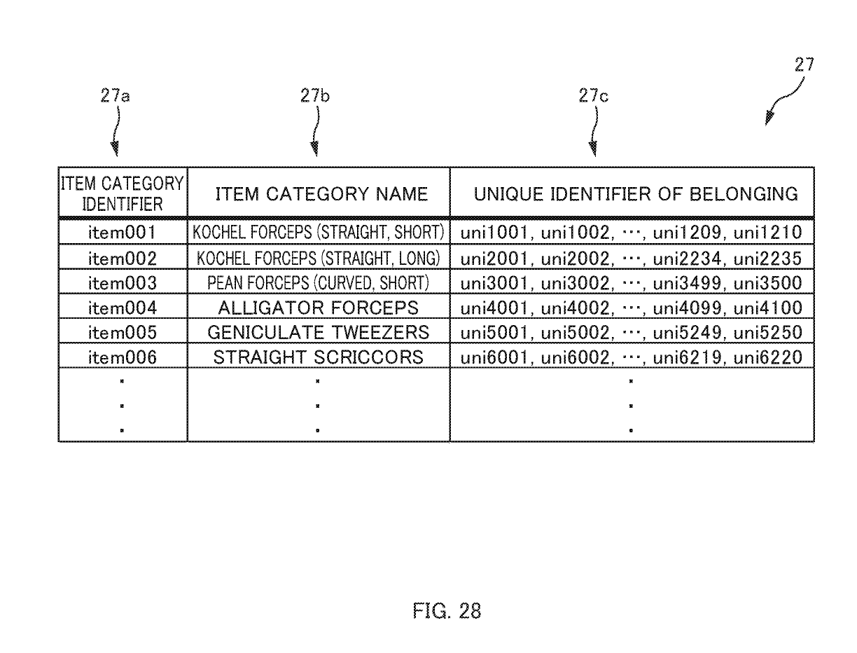

The storage 22 has stored thereon a classification table 27 for classifying medical instruments into item categories. FIG. 28 is a diagram showing an example of a configuration of the classification table 27. The classification table 27 includes item category identifiers 27a each of which is unique to an item category, pieces of item category name information 27b which are related to the item category identifiers 27a and each of which represents an item category name, and unique identifiers 27c which are related to the item category identifiers 27a for the medical instruments that belong to the item categories. Each item category identifier 27a is a common ID that is shared by all of the unique identifiers 27c related thereto. It means that medical instruments associated with a unique identifier that is the same as the unique identifier 27c belong to the item category of the item category identifier 27a related to the unique identifier 27c. It should be noted that, since the same medical instruments do not belong to different item categories, the same unique identifiers 27c are not related to different item category identifiers 27a.

When the server 21 refers to the classification table 27 as mentioned above, any item category identifier 27a can be specified from the unique identifier 27c and any unique identifier 27c can be specified from the item category identifier 27a. Accordingly, by using the server 21, it becomes possible to accurately manage classifications of instruments for each item category.

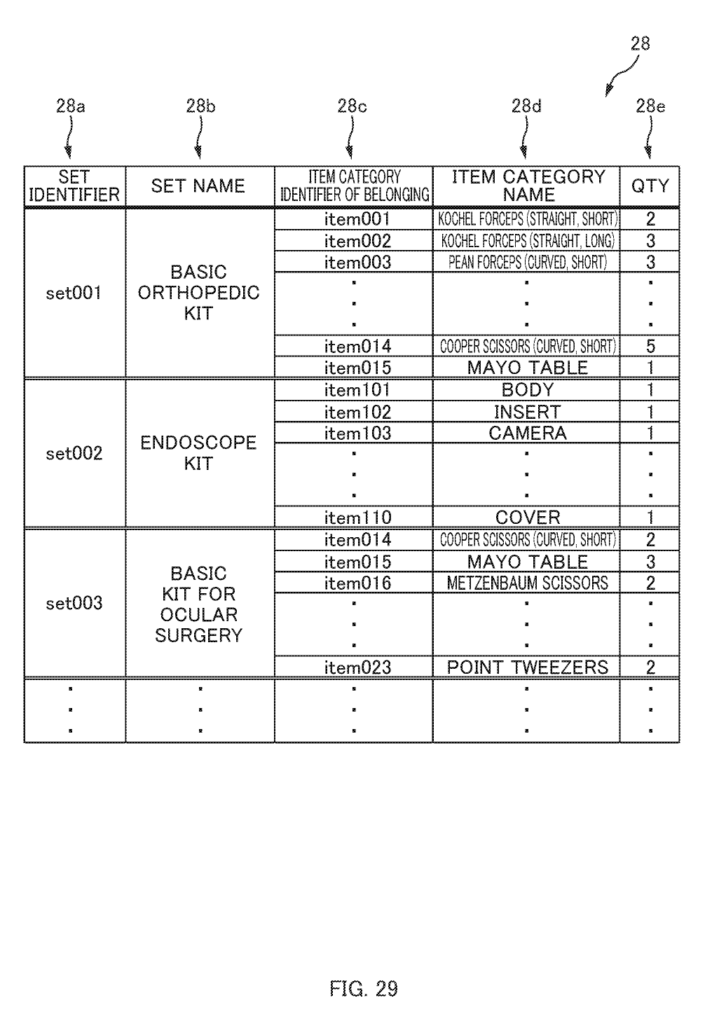

Furthermore, the storage 22 has stored thereon a set information table 28 that represents combinations of item categories that belong to sets. FIG. 29 a diagram showing an example of a configuration of the set information table 28. The set information table 28 includes set identifiers 28a unique to each set, pieces of set name information 28b which are related to the set identifiers 28a and each of which represents a set name, item category identifiers 28c which are related to the set identifier 28a for the item categories that belong to the sets, pieces of item category name information 28d which are related to the set identifiers 28a and the item category identifiers 28c and each of which represents an item category name, and pieces of quantity information 28e which are related to the set identifiers 28a and the item category identifiers 28c and each of which represents the number of medical instruments that belong to the sets and the item categories. It should be noted that, since the same item category may belong to different sets, the same item category identifier 28c may be related to different set identifiers 28a.

When the server 21 looks up the set information table 28 as described above, it is possible to specify the set identifier 28a from the item category identifier 28c or specify the item category identifier 28c from the set identifier 28a. Thus, it becomes possible to manage grouping of item categories for each set by using the server 21. Furthermore, the item category identifiers 28c are related to the set identifiers 28a in the set information table 28, and the unique identifiers 27c are related to the item category identifiers 27a in the classification table 27. Accordingly, even when the medical instruments that belong to the same item category are interchanged between different sets, the server 21 can recognize that the medical instruments after the exchange also belong to the same item category by looking up the classification table 27 and the set information table 28.

The storage 22 has stored thereon a program 29 that can be executed by the server 21.

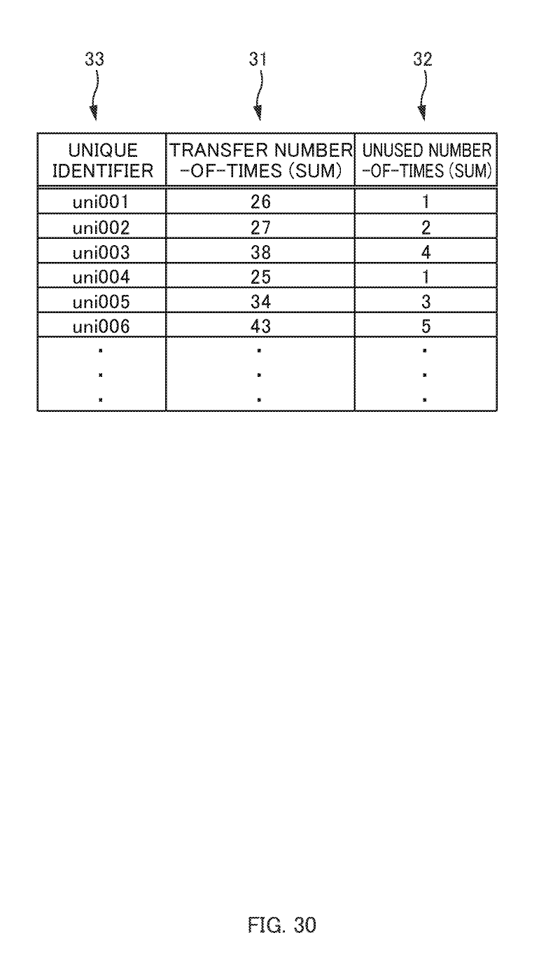

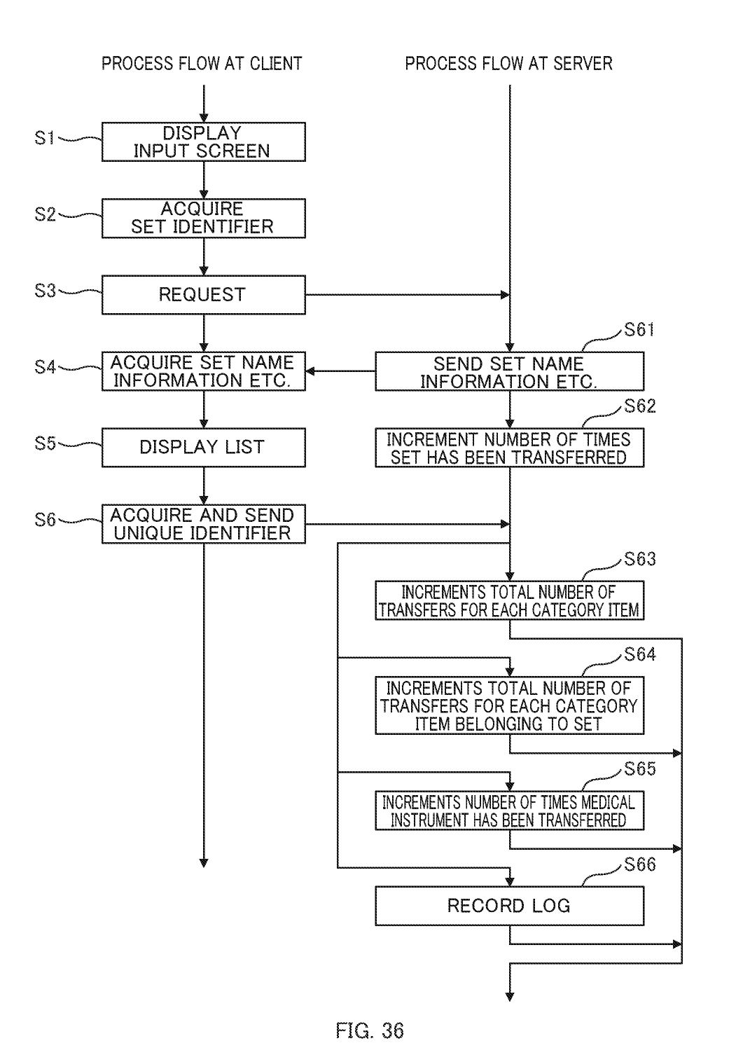

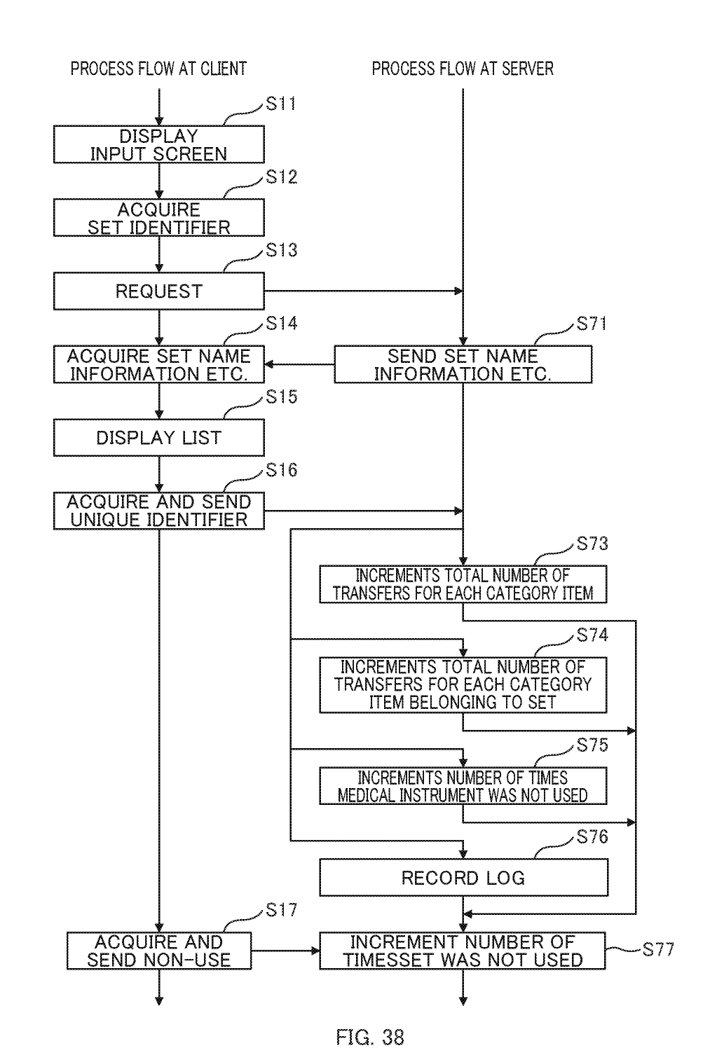

In the storage 22, transfer-number-of-times information 31 that represents the number of times each medical instrument has been transferred is stored and updated by an operation performed at step S65 described below (see FIG. 36). In the storage 22, non-use-number-of-times information 32 that represents the number of times each medical instrument was not used (the number of times it remained unused even though it had been transferred) is stored and updated by an operation performed at step S75 described below (see FIG. 38). Each of the transfer-number-of-times information 31 and the non-use-number-of-times information 32 is a current sum and the initial value for each of the transfer-number-of-times information 31 and the non-use-number-of-times information 32 is zero. As shown in FIG. 30, the transfer-number-of-times information 31 and the non-use-number-of-times information 32 are stored and updated in the storage 22 with relevance to a unique identifier 33 for each medical instrument.

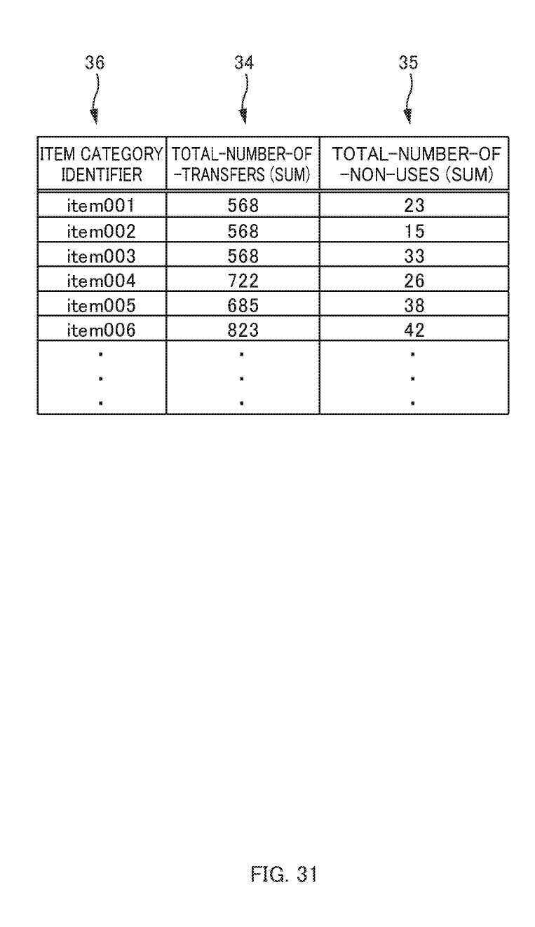

In the storage 22, total-number-of-transfers information 34 that represents the total number of times medical instruments for each item category has been transferred (total number of transferred medical instruments that belong to each item category) is stored and updated by an operation performed at step S63 described below (see FIG. 36). In the storage 22, total-number-of-non-uses information 35 that represents the total number of medical instruments that were not used for each item category (the total number of medical instruments that belong to item categories remained unused even though they had been transferred) is stored and updated by an operation performed at step S73 described below (see FIG. 38). Each of the total-number-of-transfers information 34 and the total-number-of-non-uses information 35 is a current sum and the initial value for each of the total-number-of-transfers information 34 and the total-number-of-non-uses information 35 is zero. As shown in FIG. 31, the total-number-of-transfers information 34 and the total-number-of-non-uses information 35 are stored and updated in the storage 22 with relevance to an item category identifier 36 that is unique to each item category.

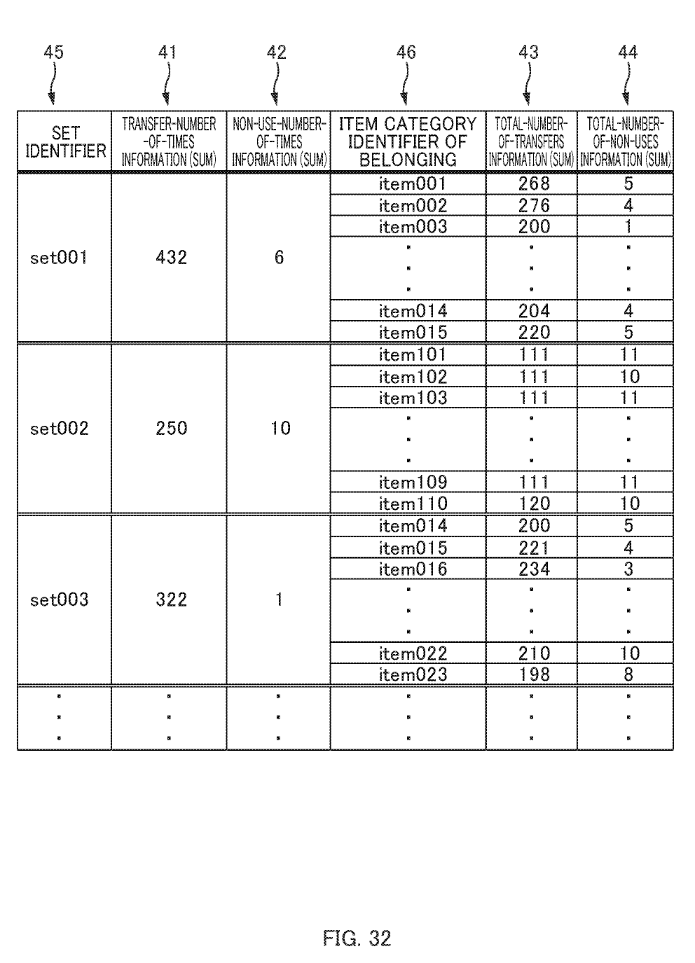

In the storage 22, transfer-number-of-times information 41 that represents the number of times each set has been transferred is stored and updated by an operation performed at step S62 described below (see FIG. 36). In the storage 22, non-use-number-of-times information 42 that represents the number of times each set was not used (the number of times it remained unused even though it had been transferred) is stored and updated by an operation performed at step S76 described below (see FIG. 38). Each of the transfer-number-of-times information 41 and the non-use-number-of-times information 42 is a current sum and the initial value for each of the transfer-number-of-times information 41 and the non-use-number-of-times information 42 is zero. As shown in FIG. 32, the transfer-number-of-times information 41 and the non-use-number-of-times information 42 are stored and updated in the storage 22 with relevance to a set identifier 45 for each set.

In the storage 22, total-number-of-transfers information 43 that represents the total number of transfers for each item category that belongs to a set is stored and updated by an operation performed at step S64 described below (see FIG. 36). In the storage 22, total-number-of-non-uses information 44 that represents the total number of medical instruments that were not used for each item category that belongs to a set (the total number of medical instruments that belong to a set and an item category remained unused even though they had been transferred) is stored and updated by an operation performed at step S74 described below (see FIG. 38). Each of the total-number-of-transfers information 43 and the total-number-of-non-uses information 44 is a current sum and the initial value for the total-number-of-non-uses information 44 is zero. As shown in FIG. 32, the total-number-of-transfers information 43 and the total-number-of-non-uses information 44 are stored and updated in the storage 22 with relevance to a set identifier 45 and an item category identifier 46.

The value of the total-number-of-transfers information 43 is the total number (sum) of the medical instruments belonging to an item category of the item category identifier 46 which have been transferred as a set with the set identifier 45. In contrast, the value of the total-number-of-transfers information 34 is the total number (sum) of the medical instruments belonging to an item category of the item category identifier 36 among all of the transferred sets have been transferred. Accordingly, the sum of the values of the total-number-of-transfers information 43 related to the same item category identifier 46 is equal to the sum of the values of the total-number-of-transfers information 34 related to the item category identifier 36 that is the same as the item category identifier 46. The same applies to the total-number-of-non-uses information 35 and the total-number-of-non-uses information 44. The sum of the values of the total-number-of-non-uses information 44 related to the same item category identifier 46 is equal to the sum of the values of the total-number-of-non-uses information 35 related to the item category identifier 36 that is the same as the item category identifier 46.

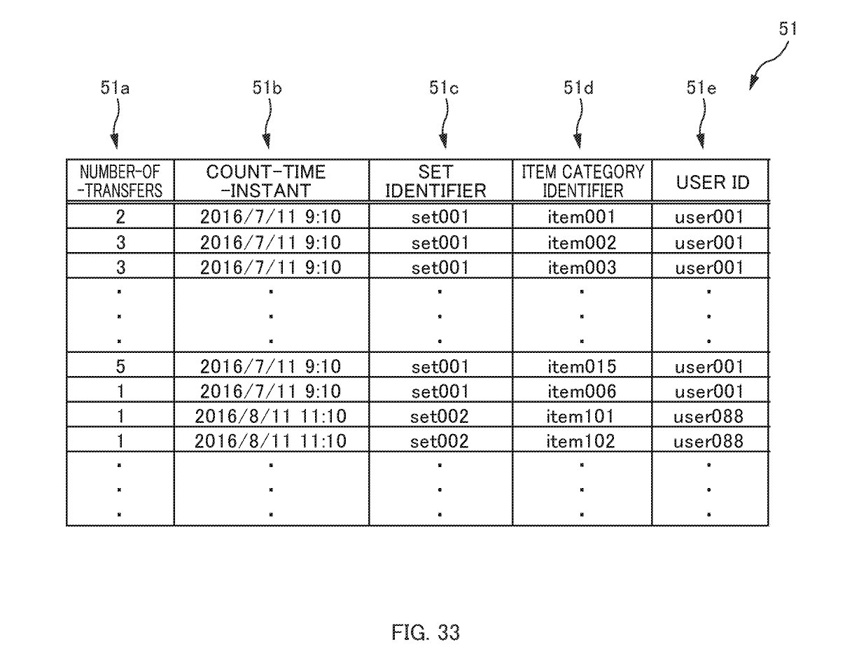

In the storage 22, a number-of-transfers log data 51 is stored by an operation performed at step S66 described below (see FIG. 36). As shown in FIG. 33, the number-of-transfers log data 51 is a relational representation of number-of-transfers information 51a that represents the number of medical instruments that have been transferred for each item category per a single transfer of a set, count-time-instant information 51b that represents the time instant at which the number of the transferred medical instruments was counted, one or more set identifiers 51c, one or more item category identifiers 51d, and one or more user IDs 51e. It should be noted that the time is in units of days, hours, minutes or seconds, or less than one second.

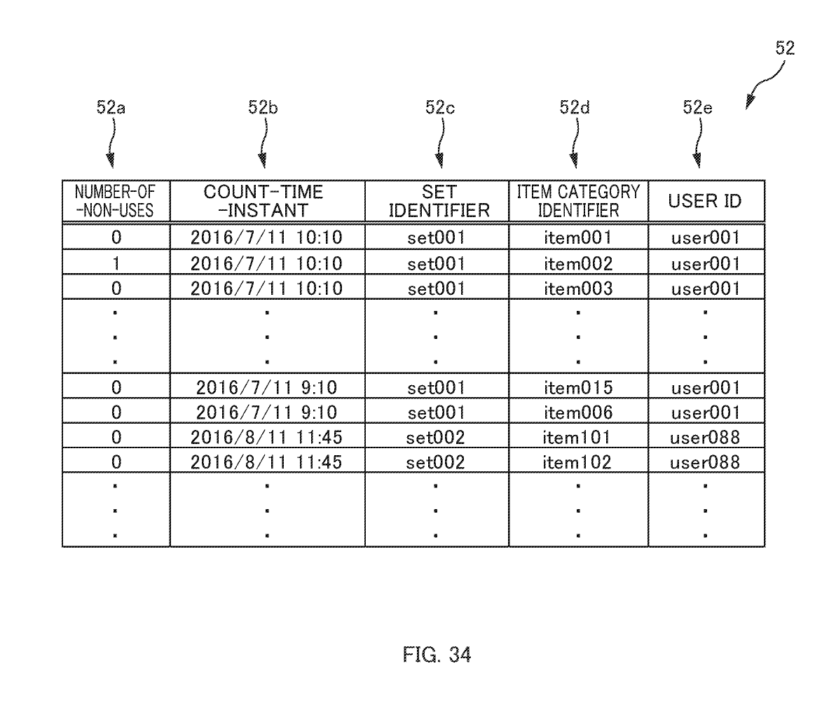

In the storage 22, number-of-non-uses log data 52 is stored by an operation performed at step S76 described below (see FIG. 38). As shown in FIG. 34, the number-of-non-uses log data 52 is a relational representation of number-of-non-uses information 52a that represents the number of unused medical instruments for each item category per a single transfer of a set, count-time-instant information 52b that represents the time instant at which the number of the unused medical instruments was counted, one or more set identifiers 52c, one or more item category identifiers 52d, and one or more user IDs 52e. It should be noted that the time is in units of days, hours, minutes or seconds, or less than seconds.

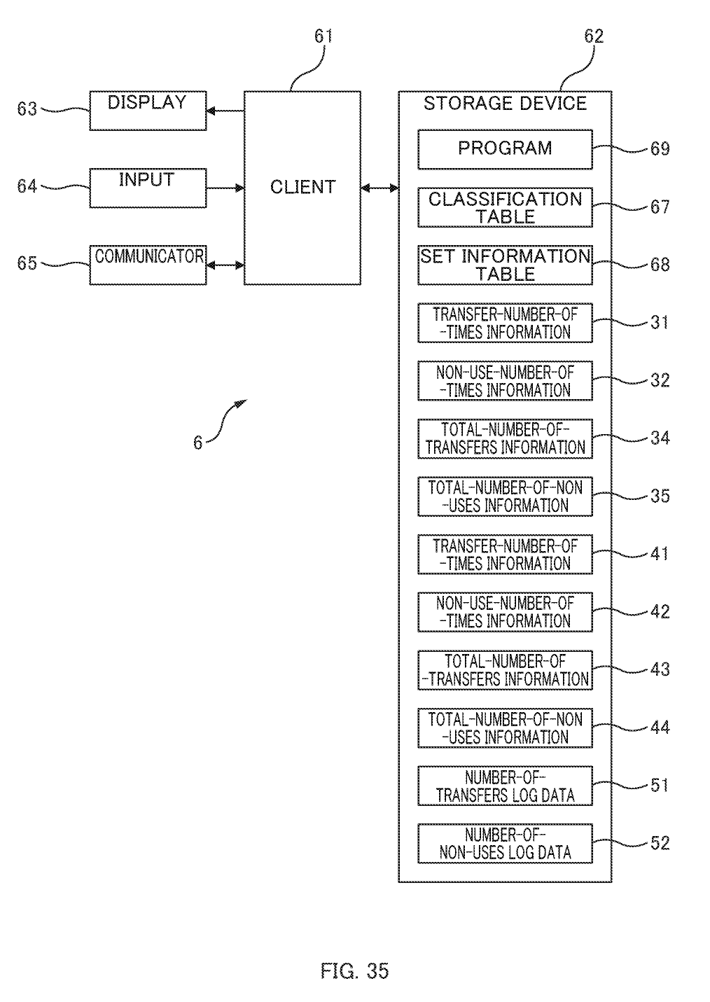

These pieces of information 31, 32, 34, 35, 41 to 44 and data 51 and 52 may be stored and updated in a storage 62 of a client system 6 (see FIG. 35) described below.

As shown in FIG. 35, a client system 6 preferably includes a client 61, a storage 62, a display 63, an input 64, and a communicator 65.

The client 61 is a computer preferably including a CPU, a GPU, a ROM, a RAM, a bus, a hardware interface, and the like. The storage 62 may be a semiconductor memory, a hard disk drive, and the like that is readable and writable by the client 61. The display 63 is a display that performs screen display. The input 64 is an input interface such as a touch panel, a switch, a keyboard, a pointing device, an RFID reader, a one-dimensional code reader, and a two-dimensional code reader. The communicator 65 is a network adapter (for example, a network interface card, a wireless LAN slave).

The storage 62 has stored thereon a program 69 that can be executed by the client 61.

Furthermore, in the storage 62, a classification table 67 and a set information table 68 are stored. The classification table 67 is in synchronism with the classification table 27 stored in the storage 22 of the server system 2 and the set information table 68 is in synchronism with the set information table 28 stored in the storage 22. Accordingly, the classification table 67 includes the item category identifier 27a, the item category name information 27b, and the unique identifier 27c which are related to each other, as in the case of the classification table 27 shown in FIG. 28. Furthermore, the set information table 68 includes the set identifier 28a, the set name information 28b, the item category identifier 28c, the item category name information 28d, and the quantity information 28e which are related to each other, as in the case of the set information table 28 shown in FIG. 29. Here, by the client 61 requesting to the server 21, the classification table 27 (or a difference between the classification table 67 and the classification table 27) is transferred from the server 21 to the client 61. This synchronizes the classification table 67 of the storage 62 and the classification table 27 of the storage 22. The same applies to the set information tables 28 and 68.

The function of this synchronization between the server 21 and the client 61 is achieved by the programs 29 and 69.

A method of using the management system 1 at the time of transferring medical instruments will be described. Besides, referring to FIG. 36, a flow of processing that the program 29 causes the server 21 to execute and a flow of processing that the program 69 causes the client 61 to execute in using the management system 1 are also described.

First, an operator releases a set from a storage place to an operating place. In the following description, a set transferred to an operating place is referred to as a given set, a medical instrument belonging to the given set is referred to as a given instrument, and an item category belonging to the given set is referred to as a given item category.

Next, the operator operates the input 64 of the client system 6 at the operating place and logs in to the management system 1 using his own user ID and password. Then, the client 61 causes the display 63 to display a set identifier input screen (set selection screen) (step S1).

Thereafter, the operator enters a set identifier for the given set via the input 64, and selects a name or a set identifier of the given set from several options. As a result, the client 61 gets the set identifier according to the input signal from the input 64 (step S2). As described above, the input 64 is a touch panel, a switch, a keyboard, a pointing device, an RFID reader, a one-dimensional code reader, and a two-dimensional code reader, or the like, and the set identifier is entered or selected by the operation depending on the kind of the input 64.

Next, the client 61 requests the server 21 to send information of the given set by sending the set identifier of the given set to the server 21 (step S3). Then, the server 21 looks up the set information table 28, retrieves the set name information 28b, the item category identifier 28c, the item category name information 28d, and the quantity information 28e which are related to the set identifier 28a that is the same as the set identifier of the given set, and sends them to the client 61 (step S61). On the other hand, the client 61 receives and acquires the set name information 28b, the item category identifier 28c, the item category name information 28d, and the quantity information 28e (step S4). It should be noted that the client 61 may look up the set information table 68 in the storage 62 without making a request as step S3 because the set information table 68 that is the same as the set information table 28 is synchronized with the storage 62. That is, the client 61 may retrieve the set name information 28b, the item category identifier 28c, the item category name information 28d, and the quantity information 28e which are related to the set identifier 28a that is the same as the set identifier of the given set, from the set information table 68 in the storage 62.

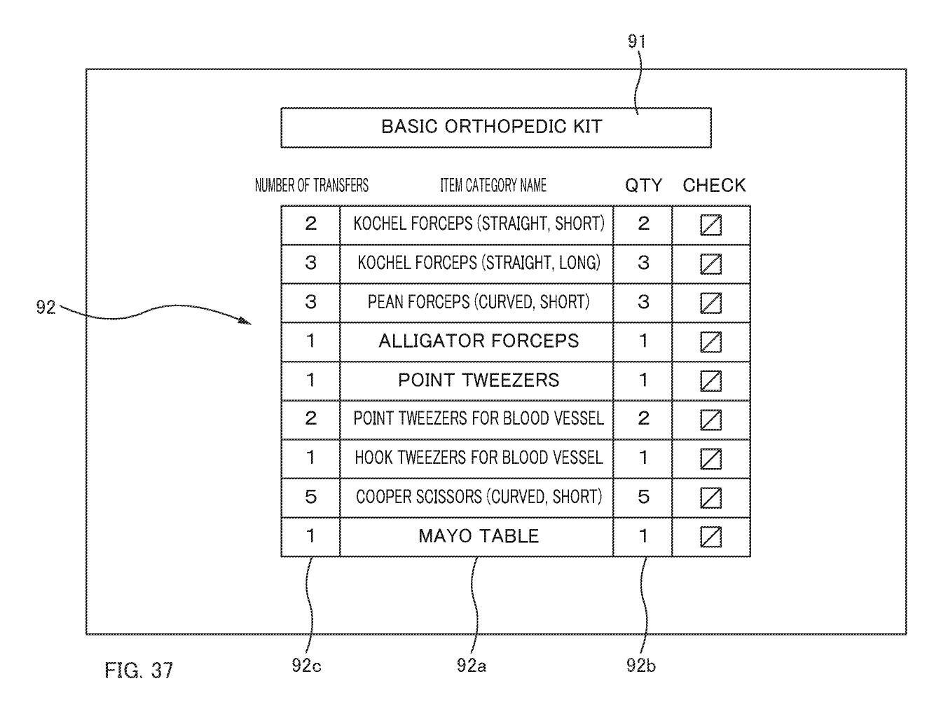

Next, the client 61 allows a screen as shown in FIG. 37 to be displayed on the display 63 according to the set name information 28b, the item category identifier 28c, the item category name information 28d, and the quantity information 28e which have been acquired (step S5). In a set name display area 91 on the screen, a set name according to the set name information 28b is displayed. In a list display area 92 on the screen, item category names according to the item category name information 28d and the quantities according to the quantity information 28e are displayed with relevance to each other. Specifically, item category names are displayed in an item category name display area 92a of the list display area 92 and the quantities are displayed in a quantity display area 92b on the right of the item category name display area 92a. Accordingly, the operator can check the contents of the transferred given set (such as the number of the medical instruments for each item category) while looking at the screen.

On the other hand, in the server system 2, the server 21 overwrites the transfer-number-of-times information 41 in the storage 22 by adding 1 to a value of the transfer-number-of-times information 41 related to the set identifier 45 that is the same as the set identifier of the given set (the set identifier received from the client 61) (see FIGS. 27 and 32) and relating that transfer-number-of-times information 41 to the set identifier 45 (step S62). Accordingly, the number of times the given set has been transferred is counted up and the value of the updated transfer-number-of-times information 41 is a sum. It should be noted that the operation at step S62 may be performed by the client 61. That is, after step S2, the client 61 may add 1 to the value of the transfer-number-of-times information 41 in the storage 62 related to the set identifier of the given set (see FIG. 35) and overwrite the transfer-number-of-times information 41 in the storage 62.

Then, the operator enters unique identifiers of given medical instruments one by one using the input 64. In response to this, the client 61 acquires unique identifiers according to the input signals from the input 64 and transmits the acquired unique identifiers to the server 21 with relevance to the set identifier of the given set (step S6).

The server 21 adds 1 to the value of the total-number-of-transfers information 34 related to one of the item category identifiers 36 (see FIGS. 27 and 31) every time when it receives a unique identifier from the client 61 (step S63). Specifically, every time when a unique identifier is received, the server 21 looks up the classification table 27 to specify the item category identifier 27a related to the unique identifier 27c that is the same as the received unique identifier and adds 1 to the value of the total-number-of-transfers information 34 related to the item category identifier 36 that is the same as that item category identifier 27a (step S63). Such operations are repeated until the last unique identifier is received. After the increment in response to the reception of the last unique identifier, the summed-up total-number-of-transfers information 34 is overwritten in the storage 22 by the server 21. Accordingly, the total number of transferred given medical instruments is counted up for each given item category, and the value of the updated total-number-of-transfers information 34 is a sum. It should be noted that the operation at step S63 may be performed by the client 61. That is, until the last unique identifier is acquired (see step S6), every time when a unique identifier is acquired, the client 61 may look up the classification table 67 to specify the item category identifier 27a related to the unique identifier 27c that is the same as the acquired unique identifier and add 1 to the value of the total-number-of-transfers information 34 in the storage 62 related to the item category identifier 36 that is the same as that item category identifier 27a (see FIG. 35).

Furthermore, the server 21 adds 1 to the value of the total-number-of-transfers information 43 related to one of the set identifiers 45 and the item category identifiers 46 (see FIGS. 27 and 32) every time when it receives a unique identifier from the client 61 (step S64). Specifically, every time when a unique identifier is received, the server 21 looks up the classification table 27 to specify the item category identifier 27a related to the unique identifier 27c that is the same as the received unique identifier and adds 1 to the value of the total-number-of-transfers information 43 related to the item category identifier 46 that is the same as that item category identifier 27a and the set identifier 45 that is the same as the received set identifier of the given set (step S64). Such operations are repeated until the last unique identifier is received. After the increment in response to the reception of the last unique identifier, the summed-up total-number-of-transfers information 43 is overwritten in the storage 22 by the server 21. Accordingly, the total number of transferred given medical instruments is counted up for each given item category, and the value of the updated total-number-of-transfers information 43 is a sum. It should be noted that the operation at step S64 may be performed by the client 61. That is, until the last unique identifier is acquired (see step S6), every time when a unique identifier is acquired, the client 61 may look up the classification table 67 to specify the item category identifier 27a related to the unique identifier 27c that is the same as the acquired unique identifier and add 1 to the value of the total-number-of-transfers information 43 related to the item category identifier 46 that is the same as that item category identifier 27a and the set identifier 45 that is the same as the set identifier of the given set (see FIG. 35).

Furthermore, the server 21 adds 1 to the value of the transfer-number-of-times information 31 related to the unique identifier 33 that is the same as the unique identifier received from the client 61 (see FIGS. 27 and 30) and overwrites the transfer-number-of-times information 31 in the storage 22 (step S65). Accordingly, the number of times given medical instruments belonging to the given set have been transferred is counted up and the value of the updated transfer-number-of-times information 31 is a sum. It should be noted that the operation at step S65 may be performed by the client 61. That is, after the acquisition of the unique identifier (step S6), the client 61 may add 1 to the value of the transfer-number-of-times information 31 related to the unique identifier 33 that is the same as the acquired unique identifier and overwrite the transfer-number-of-times information 31 in the storage 62.

As described above, every time when a unique identifier is received, the item category identifier 27a related to the unique identifier 27c that is the same as the received unique identifier is specified by looking up the classification table 27. The server 21 calculates the number of times it specifies the item category identifiers 27a from the reception of the first unique identifier to the reception of the last unique identifier, for each item category identifier 27a. Such a calculated value for each item category identifier 27a is equal to the number of the transferred medical instruments for each item category. The server 21 stores, in the storage 22, the number-of-transfers information 51a representing that calculated value, the count-time-instant information 51b representing the time instant at which the last unique identifier was received, the set identifier 51c that is the same as the set identifier received from the client 61, the item category identifier 51d that is the same as the specified item category identifier 27a, and the user ID 51e of the user who has logged in, with relevance to each other as shown in FIG. 8 (step S66). Here, the number-of-transfers information 51a etc. stored at step S66 is a log for a single processing shown in FIG. 36. The number-of-transfers information 51a etc. are accumulated by repeating the processing shown in FIG. 36, which are the number-of-transfers log data 51. It should be noted that the operation at step S66 may be performed by the client 61. That is, the client 61 may calculate the number of times the item category identifiers 27a are specified from the acquisition of the first unique identifier to the acquisition of the last unique identifier for each item category identifier 27a. In such a case, the client 61 stores, in the storage 62, the number-of-transfers information 51a, the count-time-instant information 51b, the set identifier 51c, the item category identifier 51d, and the user ID 51e, with relevance to each other as the number-of-transfers log data 51.

In the aforementioned fourth preferred embodiment, the values of the total-number-of-transfers information 34 and 43 for each given item category are incremented in the server 21 or the client 61 (see steps S63 and step S64) in response to the operator's successive entries of the unique identifiers of the given medical instruments using the input 64 (see step S6), and the number-of-transfers information 51a for each given item category is stored (see step S66).

On the other hand, the operator may count the number of given medical instruments for each given item category belonging to a given set and enter a counted value for each given item category using the input 64. For example, when the operator selects (e.g., clicks or taps) the item category name display area 92a depending on the number of times equal to the counted value or selects the item category name display area 92a once and then enters a numerical value of the counted value by operating the input 64 in a screen shown in FIG. 37, the entered counted value is displayed in a transferred-quantity display area 92c on the left of the item category name display area 92a. As a result, the client 61 acquires a counted value for each given item category according to the input signals from the input 64. The client 61 then sends, the acquired counted value to the server 21 with relevance to the item category identifier (which is the same as the item category identifier 28c acquired at step S4) and the set identifier (which is the same as the set identifier acquired at step S2). Thereafter, the server 21 adds the counted value to the value of the total-number-of-transfers information 34 (see FIGS. 27 and 31) related to the item category identifier 36 (see FIG. 31) that is the same as the item category identifier received from the client 61 and overwrites the total-number-of-transfers information 34 after the addition in the storage 22. Furthermore, the server 21 adds the counted value to the value of the total-number-of-transfers information 43 see FIGS. 27 and 32) related to the item category identifier 46 and the set identifier 45 (see FIG. 32) that are identical to the item category identifier and the set identifier received from the client 61 and overwrites the total-number-of-transfers information 43 after the addition in the storage 22.

Furthermore, the server 21 stores, in the storage 22, the number-of-transfers information 51a representing the counted value received from the client 61, the count-time-instant information 51b representing the time instant at which the counted value is received from the client 61, the set identifier 51c that is the same as the set identifier received from the client 61, the item category identifier 51d that is the same as the item category identifier received from the client 61, and the user ID 51e of the user who has logged in, with relevance to each other.

In the client system 6, the client 61 may add the counted value (input value from the input 64) to the value of the total-number-of-transfers information 34 (see FIG. 35) related to the item category identifier 36 that is the same as item category identifier related to the item category name in the item category name display area 92a and overwrite the total-number-of-transfers information 34 after the addition in the storage 62. Furthermore, the client 61 may add the counted value (input value from the input 64) to the value of the total-number-of-transfers information 43 (see FIG. 35) related to the item category identifier 46 and the set identifier 45 that are the same as the set identifier and the item category identifier identical to the ones in the display areas 91 and 92a and overwrite the total-number-of-transfers information 43 in the storage 62. Furthermore, the client 61 may store, in the storage 62, the number-of-transfers information 51a representing the counted value (input value from the input 64), the count-time-instant information 51b representing the time instant at which the counted value is entered, the set identifier 51c that is the same as the set identifier identical to the one in the set name display area 91, the item category identifier 51d that is the same as the item category identifier identical to the one in the item category name display area 92a, and the user ID 51e of the user who has logged in, with relevance to each other.

After medical instruments are delivered to an operating place, physicians and other members perform medical procedures (medical procedures such as surgery, medication, treatment, examination) using these medical instruments. A method of using the management system 1 after such medical procedures is described. Besides, referring to FIG. 38, a flow of processing that the program 29 causes the server 21 to execute and a flow of processing that the program 69 causes the client 61 to execute in using the management system 1 are also described.

First, an operator collects medical instruments in an operating place while sorting out used ones from unused ones. In the following description, a medical instrument that was or has been collected is referred to as a collected tool, an item category to which one or more collected tools belong is referred to as a collected item category, and a set to which one or more collected tools belong is referred to as a collected set. In addition, among the collected tools, a used medical instrument is referred to as a used tool and an unused medical instrument is referred to as an unused tool.

All medical instruments belonging to a certain set may be unused or used ones in some cases and some of medical members belonging to a certain set may be unused ones in other cases. Thus, the operator checks all of the medical instruments belonging to a set to determine whether they are unused or used ones.

Next, the operator operates the input 64 of the client system 6 at the operating place and logs in to the management system 1 using his own user ID and password. Then, the client 61 causes the display 63 to display a set identifier input screen (set selection screen) (step S11).

Thereafter, the operator enters a set identifier for the collected set via the input 64, and selects a set identifier or a name from several options. As a result, the client 61 gets the set identifier according to the input signal from the input 64 (step S12).

Next, the client 61 requests the server 21 to send information of the collected set by sending the set identifier of the collected set to the server 21 (step S13). Then, the server 21 looks up the set information table 28, retrieves the set name information 28b, the item category identifier 28c, the item category name information 28d, and the quantity information 28e which are related to the set identifier 28a that is the same as the set identifier of the collected set, and sends them to the client 61 (step S71). On the other hand, the client 61 receives and acquires the set name information 28b, the item category identifier 28c, the item category name information 28d, and the quantity information 28e (step S14). It should be noted that the client 61 may look up the set information table 68 in the storage 62 without making a request as step S13. That is, the client 61 may retrieve the set name information 28b, the item category identifier 28c, the item category name information 28d, and the quantity information 28e which are related to the set identifier 28a that is the same as the set identifier of the collected set, from the set information table 68 in the storage 62.

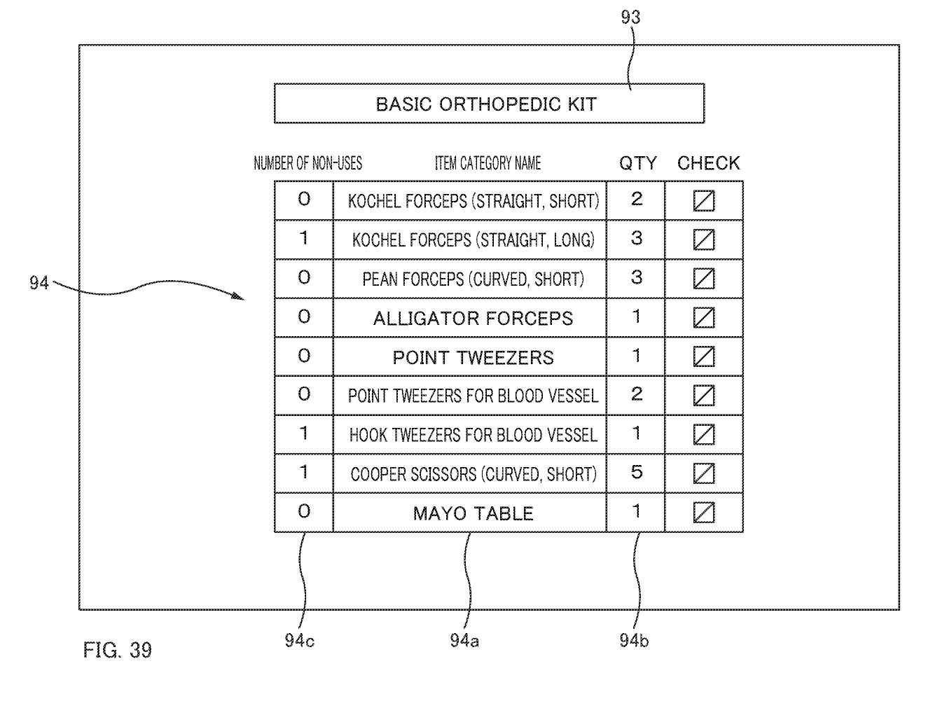

Next, the client 61 allows a screen as shown in FIG. 39 to be displayed on the display 63 according to the set name information 28b, the item category identifier 28c, the item category name information 28d, and the quantity information 28e which have been acquired (step S15). In a set name display area 93 on the screen, a set name according to the set name information 28b is displayed. In a list display area 94 on the screen, item category names according to the item category name information 28d and the quantities according to the quantity information 28e are displayed with relevance to each other. Specifically, item category names are displayed in an item category name display area 94a of the list display area 94 and the quantities are displayed in a quantity display area 94b on the right of the item category name display area 94a.

Then, the operator enters unique identifiers of unused medical instruments one by one using the input 64. In response to this, the client 61 acquires unique identifiers according to the input signals from the input 64 and transmits the acquired unique identifiers to the server 21 with relevance to the set identifier of the collected set (step S16).

The server 21 adds 1 to the value of the total-number-of-non-uses information 35 related to one of the item category identifiers 36 (see FIGS. 27 and 31) every time when it receives a unique identifier from the client 61 (step S73). Specifically, every time when a unique identifier is received, the server 21 looks up the classification table 27 to specify the item category identifier 27a related to the unique identifier 27c that is the same as the received unique identifier and adds 1 to the value of the total-number-of-non-uses information 35 related to the item category identifier 36 that is the same as that item category identifier 27a. Such operations are repeated until the last unique identifier is received. After the increment in response to the reception of the last unique identifier, the summed-up total-number-of-non-uses information 35 is overwritten in the storage 22 by the server 21. Accordingly, the total number of unused instruments is counted up for each collected item category, and the value of the updated total-number-of-non-uses information 35 is a sum. It should be noted that the operation at step S73 may be performed by the client 61. That is, until the last unique identifier is acquired (see step S16), every time when a unique identifier is acquired, the client 61 may look up the classification table 67 to add 1 to the value of the total-number-of-non-uses information 35 (see FIG. 35) of the item category identifier 36 that is the same as the item category identifier 27a related to the unique identifier 27c that is the same as the acquired unique identifier.

Furthermore, the server 21 adds 1 to the value of the total-number-of-non-uses information 44 related to one of the set identifiers 45 and the item category identifiers 46 (see FIGS. 27 and 32) every time when it receives a unique identifier from the client 61 (step S74). Specifically, every time when a unique identifier is received, the server 21 looks up the classification table 27 to specify the item category identifier 27a related to the unique identifier 27c that is the same as the received unique identifier and adds 1 to the value of the total-number-of-non-uses information 44 related to the item category identifier 46 that is the same as that item category identifier 27a and the set identifier 45 that is the same as the received set identifier of the given set (step S74). Such operations are repeated until the last unique identifier is received. After the increment in response to the reception of the last unique identifier, the summed-up total-number-of-non-uses information 44 is overwritten in the storage 22 by the server 21. Accordingly, the total number of the collected unused instruments is counted up for each collected item category belonging to a collected set, and the value of the updated total-number-of-non-uses information 44 is a sum. It should be noted that the operation at step S74 may be performed by the client 61. That is, until the last unique identifier is acquired (see step S16), every time when a unique identifier is acquired, the client 61 may look up the classification table 67 in the storage 62 to specify the item category identifier 27a related to the unique identifier 27c that is the same as the acquired unique identifier and add 1 to the value of the total-number-of-non-uses information 44 related to the item category identifier 46 that is the same as that item category identifier 27a and the set identifier 45 that is the same as the set identifier of the given set (see FIG. 35).

The server 21 adds 1 to the value of the non-use-number-of-times information 32 related to the unique identifier 33 that is the same as the unique identifier received from the client 61 (see FIG. 30) and overwrites the non-use-number-of-times information 32 in the storage 22 (step S75). Accordingly, the number of times the collected instruments were not used is counted up and the value of the updated non-use-number-of-times information 32 is a sum. It should be noted that the operation at step S75 may be performed by the client 61. That is, after the acquisition of the unique identifier (step S16), the client 61 may add 1 to the value of the non-use-number-of-times information 32 (see FIG. 35) related to the unique identifier 33 that is the same as the acquired unique identifier and overwrite the non-use-number-of-times information 32 in the storage 62.

As described above, every time when a unique identifier is received, the item category identifier 27a related to the unique identifier 27c that is the same as the received unique identifier is specified by looking up the classification table 27. The server 21 calculates the number of times it specifies the item category identifiers 27a from the reception of the first unique identifier to the reception of the last unique identifier, for each item category identifier 27a. Such a calculated value for each item category identifier 27a is equal to the number of the unused medical instruments for each item category. The server 21 stores, in the storage 22, the number-of-non-uses information 52a representing that calculated value, the count-time-instant information 52b representing the time instant at which the last unique identifier was received, the set identifier 52c that is the same as the set identifier received from the client 61, the item category identifier 51d that is the same as the specified item category identifier 27a, and the user ID 51e of the user who has logged in, with relevance to each other as shown in FIG. 34 (step S76). Here, the unused information 52a etc. stored at step S76 is a log for a single processing shown in FIG. 38. The number-of-non-uses information 52a etc. are accumulated by repeating the processing shown in FIG. 38, which are the number-of-non-uses log data 52. It should be noted that the operation at step S76 may be performed by the client 61. That is, the client 61 may calculate the number of times the item category identifiers 27a are specified from the acquisition of the first unique identifier to the acquisition of the last unique identifier for each item category identifier 27a. In such a case, the client 61 stores, in the storage 62, the number-of-non-uses information 52a, the count-time-instant information 52b, the set identifier 52c, the item category identifier 52d, and the user ID 52e, with relevance to each other as the number-of-non-uses log data 52.

Next, when all medical instruments belonging to a set are unused, the operator enters the set identifier of the collected set using the input 64 or selects the set identifier or the name from some choices. As a result, the client acquires the set identifier according to the input signal from the input 64 and sends it to the server 21 (step S17). Then, the server 21 adds 1 to the value of the non-use-number-of-times information 42 (see FIGS. 27 and 32) related to the set identifier 45 that is the same as the set identifier (set identifier received from the client 61) of the collected set and overwrites the non-use-number-of-times information 42 in the storage 22, with relevance to the set identifier 45 (step S77). It should be noted that, in the client system 6, the client 61 may add 1 to the value of the non-use-number-of-times information 42 (see FIG. 35) related to the set identifier of the collected set and overwrites that non-use-number-of-times information 42 in the storage 62.

In the aforementioned preferred embodiments, the values of the total-number-of-non-uses information 35 and 44 for each given item category are incremented in the server 21 or the client 61 (see steps S73 and step S74) in response to the operator's successive entries of the unique identifiers of the given medical instruments using the input 64 (see step S16), and the number-of-non-uses information 52a for each given item category is stored (see step S76).

On the other hand, the operator may count the number of given medical instruments for each given item category belonging to a given set and enter a counted value for each given item category using the input 64. For example, when the operator selects (e.g., clicks or taps) the item category name display area 94a depending on the number of times equal to the counted value or selects the item category name display area 94a once and then enters a numerical value of the counted value by operating the input 64 in a screen shown in FIG. 39, the entered counted value is displayed in a NUMBER-OF-NON-USES display area 94c on the left of the item category name display area 94a. As a result, the client 61 acquires a counted value for each given item category according to the input signals from the input 64. The client 61 then sends, the acquired counted value to the server 21 with relevance to the item category identifier (which is the same as the item category identifier 28c acquired at step S14) and the set identifier (which is the same as the set identifier acquired at step S12). Thereafter, the server 21 adds the counted value to the value of the total-number-of-non-uses information 35 (see FIGS. 27 and 31) related to the item category identifier 36 (see FIG. 31) that is the same as the item category identifier received from the client 61 and overwrites the total-number-of-non-uses information 35 after the addition in the storage 22. Furthermore, the server 21 adds the counted value to the value of the total-number-of-non-uses information 44 see FIGS. 27 and 32) related to the item category identifier 46 and the set identifier 45 (see FIG. 32) that are identical to the item category identifier and the set identifier received from the client 61 and overwrites the total-number-of-non-uses information 44 after the addition in the storage 22.

Furthermore, the server 21 stores, in the storage 22, the number-of-non-uses information 52a representing the counted value received from the client 61, the count-time-instant information 52b representing the time instant at which the counted value is received from the client 61, the set identifier 52c that is the same as the set identifier received from the client 61, the item category identifier 52d that is the same as the item category identifier received from the client 61, and the user ID 52e of the user who has logged in, with relevance to each other.

In the client system 6, the client 61 may add the counted value (input value from the input 64) to the value of the total-number-of-non-uses information 35 (see FIG. 35) related to the item category identifier 36 that is the same as item category identifier related to the item category name in the item category name display area 94a and overwrite the total-number-of-non-uses information 35 after the addition in the storage 62. Furthermore, the client 61 may add the counted value (input value from the input 64) to the value of the total-number-of-non-uses information 44 (see FIG. 35) related to the item category identifier 46 and the set identifier 45 that are the same as the set identifier and the item category identifier identical to the ones in the display areas 93 and 94a and overwrite the total-number-of-non-uses information 44 in the storage 62. Furthermore, the client 61 may store, in the storage 62, the number-of-non-uses information 52a representing the counted value (input value from the input 64), the count-time-instant information 52b representing the time instant at which the counted value is entered, the set identifier 52c that is the same as the set identifier identical to the one in the set name display area 93, the item category identifier 52d that is the same as the item category identifier identical to the one in the item category name display area 94a, and the user ID 52e of the user who has logged in, with relevance to each other.

With the functions of the management system 1 as described above, the number of times each medical instrument has been transferred and the number of times each medical instrument was not used are tallied up for each medical instrument (see FIG. 30), the total number of transfers and the total number of non-uses are tallied up for each item category (see FIG. 31), the total number of transfers and the total number of non-uses are tallied up for each set and item category (see FIG. 32), and logs of the number of transferred ones and the number of unused ones are tallied up (FIGS. 33 and 34). This management system 1 has a function of calculating a percentage of non-usage using tallied information 31, 32, 34, 35, 41, 42, 43, and 44 as well as data 51 and 52.

The server 21 calculates a percentage of non-usage by dividing the value of the non-use-number-of-times information 32 by the value of the transfer-number-of-times information 31 for each unique identifier 33. The calculated percentage of non-usage is a percentage of non-usage for each medical instrument. Thereafter, the server 21 allows the calculation result for each unique identifier 33 to be displayed on the display 23 with relevance to the unique identifier 33.

The server 21 also calculates a percentage of non-usage by dividing the value of the total-number-of-non-uses information 35 by the value of the total-number-of-transfers information 34 for each item category identifier 36. The calculated percentage of non-usage is a percentage of non-usage for each item category. Thereafter, the server 21 allows the calculation result for each item category identifier 36 to be displayed on the display 23 with relevance to the item category identifier 36 or an item category name (the item category name is obtained as the item category name information 27b by looking up the classification table 27).

The server 21 also calculates a percentage of non-usage by dividing the value of the non-use-number-of-times information 42 by the value of the transfer-number-of-times information 41 for each set identifier 45. The calculated percentage of non-usage is a percentage of non-usage for each set. Thereafter, the server 21 allows the calculation result for each set identifier 45 to be displayed on the display 23 with relevance to the set identifier 45 or a set name (the set name is obtained as the set name information 28b by looking up the set information table 28).

Furthermore, the server 21 calculates a percentage of non-usage by dividing the value of the total-number-of-non-uses information 44 by the value of the total-number-of-transfers information 43 for each set identifier 45 and item category identifier 46. The calculated percentage of non-usage is a percentage of non-usage of the item category for each set and each item category. Thereafter, the server 21 allows the calculation result for each set identifier 45 and item category identifier 46 to be displayed on the display 23 with relevance to the item category identifier 46 or an item category name (the item category name is obtained as the item category name information 28d by looking up the set information table 28) and the set identifier 45 or a set name (the set name is obtained as the set name information 28b by looking up the set information table 28).

Furthermore, the server 21 reads the number-of-transfers log data 51 and sums up the values of the number-of-transfers information 51a related to the same item category identifier 51d. The server 21 also reads the number-of-non-uses log data 52 and sums of the values of the number-of-non-uses information 52a related to the same item category identifier 52d. The server 21 then calculates a percentage of non-usage by dividing the sum of the number-of-non-uses information 52a by the sum of the number-of-transfers information 51a for the same item category identifiers 51d and 52d. The calculated percentage of non-usage is a percentage of non-usage for each item category. Thereafter, the server 21 allows the calculation result for the same item category identifiers 51d and 52d to be displayed on the display 23 with relevance to the item category identifiers 51d and 52d or an item category name (the item category name is obtained as the item category name information 28d by looking up the set information table 28).

The server 21 reads the number-of-transfers log data 51 and sums up the values of the number-of-transfers information 51a related to the same set identifier 51c and the same item category identifier 51d. The server 21 also reads the number-of-non-uses log data 52 and sums up the values of the number-of-non-uses information 52a related to the same set identifier 52c and the same item category identifier 52d. Then, the server 21 calculates a percentage of non-usage for the same set identifiers 51c and 52d as well as the same item category identifiers 51d and 52d by dividing the sum of the number-of-non-uses information 52a by the sum of the number-of-transfers information 51a. The calculated percentage of non-usage is a percentage of non-usage of the item category for each set and each item category. Thereafter, the server 21 allows the calculation result for the same item category identifiers 51d and 52d to be displayed on the display 23 with relevance to the item category identifiers 51d and 52d or an item category name (the item category name is obtained as the item category name information 28d by looking up the set information table 28) and the set identifiers 51c and 52d or a set name (the set name is obtained as the set name information 28b by looking up the set information table 28).

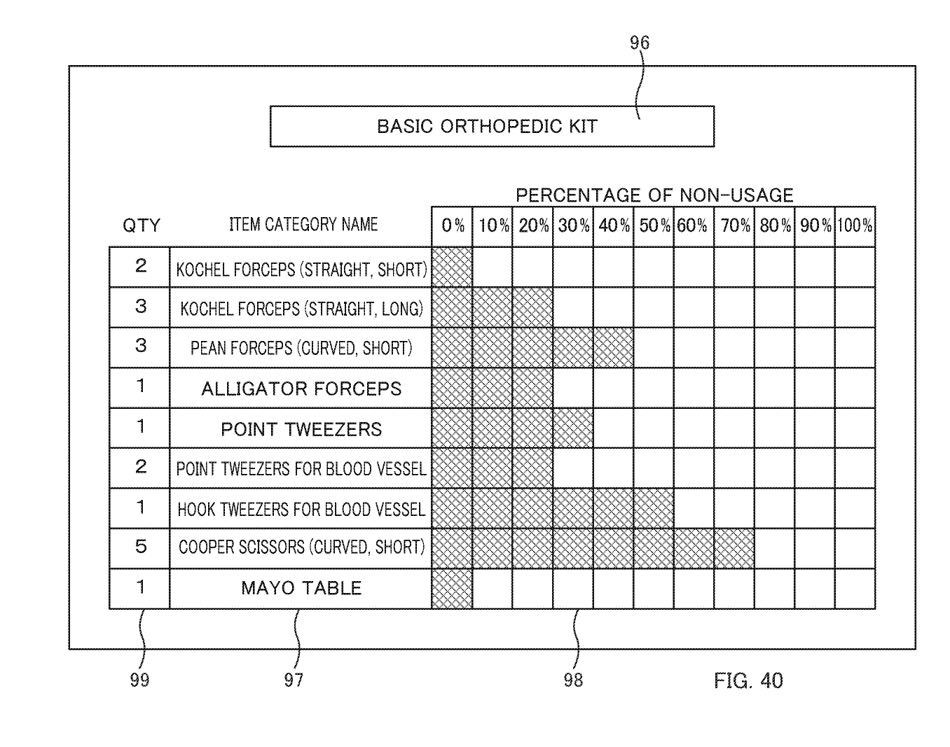

Each calculation result is displayed by, for example, a numerical value (text), symbol, mark, pattern, or indicator. FIG. 40 is an example of a display screen for the calculation result. As shown in FIG. 40, the set name (the set name is obtained as the set name information 28b by looking up the set information table 28) corresponding to the set identifier 45 is displayed in a set name display area 96 on the screen. In addition, item category names (each item category name is obtained as the item category name information 28d by looking up the set information table 28) corresponding to the item category identifier 46 is displayed in an item category name display area 97. A percentage of non-usage (a ratio expressed as a fraction of 100) obtained by dividing the value of the total-number-of-non-uses information 44 by the value of the total-number-of-transfers information 43 is displayed as an indicator in a percentage-of-non-usage display 98 on the right of the item category name display area 97. The number of medical instruments belonging to a set and an item category (the number of them is obtained as the quantity information 28e by looking up the set information table 28) is displayed in a quantity display area 99 on the left of the item category name display area 97.

It should be noted that, with the percentage of non-usage calculated by the server 21, the calculation result may be transmitted to the client 61 and the client 61 may allow the calculation result to be displayed on the display 63. Alternatively, in a manner similar to the one how the server 21 calculates the percentages of non-usage, the client 61 may read the pieces of information 31, 32, 34, 35, 41 to 44 and the data 51 and 52 from the storage 62 and calculate each percentage of non-usage, and the client 61 may allow the calculation result to be displayed on the display 63.

According to the preferred embodiments described above, the following effects are obtained.

Since the classification table 27 is stored in the storage 22, it becomes possible to accurately manage classifications of medical instruments for each item category using the server 21.

Furthermore, since the set information table 28 is stored in the storage 22, it becomes possible to accurately manage grouping of item categories for each set using the server 21. Even when medical instruments belonging to the same item category are interchanged between different sets, the management by the server 21 is not affected.

Accordingly, it is possible to standardize classifications for medical instruments for each item category in a hospital and avoid excessive stock of medical instruments in the entire hospital.

As shown in FIG. 30, since the number of times (sum) each medical instrument was not used is stored in the storage 22 or the storage 62 (see step S75) for each medical instrument, it is possible to easily and accurately manage the number of times each medical instrument was not used.

As shown in FIG. 31, since the total number of unused medical instruments (sum) is stored in the storage 22 or the storage 62 (step S75) for each item category, such total number can be managed easily and accurately.

As shown in FIG. 32, since the number of times (sum) each set was not used is stored in the storage 22 or the storage 62 (step S77) for each set, it is possible to easily and accurately manage the number of times each set was not used.

As shown in FIG. 32, since the total number of unused medical instruments for each set and item category (sum) is stored in the storage 22 and the storage 62 (step S74), it is possible to easily and accurately manage such total number for each set and item category.

Since the number of unused medical instruments for each transferred item category is stored as a log of the number-of-non-uses log data 52 in the storage 22 or the storage 62 with relevance to a time instant and a user ID, it is possible to find when and who counts the number of unused medical instruments.

Although the non-use number of times, the number of unused ones, and the total number of non-uses are counted in the aforementioned preferred embodiments, the number of used times, the number of used ones, and the total number of used ones may be counted. In such cases, the term “unused” should read “used” and the term “used” should read “unused” in the above description of the preferred embodiments except for the definition of the used and unused tools (among the collected tools, a used medical instrument is referred to as a used tool and an unused medical instrument is referred to as an unused tool).

In the aforementioned preferred embodiments, the classification tables 27 and 67 are stored in the storages 22 and 62, respectively, of the management system 1 for managing whether a medical instrument is used or not in a medical procedure. The classification tables 27 and 67 can be applied to any management system as long as it is a management system installed in a hospital (any system for managing medical instruments classified into item categories), and the classification tables 27 and 67 are stored in a storage that can be read by a computer constituting such a management system. The same applies to the set information tables 28 and 68.

In the computer in which the classification tables 27 and 67 can read, the storage is used for management of medical instruments (management to specify that which item category each medical instrument is classified into) in any operations of work cycles (transfer, collection, cleaning, assembly, sterilization, and storage) in hospital facilities where medical instruments are handled.

Here, in work cycles in hospital facilities, medical instruments are collected after a medical procedure (a collection operation), disassembled and cleaned (a cleaning operation), and then assembled (an assembly operation), sterilized (a sterilization operation), and then stored (a storage operation). The stored medical instruments are re-transferred (a transfer operation) and used for medical procedures.

### Fifth Preferred Embodiment

A fifth preferred embodiment of the present invention relates to management systems and non-transitory computer-readable media including programs for managing the number of unused medical instruments for each item category.

When a medical procedure such as a surgery or therapy is performed on a patient, it is common to use two or more medical instruments.

In performing a medical procedure, a method of managing medical instruments using a database to efficiently prepare the medical instruments to be used has been known. For example, JP-A-2002-369787 discloses a storage that stores records of instruments used for medical procedures in a database.

In some cases, not all medical instruments are used even though they were prepared for a medical procedure such as a surgery; therefore, it is necessary to accurately manage which medical instrument was not used, and how many which medical instruments belonging to which item category were not used. This management is typically performed by preparing sheets of paper with entry fields for item category names and the number of unused medical instruments for each item category, writing the number of unused medical instruments for each item category, and then filing these sheets of paper. However, the number of sheets is increased over time and it becomes difficult to tally up.

This preferred embodiment makes it possible to accurately manage the number of unused medical instruments and sums thereof for each item category.

According to this preferred embodiment, a management system including an acquirer that acquires a number of unused medical instruments or a number of used medical instruments for each item category for medical instruments; and a storage that stores the number of unused medical instruments or the number of used medical instruments acquired by the acquirer.

The present preferred embodiment makes it possible to accurately manage the number of unused medical instruments and sums thereof for each item category using a computer-based management system.

In the descriptions of the specification and the drawings that follow, at least the following features are disclosed.

A management system including an acquirer that acquires a number of unused medical instruments or a number of used medical instruments for each item category for medical instruments; and a storage that stores the number of unused medical instruments or the number of used medical instruments acquired by the acquirer is disclosed.

A non-transitory computer-readable medium including a program for causing a computer to function as an acquirer that acquires the number of unused medical instruments or the number of used medical instruments for each item category for medical instruments; and a storage that stores the number of unused medical instruments or the number of used medical instruments acquired by the unused number acquirer is disclosed.

With the management systems and non-transitory computer-readable medium including programs mentioned above, the number of unused or used medical instruments for each item category is stored in a storage. Thus, the number of unused or used medical instruments for each item category can be managed accurately and a sum of the numbers of unused or used medical instruments can be calculated easily.

A management system including an adder that acquires a total number of unused medical instruments or a total number of used medical instruments for each item category for medical instruments by summing up the numbers of unused medical instruments or the numbers of used medical instruments; and a storage that stores the total number of unused medical instruments or the total number of used medical instruments acquired by the adder is disclosed.

A non-transitory computer-readable medium including a program for causing a computer to function as an adder that acquires a number of unused medical instruments or a number of used medical instruments for each item category for medical instruments; and a storage that stores the number of unused medical instruments or the number of used medical instruments acquired by the adder is disclosed.

With the management systems and non-transitory computer readable media including programs mentioned above, a sum of the numbers of unused medical instruments (a total number of unused medical instruments) or a sum of the number of used medical instruments (a total number of used medical instruments) for each item category of medical instruments is stored in a storage; therefore, it is possible to easily and accurately manage the total number of unused or used medical instruments for each item category of medical instruments.

Preferably, the management system further includes total-number-of-transfers adder that acquires a total number of transferred medical instruments by summing up the numbers of transferred medical instruments for each item category for medical instruments; and a total-number-of-transfers storage that stores the total number of transferred medical instruments acquired by the total-number-of-transfers adder.

Preferably, the program causes the computer to function as total-number-of-transfers adder that acquires a total number of transferred medical instruments by summing up the numbers of transferred medical instruments for each item category for medical instruments; and the total-number-of-transfers storage that stores the total number of transferred medical instruments acquired by the total-number-of-transfers adder.

With the management systems and non-transitory computer readable media including programs mentioned above, a sum of the numbers of transferred medical instruments (a total number of transferred medical instruments) for each item category of medical instruments is stored in a storage; therefore, it is possible to easily and accurately manage the total number of transferred medical instruments for each item category of medical instruments.

Preferably, the management system further includes a calculator to calculate a ratio between the total number of transferred medical instruments that have been stored by the total-number-of-transfers storage and the total number of unused medical instruments or the total number of used medical instruments that have been stored by storage.

Preferably, the program causes the computer to function as a calculator that calculates a ratio between the total number of transferred medical instruments that have been stored by the total-number-of-transfers storage and the total number of unused medical instruments or the total number of used medical instruments that have been stored by storage.

With the management systems and non-transitory computer readable media including programs mentioned above, it is possible to easily and accurately manage a percentage of non-usage or a percentage of usage.

Referring to the drawings, this preferred embodiment is described below. The preferred embodiment described below includes various features that are technically preferable for the purpose of implementing the present invention; therefore, the scope of the present invention is not limited to the following preferred embodiment and illustrated examples.

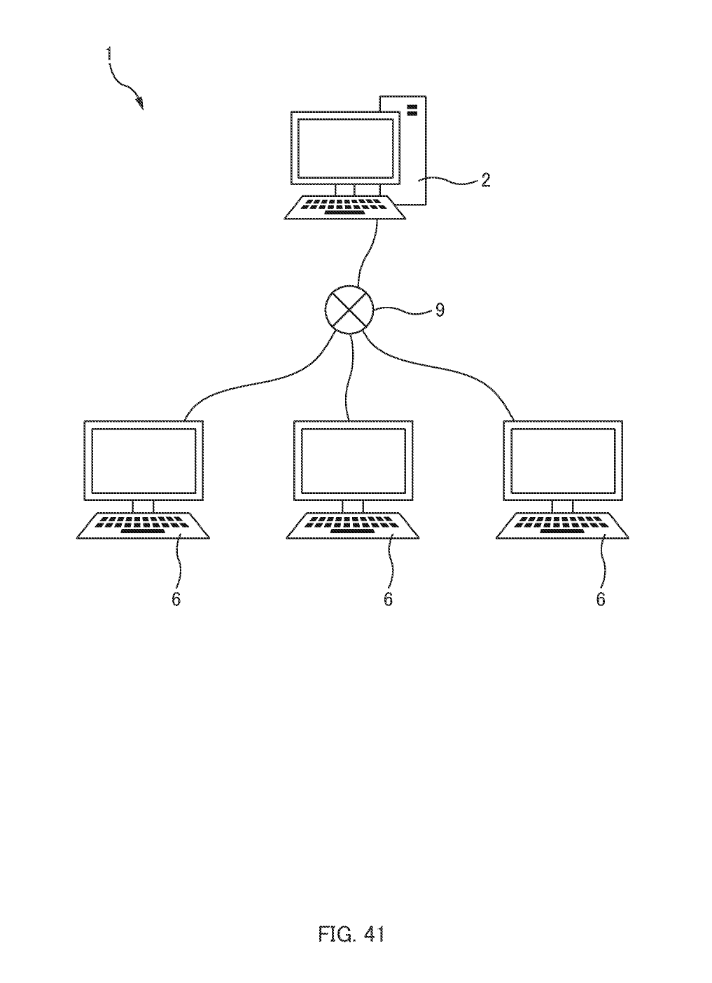

As shown in FIG. 41, a management system 1 includes a server system 2, a plurality of client systems 6, and a communication network 9. The client systems 6 can communicate with the server system 2 via the network 9.

Each client system 6 is a desktop type, laptop type or tablet type computer system. Each client system 6 may be a portable computer system or a computer system on a desk.

The server system 2 is a tower type, desktop type, rack mount type or blade type computer system. The communication network 9 is an intranet, a local area network (LAN), the Internet, or the like.

The management system 1 is a system for managing whether medical instruments that have been transferred (delivered) from a storage place to an operating place are used in a medical procedure. Specifically, the management system 1 is a system for summing up the number of times that the medical instruments were not used even though they had been transferred from the storage place to the operating place.

An operating place in a hospital refers to a place where surgery, medication, treatment, examination or other medical procedure is performed; for example, a treatment room, an operating room, a medical room or an examination room. The client systems 6 are provided at the operating place(s). The server system 2 can be installed in a hospital facility where the operating place is located or installed in a remote location such as a server provider and the like.

The medical instruments refer to instruments such as endoscopes, ultrasonic probes, pairs of forceps, pairs of scissors, scalpels, scalpel handles, cannulas, tweezers, retractors, scales, Sondes, elevators, raspas, suction tubes, rib spreaders, rib contractors, needle holders, syringes, metal balls, kidney dishes, cups, pins, mirrors, files, opening devices, Klemmes, handpieces, Elevatoriums, chisels, curettes, raspatories, mirrors, suture needles, rongeurs, water receivers, needles, spatulas, bougies, vent tubes, bone impactors, rongeurs, needle-nose pliers, hammers, goniometers, perforators, droppers, metal swabs, enemas, syringes and the like.

Each medical instrument is given a unique identifier. For example, medical instruments may be provided with a two-dimensional code, a one-dimensional code, a mark, a numerical value, a pattern or an image that represents a unique identifier or an RF tag storing information representing a unique identifier.

Medical instruments are classified into item categories, but a unique identifier for each item category is not attached to the medical instruments.

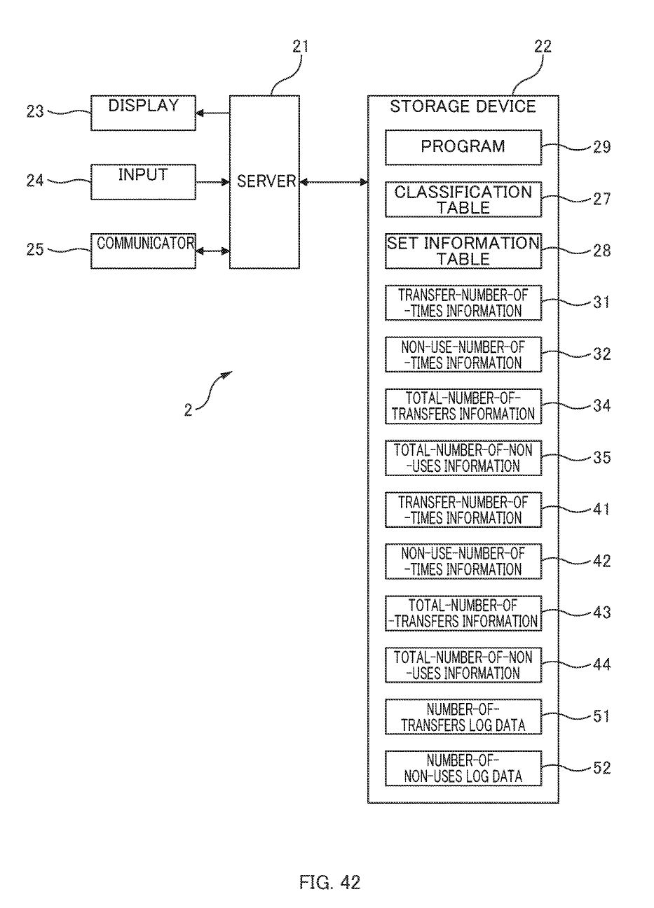

Any range can be defined for each item category. For example, medical instruments from different manufacturers (their applications and functions are the same) may be regarded as the same item category. In addition, the medical instruments for the same purpose having the same function may be regarded as a different item category when they are different in size, shape or color etc. Furthermore, the medical instruments belonging to the same item category may have different names. The item category classifications for medical instruments are defined by using classification tables 27 and 67 (see FIGS. 42, 43, and 50) described later.

Medical instruments to be transferred from a storage place to an operating place are divided into groups, and a collection of medical instruments constituting a group is called a set. That is, two or more medical instruments belong to the set. The medical instruments belonging to the set are delivered the operating place in a state of being gathered, for example, in a container (for example, a tray, a storage basket, a storage box, a storage bag or a storage container) or assembled.

Each set is given a unique set identifier. For example, a set may be provided with a two-dimensional code, a one-dimensional code, a mark, a numerical value, a pattern or an image that represents a set identifier or an RF tag storing data representing a unique identifier. Assigning a set identifier to a set means that a set identifier is attached to some or all of the medical instruments belonging to the set, that a set identifier is attached to a container in which the medical instruments belonging to the set are gathered, and that a set identifier is attached to a set of medical instruments belonging to the set, and the like.

In the storage place, the medical instruments may be stored after being divided as separate sets or may be stored after being divided into separate item categories. In the latter case, the operator picks out the medical instruments and puts them together before releasing them to an operating place.

Regardless of whether used or not, the medical instruments delivered to an operating place are returned to the storage place after being collected, cleaned and sterilized after the medical procedure. Thus, the medical instruments are reused.

As shown in FIG. 42, this server system 2 preferably includes a server 21, a storage 22, the display 23, an input 24, and a communicator 25.

The server 21 is a computer having a CPU, a GPU, a ROM, a RAM, a bus, a hardware interface, and the like. The storage 22 may be a semiconductor memory, a hard disk drive, and the like that is readable and writable by the server 21. The display 23 is a display that performs screen display. The input 24 is an input interface such as a touch panel, a switch, a keyboard, a pointing device, an RFID reader, a one-dimensional code reader, and a two-dimensional code reader. The communicator 25 is a network adapter (for example, a network interface card, a wireless LAN slave).

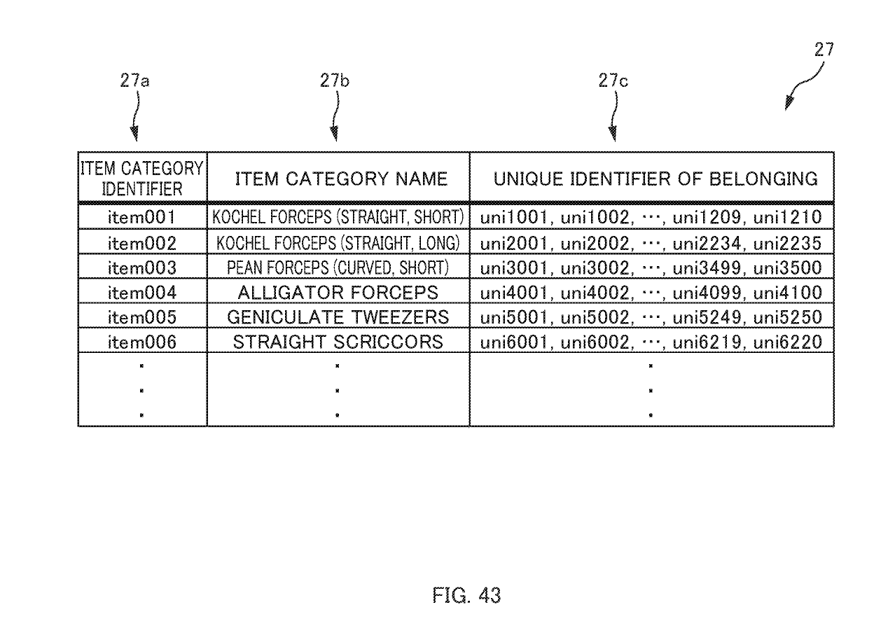

The storage 22 has stored thereon a classification table 27 to classify medical instruments into item categories. FIG. 43 is a diagram showing an example of a configuration of the classification table 27. The classification table 27 includes item category identifiers 27a each of which is unique to an item category, pieces of item category name information 27b which are related to the item category identifiers 27a and each of which represents an item category name, and unique identifiers 27c which are related to the item category identifiers 27a for the medical instruments that belong to the item categories. Each item category identifier 27a is a common ID that is shared by all of the unique identifiers 27c related thereto. It means that medical instruments associated with a unique identifier that is the same as the unique identifier 27c belong to the item category of the item category identifier 27a related to the unique identifier 27c. It should be noted that, since the same medical instruments do not belong to different item categories, the same unique identifiers 27c are not related to different item category identifiers 27a.

When the server 21 refers to the classification table 27 as mentioned above, any item category identifier 27a can be specified from the unique identifier 27c and any unique identifier 27c can be specified from the item category identifier 27a. Accordingly, by using the server 21, it becomes possible to accurately manage classifications of instruments for each item category.

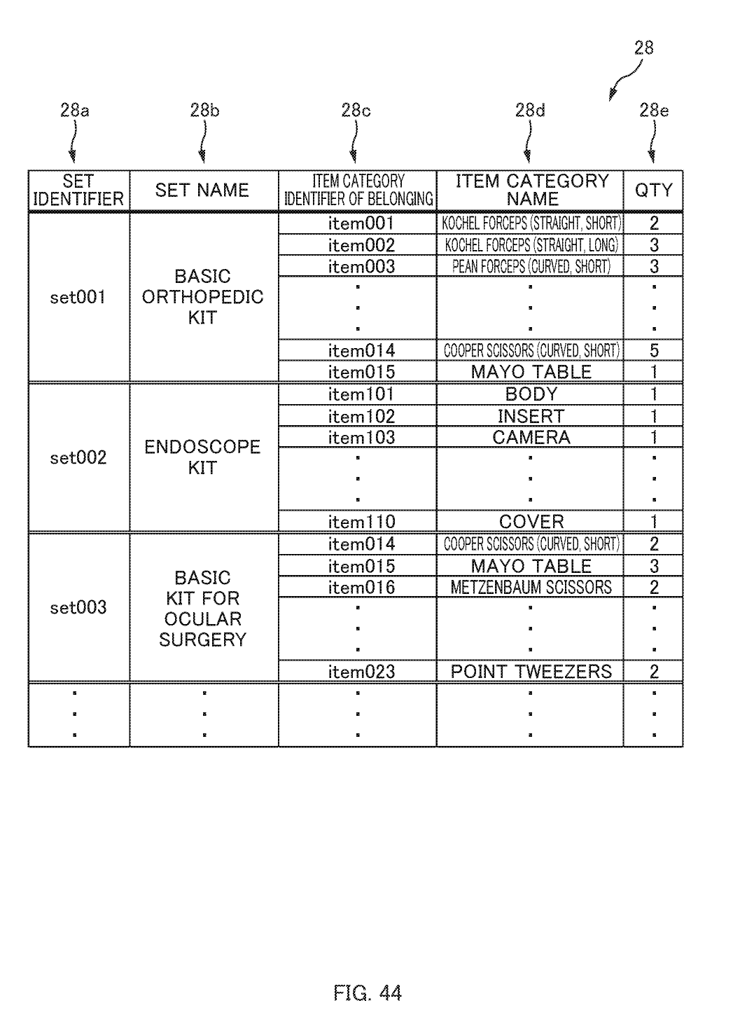

Furthermore, the storage 22 has stored a set information table 28 that represents combinations of item categories that belong to sets. FIG. 44 a diagram showing an example of a configuration of the set information table 28. The set information table 28 includes set identifiers 28a unique to each set, pieces of set name information 28b which are related to the set identifiers 28a and each of which represents a set name, item category identifiers 28c which are related to the set identifier 28a for the item categories that belong to the sets, pieces of item category name information 28d which are related to the set identifiers 28a and the item category identifiers 28c and each of which represents an item category name, and pieces of quantity information 28e which are related to the set identifiers 28a and the item category identifiers 28c and each of which represents the number of medical instruments that belong to the sets and the item categories. It should be noted that, since the same item category may belong to different sets, the same item category identifier 28c may be related to different set identifiers 28a.

When the server 21 looks up the set information table 28 as described above, it is possible to specify the set identifier 28a from the item category identifier 28c or specify the item category identifier 28c from the set identifier 28a. Thus, it becomes possible to manage grouping of item categories for each set by using the server 21. Furthermore, the item category identifiers 28c are related to the set identifiers 28a in the set information table 28, and the unique identifiers 27c are related to the item category identifiers 27a in the classification table 27. Accordingly, even when the medical instruments that belong to the same item category are interchanged between different sets, the server 21 can recognize that the medical instruments after the exchange also belong to the same item category by looking up the classification table 27 and the set information table 28.

The storage 22 has stored thereon a program 29 that can be executed by the server 21.

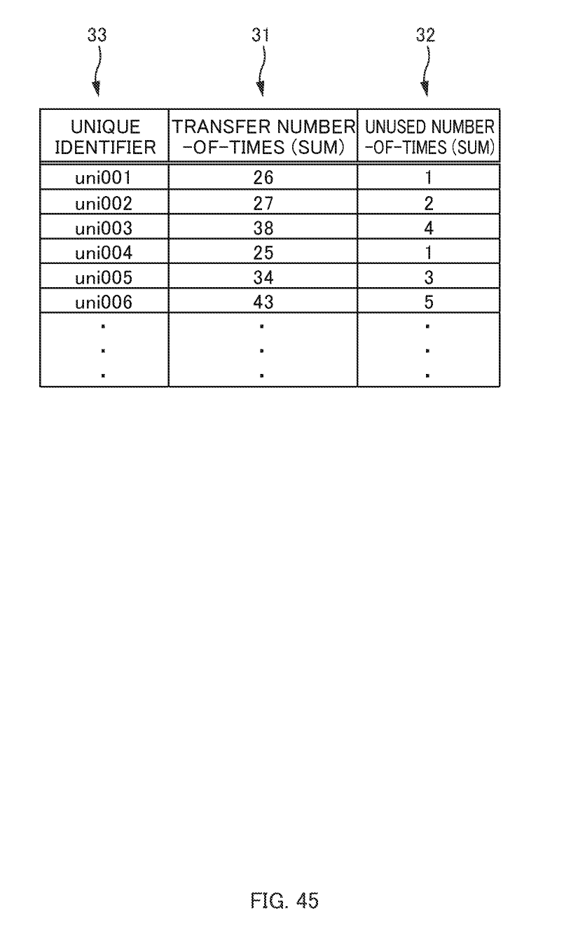

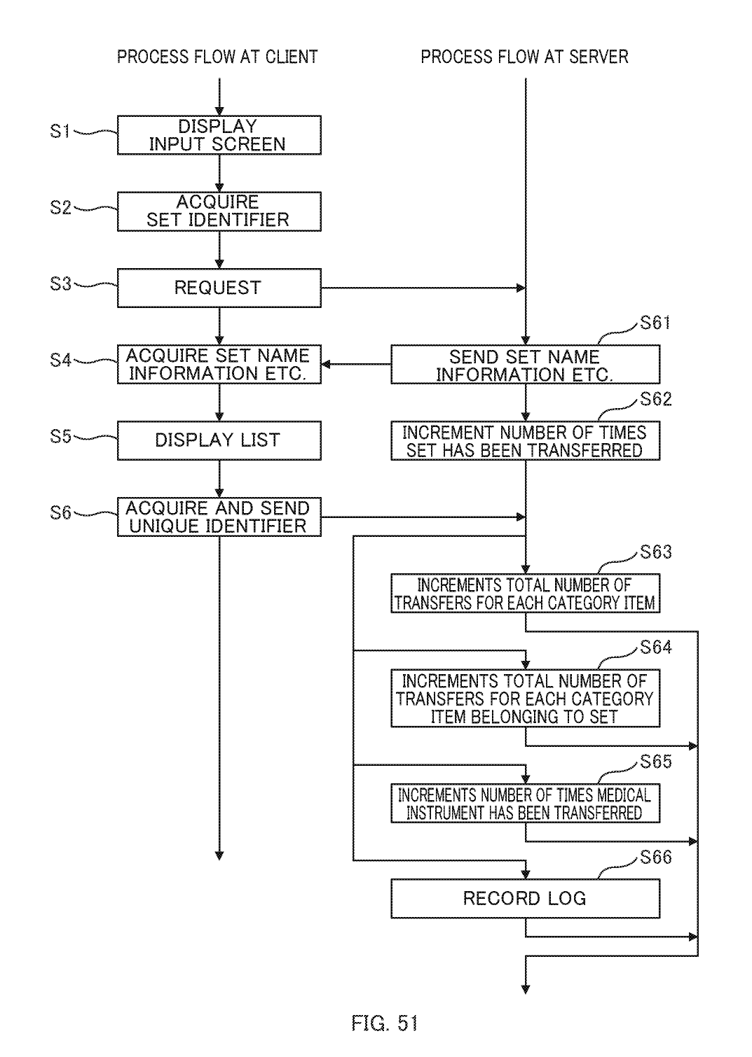

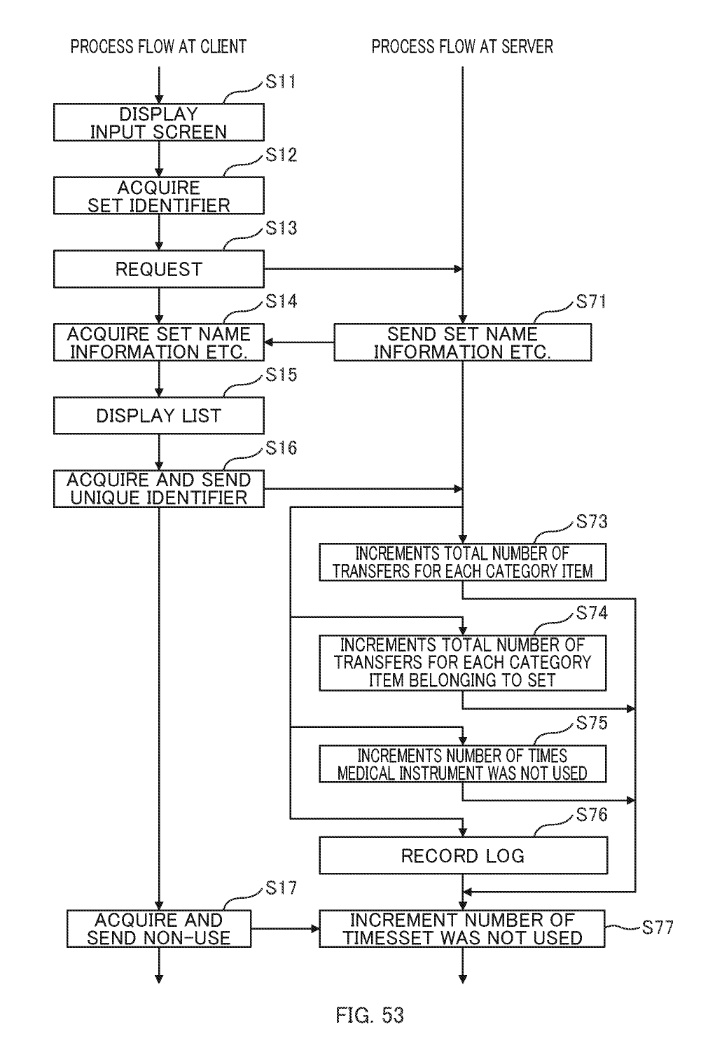

In the storage 22, transfer-number-of-times information 31 that represents the number of times each medical instrument has been transferred is stored and updated by an operation performed at step S65 described below (see FIG. 51). In the storage 22, non-use-number-of-times information 32 that represents the number of times each medical instrument was not used (the number of times it remained unused even though it had been transferred) is stored and updated by an operation performed at step S75 described below (see FIG. 53). Each of the transfer-number-of-times information 31 and the non-use-number-of-times information 32 is a current sum and the initial value for each of the transfer-number-of-times information 31 and the non-use-number-of-times information 32 is zero. As shown in FIG. 45, the transfer-number-of-times information 31 and the non-use-number-of-times information 32 are stored and updated in the storage 22 with relevance to a unique identifier 33 for each medical instrument.

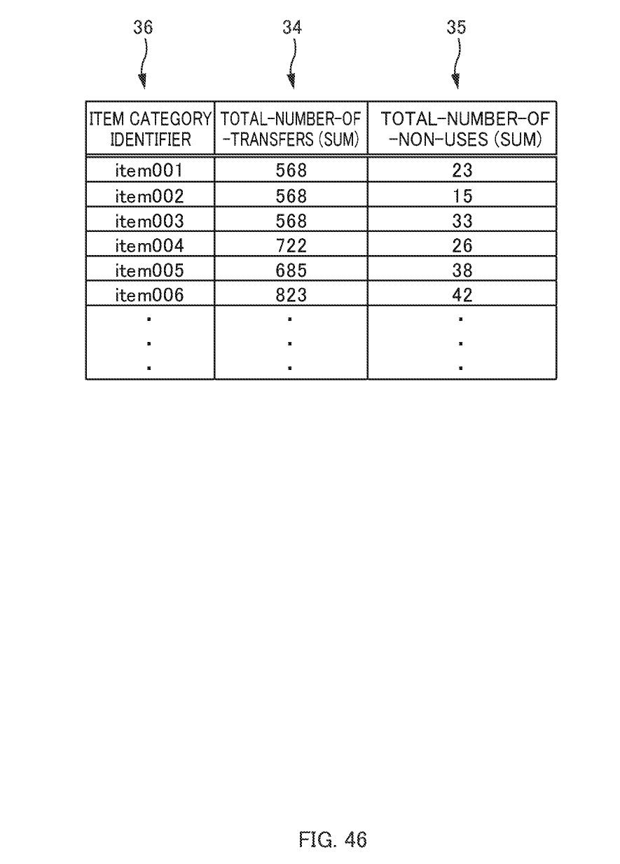

In the storage 22, total-number-of-transfers information 34 that represents the total number of times medical instruments for each item category has been transferred (total number of transferred medical instruments that belong to each item category) is stored and updated by an operation performed at step S63 described below (see FIG. 51). In the storage 22, total-number-of-non-uses information 35 that represents the total number of medical instruments that were not used for each item category (the total number of medical instruments that belong to item categories remained unused even though they had been transferred) is stored and updated by an operation performed at step S73 described below (see FIG. 53). Each of the total-number-of-transfers information 34 and the total-number-of-non-uses information 35 is a current sum and the initial value for each of the total-number-of-transfers information 34 and the total-number-of-non-uses information 35 is zero. As shown in FIG. 46, the total-number-of-transfers information 34 and the total-number-of-non-uses information 35 are stored and updated in the storage 22 with relevance to an item category identifier 36 that is unique to each item category.

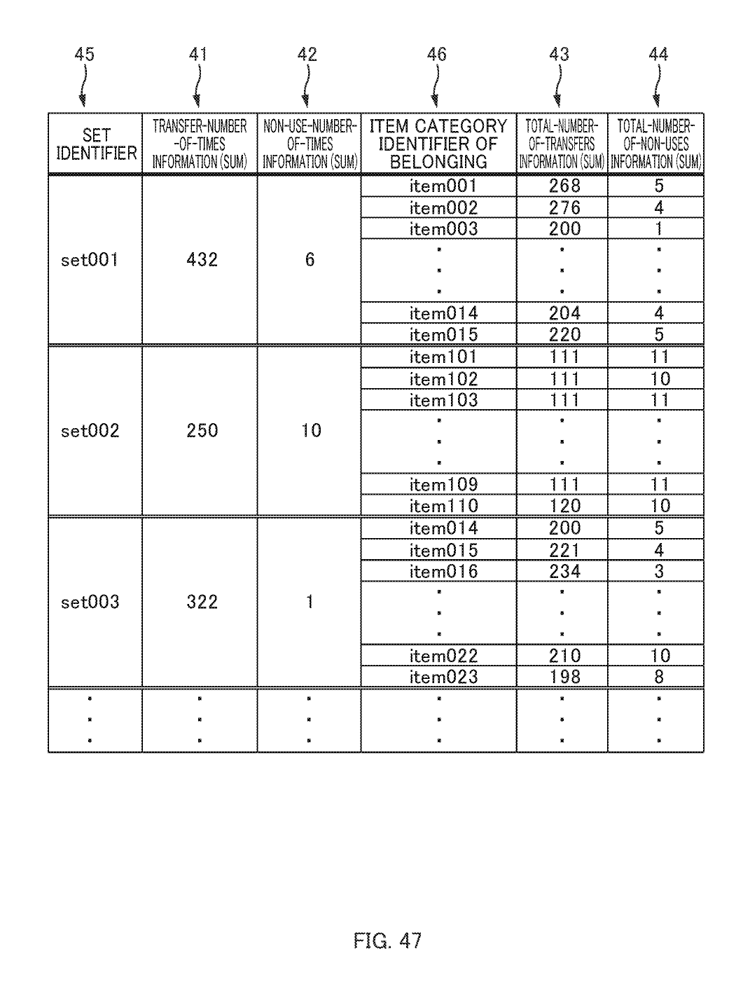

In the storage 22, transfer-number-of-times information 41 that represents the number of times each set has been transferred is stored and updated by an operation performed at step S62 described below (see FIG. 51). In the storage 22, non-use-number-of-times information 42 that represents the number of times each set was not used (the number of times it remained unused even though it had been transferred) is stored and updated by an operation performed at step S76 described below (see FIG. 53). Each of the transfer-number-of-times information 41 and the non-use-number-of-times information 42 is a current sum and the initial value for each of the transfer-number-of-times information 41 and the non-use-number-of-times information 42 is zero. As shown in FIG. 47, the transfer-number-of-times information 41 and the non-use-number-of-times information 42 are stored and updated in the storage 22 with relevance to a set identifier 45 for each set.

In the storage 22, total-number-of-transfers information 43 that represents the total number of transfers for each item category that belongs to a set is stored and updated by an operation performed at step S64 described below (see FIG. 51). In the storage 22, total-number-of-non-uses information 44 that represents the total number of medical instruments that were not used for each item category that belongs to a set (the total number of medical instruments that belong to a set and an item category remained unused even though they had been transferred) is stored and updated by an operation performed at step S74 described below (see FIG. 53). Each of the total-number-of-transfers information 43 and the total-number-of-non-uses information 44 is a current sum and the initial value for the total-number-of-non-uses information 44 is zero. As shown in FIG. 47, the total-number-of-transfers information 43 and the total-number-of-non-uses information 44 are stored and updated in the storage 22 with relevance to a set identifier 45 and an item category identifier 46.

The value of the total-number-of-transfers information 43 is the total number (sum) of the medical instruments belonging to an item category of the item category identifier 46 which have been transferred as a set with the set identifier 45. In contrast, the value of the total-number-of-transfers information 34 is the total number (sum) of the medical instruments belonging to an item category of the item category identifier 36 among all of the transferred sets have been transferred. Accordingly, the sum of the values of the total-number-of-transfers information 43 related to the same item category identifier 46 is equal to the sum of the values of the total-number-of-transfers information 34 related to the item category identifier 36 that is the same as the item category identifier 46. The same applies to the total-number-of-non-uses information 35 and the total-number-of-non-uses information 44. The sum of the values of the total-number-of-non-uses information 44 related to the same item category identifier 46 is equal to the sum of the values of the total-number-of-non-uses information 35 related to the item category identifier 36 that is the same as the item category identifier 46.

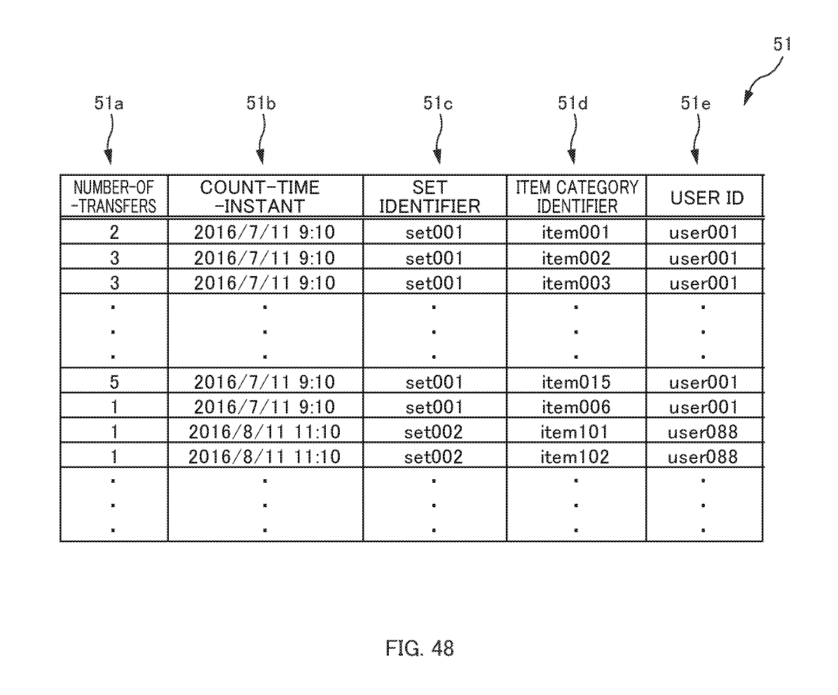

In the storage 22, a number-of-transfers log data 51 is stored by an operation performed at step S66 described below (see FIG. 51). As shown in FIG. 48, the number-of-transfers log data 51 is a relational representation of number-of-transfers information 51a that represents the number of medical instruments that have been transferred for each item category per a single transfer of a set, count-time-instant information 51b that represents the time instant at which the number of the transferred medical instruments was counted, one or more set identifiers 51c, one or more item category identifiers 51d, and one or more user IDs 51e. It should be noted that the time is in units of days, hours, minutes or seconds, or less than seconds.

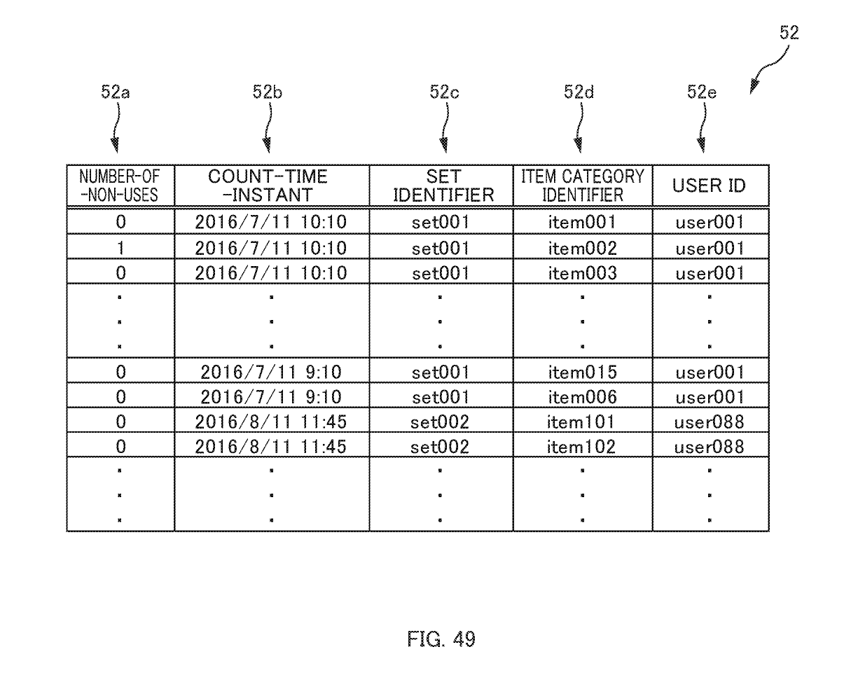

In the storage 22, number-of-non-uses log data 52 is stored by an operation performed at step S76 described below (see FIG. 53). As shown in FIG. 49, the number-of-non-uses log data 52 is a relational representation of number-of-non-uses information 52a that represents the number of unused medical instruments for each item category per a single transfer of a set, count-time-instant information 52b that represents the time instant at which the number of the unused medical instruments was counted, one or more set identifiers 52c, one or more item category identifiers 52d, and one or more user IDs 52e. It should be noted that the time is in units of days, hours, minutes or seconds, or less than seconds.

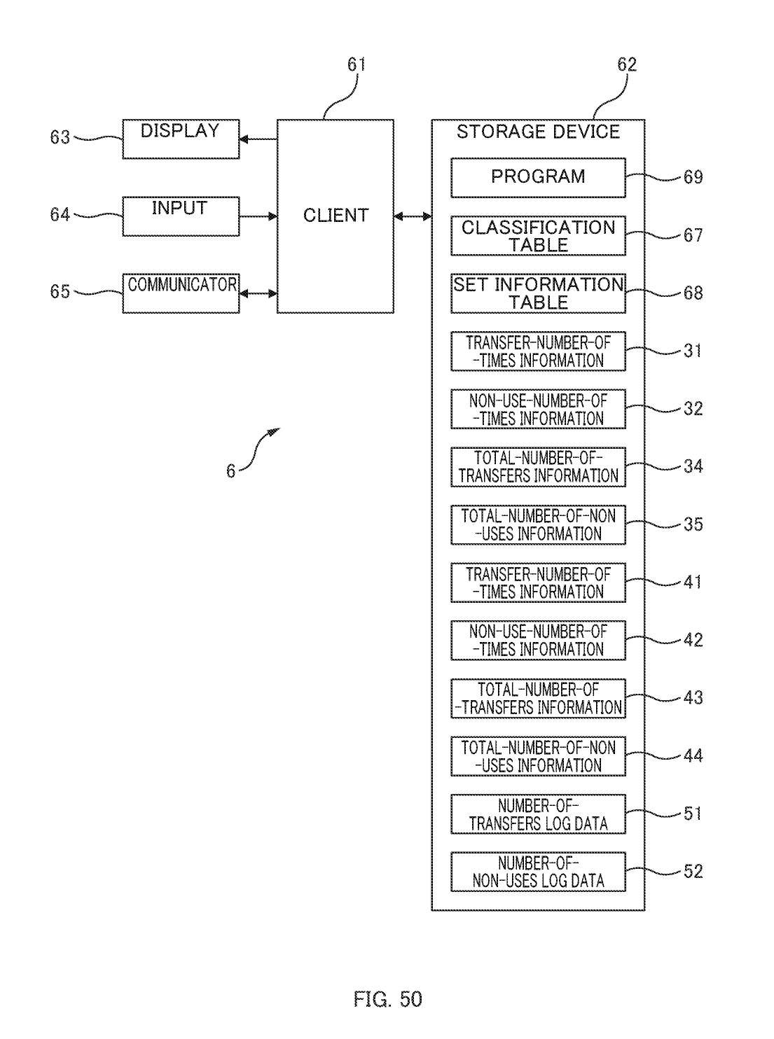

These pieces of information 31, 32, 34, 35, 41 to 44 and data 51 and 52 may be stored and updated in a storage 62 of a client system 6 (see FIG. 50) described below.

As shown in FIG. 50, a client system 6 preferably includes a client 61, a storage 62, a display 63, an input 64, and a communicator 65.

The client 61 is a computer preferably including a CPU, a GPU, a ROM, a RAM, a bus, a hardware interface, and the like. The storage 62 may be a semiconductor memory, a hard disk drive, and the like that is readable and writable by the client 61. The display 63 is a display that performs screen display. The input 64 is an input interface such as a touch panel, a switch, a keyboard, a pointing device, an RFID reader, a one-dimensional code reader, and a two-dimensional code reader. The communicator 65 is a network adapter (for example, a network interface card, a wireless LAN slave).

The storage 62 has stored thereon a program 69 that can be executed by the client 61.

Furthermore, in the storage 62, a classification table 67 and a set information table 68 are stored. The classification table 67 is in synchronism with the classification table 27 stored in the storage 22 of the server system 2 and the set information table 68 is in synchronism with the set information table 28 stored in the storage 22. Accordingly, the classification table 67 includes the item category identifier 27a, the item category name information 27b, and the unique identifier 27c which are related to each other, as in the case of the classification table 27 shown in FIG. 43. Furthermore, the set information table 68 includes the set identifier 28a, the set name information 28b, the item category identifier 28c, the item category name information 28d, and the quantity information 28e which are related to each other, as in the case of the set information table 28 shown in FIG. 44. Here, by the client 61 requesting to the server 21, the classification table 27 (or a difference between the classification table 67 and the classification table 27) is transferred from the server 21 to the client 61. This synchronizes the classification table 67 of the storage 62 and the classification table 27 of the storage 22. The same applies to the set information tables 28 and 68.

The function of this synchronization between the server 21 and the client 61 is achieved by the programs 29 and 69.

A method of using the management system 1 at the time of transferring medical instruments will be described. Besides, referring to FIG. 51, a flow of processing that the program 29 causes the server 21 to execute and a flow of processing that the program 69 causes the client 61 to execute in using the management system 1 are also described.

First, an operator releases a set from a storage place to an operating place. In the following description, a set transferred to an operating place is referred to as a given set, a medical instrument belonging to the given set is referred to as a given instrument, and an item category belonging to the given set is referred to as a given item category.

Next, the operator operates the input 64 of the client system 6 at the operating place and logs in to the management system 1 using his own user ID and password. Then, the client 61 causes the display 63 to display a set identifier input screen (set selection screen) (step S1).

Thereafter, the operator enters a set identifier for the given set via the input 64, and selects a name or a set identifier of the given set from several options. As a result, the client 61 gets the set identifier according to the input signal from the input 64 (step S2). As described above, the input 64 may be a touch panel, a switch, a keyboard, a pointing device, an RFID reader, a one-dimensional code reader, and a two-dimensional code reader, or the like, and the set identifier is entered or selected by the operation depending on the kind of the input 64.

Next, the client 61 requests the server 21 to send information of the given set by sending the set identifier of the given set to the server 21 (step S3). Then, the server 21 looks up the set information table 28, retrieves the set name information 28b, the item category identifier 28c, the item category name information 28d, and the quantity information 28e which are related to the set identifier 28a that is the same as the set identifier of the given set, and sends them to the client 61 (step S61). On the other hand, the client 61 receives and acquires the set name information 28b, the item category identifier 28c, the item category name information 28d, and the quantity information 28e (step S4). It should be noted that the client 61 may look up the set information table 68 in the storage 62 without making a request as step S3 because the set information table 68 that is the same as the set information table 28 is synchronized with the storage 62. That is, the client 61 may retrieve the set name information 28b, the item category identifier 28c, the item category name information 28d, and the quantity information 28e which are related to the set identifier 28a that is the same as the set identifier of the given set, from the set information table 68 in the storage 62.

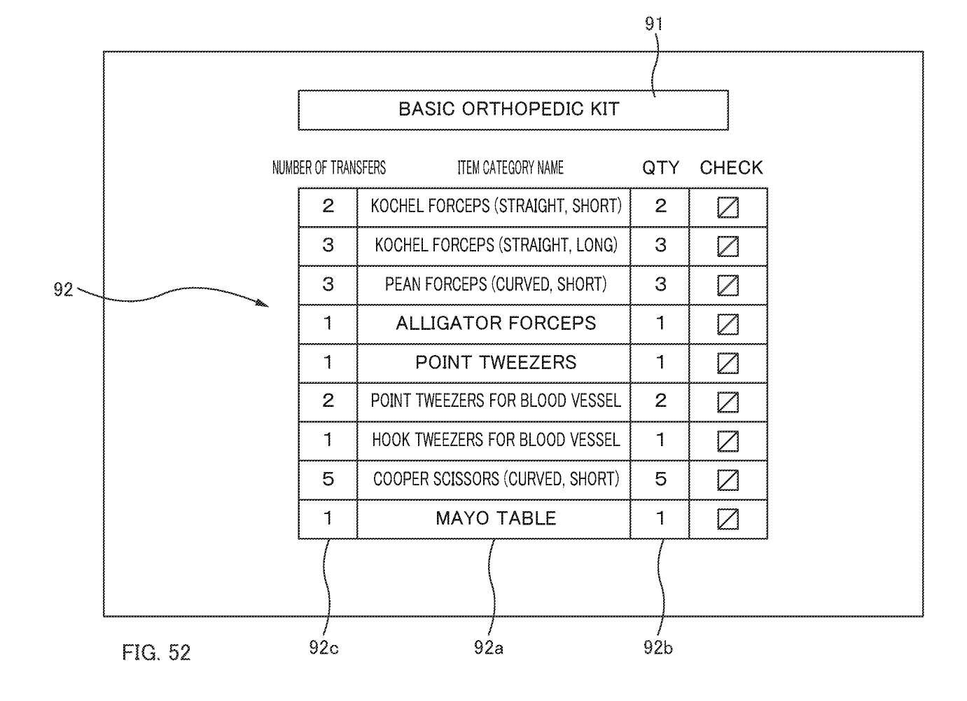

Next, the client 61 allows a screen as shown in FIG. 52 to be displayed on the display 63 according to the set name information 28b, the item category identifier 28c, the item category name information 28d, and the quantity information 28e which have been acquired (step S5). In a set name display area 91 on the screen, a set name according to the set name information 28b is displayed. In a list display area 92 on the screen, item category names according to the item category name information 28d and the quantities according to the quantity information 28e are displayed with relevance to each other. Specifically, item category names are displayed in an item category name display area 92a of the list display area 92 and the quantities are displayed in a quantity display area 92b on the right of the item category name display area 92a. Accordingly, the operator can check the contents of the transferred given set (such as the number of the medical instruments for each item category) while looking at the screen.

On the other hand, in the server system 2, the server 21 overwrites the transfer-number-of-times information 41 in the storage 22 by adding 1 to a value of the transfer-number-of-times information 41 related to the set identifier 45 that is the same as the set identifier of the given set (the set identifier received from the client 61) (see FIGS. 42 and 47) and relating that transfer-number-of-times information 41 to the set identifier 45 (step S62). Accordingly, the number of times the given set has been transferred is counted up and the value of the updated transfer-number-of-times information 41 is a sum. It should be noted that the operation at step S62 may be performed by the client 61. That is, after step S2, the client 61 may add 1 to the value of the transfer-number-of-times information 41 in the storage 62 related to the set identifier of the given set (see FIG. 50) and overwrite the transfer-number-of-times information 41 in the storage 62.

Then, the operator enters unique identifiers of given medical instruments one by one using the input 64. In response to this, the client 61 acquires unique identifiers according to the input signals from the input 64 and transmits the acquired unique identifiers to the server 21 with relevance to the set identifier of the given set (step S6).

The server 21 adds 1 to the value of the total-number-of-transfers information 34 related to one of the item category identifiers 36 (see FIGS. 42 and 46) every time when it receives a unique identifier from the client 61 (step S63). Specifically, every time when a unique identifier is received, the server 21 looks up the classification table 27 to specify the item category identifier 27a related to the unique identifier 27c that is the same as the received unique identifier and adds 1 to the value of the total-number-of-transfers information 34 related to the item category identifier 36 that is the same as that item category identifier 27a (step S63). Such operations are repeated until the last unique identifier is received. After the increment in response to the reception of the last unique identifier, the summed-up total-number-of-transfers information 34 is overwritten in the storage 22 by the server 21. Accordingly, the total number of transferred given medical instruments is counted up for each given item category, and the value of the updated total-number-of-transfers information 34 is a sum. It should be noted that the operation at step S63 may be performed by the client 61. That is, until the last unique identifier is acquired (see step S6), every time when a unique identifier is acquired, the client 61 may look up the classification table 67 to specify the item category identifier 27a related to the unique identifier 27c that is the same as the acquired unique identifier and add 1 to the value of the total-number-of-transfers information 34 in the storage 62 related to the item category identifier 36 that is the same as that item category identifier 27a (see FIG. 50).

Furthermore, the server 21 adds 1 to the value of the total-number-of-transfers information 43 related to one of the set identifiers 45 and the item category identifiers 46 (see FIGS. 42 and 47) every time when it receives a unique identifier from the client 61 (step S64). Specifically, every time when a unique identifier is received, the server 21 looks up the classification table 27 to specify the item category identifier 27a related to the unique identifier 27c that is the same as the received unique identifier and adds 1 to the value of the total-number-of-transfers information 43 related to the item category identifier 46 that is the same as that item category identifier 27a and the set identifier 45 that is the same as the received set identifier of the given set (step S64). Such operations are repeated until the last unique identifier is received. After the increment in response to the reception of the last unique identifier, the summed-up total-number-of-transfers information 43 is overwritten in the storage 22 by the server 21. Accordingly, the total number of transferred given medical instruments is counted up for each given item category, and the value of the updated total-number-of-transfers information 43 is a sum. It should be noted that the operation at step S64 may be performed by the client 61. That is, until the last unique identifier is acquired (see step S6), every time when a unique identifier is acquired, the client 61 may look up the classification table 67 to specify the item category identifier 27a related to the unique identifier 27c that is the same as the acquired unique identifier and add 1 to the value of the total-number-of-transfers information 43 related to the item category identifier 46 that is the same as that item category identifier 27a and the set identifier 45 that is the same as the set identifier of the given set (see FIG. 50).

Furthermore, the server 21 adds 1 to the value of the transfer-number-of-times information 31 related to the unique identifier 33 that is the same as the unique identifier received from the client 61 (see FIGS. 42 and 45) and overwrites the transfer-number-of-times information 31 in the storage 22 (step S65). Accordingly, the number of times given medical instruments belonging to the given set have been transferred is counted up and the value of the updated transfer-number-of-times information 31 is a sum. It should be noted that the operation at step S65 may be performed by the client 61. That is, after the acquisition of the unique identifier (step S6), the client 61 may add 1 to the value of the transfer-number-of-times information 31 related to the unique identifier 33 that is the same as the acquired unique identifier and overwrite the transfer-number-of-times information 31 in the storage 62.

As described above, every time when a unique identifier is received, the item category identifier 27a related to the unique identifier 27c that is the same as the received unique identifier is specified by looking up the classification table 27. The server 21 calculates the number of times it specifies the item category identifiers 27a from the reception of the first unique identifier to the reception of the last unique identifier, for each item category identifier 27a. Such a calculated value for each item category identifier 27a is equal to the number of the transferred medical instruments for each item category. The server 21 stores, in the storage 22, the number-of-transfers information 51a representing that calculated value, the count-time-instant information 51b representing the time instant at which the last unique identifier was received, the set identifier 51c that is the same as the set identifier received from the client 61, the item category identifier 51d that is the same as the specified item category identifier 27a, and the user ID 51e of the user who has logged in, with relevance to each other as shown in FIG. 48 (step S66). Here, the number-of-transfers information 51a etc. stored at step S66 is a log for a single processing shown in FIG. 51. The number-of-transfers information 51a etc. are accumulated by repeating the processing shown in FIG. 51, which are the number-of-transfers log data 51. It should be noted that the operation at step S66 may be performed by the client 61. That is, the client 61 may calculate the number of times the item category identifiers 27a are specified from the acquisition of the first unique identifier to the acquisition of the last unique identifier for each item category identifier 27a. In such a case, the client 61 stores, in the storage 62, the number-of-transfers information 51a, the count-time-instant information 51b, the set identifier 51c, the item category identifier 51d, and the user ID 51e, with relevance to each other as the number-of-transfers log data 51.

In the aforementioned preferred embodiments, the values of the total-number-of-transfers information 34 and 43 for each given item category are incremented in the server 21 or the client 61 (see steps S63 and step S64) in response to the operator's successive entries of the unique identifiers of the given medical instruments using the input 64 (see step S6), and the number-of-transfers information 51a for each given item category is stored (see step S66).

On the other hand, the operator may count the number of given medical instruments for each given item category belonging to a given set and enter a counted value for each given item category using the input 64. For example, when the operator selects (e.g., clicks or taps) the item category name display area 92a depending on the number of times equal to the counted value or selects the item category name display area 92a once and then enters a numerical value of the counted value by operating the input 64 in a screen shown in FIG. 52, the entered counted value is displayed in a transferred-quantity display area 92c on the left of the item category name display area 92a. As a result, the client 61 acquires a counted value for each given item category according to the input signals from the input 64. The client 61 then sends, the acquired counted value to the server 21 with relevance to the item category identifier (which is the same as the item category identifier 28c acquired at step S4) and the set identifier (which is the same as the set identifier acquired at step S2). Thereafter, the server 21 adds the counted value to the value of the total-number-of-transfers information 34 (see FIGS. 42 and 46) related to the item category identifier 36 (see FIG. 46) that is the same as the item category identifier received from the client 61 and overwrites the total-number-of-transfers information 34 after the addition in the storage 22. Furthermore, the server 21 adds the counted value to the value of the total-number-of-transfers information 43 see FIGS. 42 and 47) related to the item category identifier 46 and the set identifier 45 (see FIG. 47) that are identical to the item category identifier and the set identifier received from the client 61 and overwrites the total-number-of-transfers information 43 after the addition in the storage 22.

Furthermore, the server 21 stores, in the storage 22, the number-of-transfers information 51a representing the counted value received from the client 61, the count-time-instant information 51b representing the time instant at which the counted value is received from the client 61, the set identifier 51c that is the same as the set identifier received from the client 61, the item category identifier 51d that is the same as the item category identifier received from the client 61, and the user ID 51e of the user who has logged in, with relevance to each other.

In the client system 6, the client 61 may add the counted value (input value from the input 64) to the value of the total-number-of-transfers information 34 (see FIG. 50) related to the item category identifier 36 that is the same as item category identifier related to the item category name in the item category name display area 92a and overwrite the total-number-of-transfers information 34 after the addition in the storage 62. Furthermore, the client 61 may add the counted value (input value from the input 64) to the value of the total-number-of-transfers information 43 (see FIG. 50) related to the item category identifier 46 and the set identifier 45 that are the same as the set identifier and the item category identifier identical to the ones in the display areas 91 and 92a and overwrite the total-number-of-transfers information 43 in the storage 62. Furthermore, the client 61 may store, in the storage 62, the number-of-transfers information 51a representing the counted value (input value from the input 64), the count-time-instant information 51b representing the time instant at which the counted value is entered, the set identifier 51c that is the same as the set identifier identical to the one in the set name display area 91, the item category identifier 51d that is the same as the item category identifier identical to the one in the item category name display area 92a, and the user ID 51e of the user who has logged in, with relevance to each other.

After medical instruments are delivered to an operating place, physicians and other members perform medical procedures (medical procedures such as surgery, medication, treatment, examination) using these medical instruments. A method of using the management system 1 after such medical procedures is described. Besides, referring to FIG. 53, a flow of processing that the program 29 causes the server 21 to execute and a flow of processing that the program 69 causes the client 61 to execute in using the management system 1 are also described.

First, an operator collects medical instruments in an operating place while sorting out used ones from unused ones. In the following description, a medical instrument that was or has been collected is referred to as a collected tool, an item category to which one or more collected tools belong is referred to as a collected item category, and a set to which one or more collected tools belong is referred to as a collected set. In addition, among the collected tools, a medical instrument that was used is referred to as a used tool and a medical instrument that was not used is referred to as an unused tool.

All medical instruments belonging to a certain set may be unused or used ones in some cases and some of medical members belonging to a certain set may be unused ones in other cases. Thus, the operator checks all of the medical instruments belonging to a set to determine whether they are unused or used ones.

Next, the operator operates the input 64 of the client system 6 at the operating place and logs in to the management system 1 using his own user ID and password. Then, the client 61 causes the display 63 to display a set identifier input screen (set selection screen) (step S11).

Thereafter, the operator enters a set identifier for the collected set via the input 64, and selects a set identifier or a name from several options. As a result, the client 61 gets the set identifier according to the input signal from the input 64 (step S12).

Next, the client 61 requests the server 21 to send information of the collected set by sending the set identifier of the collected set to the server 21 (step S13). Then, the server 21 looks up the set information table 28, retrieves the set name information 28b, the item category identifier 28c, the item category name information 28d, and the quantity information 28e which are related to the set identifier 28a that is the same as the set identifier of the collected set, and sends them to the client 61 (step S71). On the other hand, the client 61 receives and acquires the set name information 28b, the item category identifier 28c, the item category name information 28d, and the quantity information 28e (step S14). It should be noted that the client 61 may look up the set information table 68 in the storage 62 without making a request as step S13. That is, the client 61 may retrieve the set name information 28b, the item category identifier 28c, the item category name information 28d, and the quantity information 28e which are related to the set identifier 28a that is the same as the set identifier of the collected set, from the set information table 68 in the storage 62.

Next, the client 61 allows a screen as shown in FIG. 39 to be displayed on the display 63 according to the set name information 28b, the item category identifier 28c, the item category name information 28d, and the quantity information 28e which have been acquired (step S15). In a set name display area 93 on the screen, a set name according to the set name information 28b is displayed. In a list display area 94 on the screen, item category names according to the item category name information 28d and the quantities according to the quantity information 28e are displayed with relevance to each other. Specifically, item category names are displayed in an item category name display area 94a of the list display area 94 and the quantities are displayed in a quantity display area 94b on the right of the item category name display area 94a.

Then, the operator enters unique identifiers of unused medical instruments one by one using the input 64. In response to this, the client 61 acquires unique identifiers according to the input signals from the input 64 and transmits the acquired unique identifiers to the server 21 with relevance to the set identifier of the collected set (step S16).

The server 21 adds 1 to the value of the total-number-of-non-uses information 35 related to one of the item category identifiers 36 (see FIGS. 42 and 46) every time when it receives a unique identifier from the client 61 (step S73). Specifically, every time when a unique identifier is received, the server 21 looks up the classification table 27 to specify the item category identifier 27a related to the unique identifier 27c that is the same as the received unique identifier and adds 1 to the value of the total-number-of-non-uses information 35 related to the item category identifier 36 that is the same as that item category identifier 27a. Such operations are repeated until the last unique identifier is received. After the increment in response to the reception of the last unique identifier, the summed-up total-number-of-non-uses information 35 is overwritten in the storage 22 by the server 21. Accordingly, the total number of unused instruments is counted up for each collected item category, and the value of the updated total-number-of-non-uses information 35 is a sum. It should be noted that the operation at step S73 may be performed by the client 61. That is, until the last unique identifier is acquired (see step S16), every time when a unique identifier is acquired, the client 61 may look up the classification table 67 to add 1 to the value of the total-number-of-non-uses information 35 (see FIG. 50) of the item category identifier 36 that is the same as the item category identifier 27a related to the unique identifier 27c that is the same as the acquired unique identifier.

Furthermore, the server 21 adds 1 to the value of the total-number-of-non-uses information 44 related to one of the set identifiers 45 and the item category identifiers 46 (see FIGS. 42 and 47) every time when it receives a unique identifier from the client 61 (step S74). Specifically, every time when a unique identifier is received, the server 21 looks up the classification table 27 to specify the item category identifier 27a related to the unique identifier 27c that is the same as the received unique identifier and adds 1 to the value of the total-number-of-non-uses information 44 related to the item category identifier 46 that is the same as that item category identifier 27a and the set identifier 45 that is the same as the received set identifier of the given set (step S74). Such operations are repeated until the last unique identifier is received. After the increment in response to the reception of the last unique identifier, the summed-up total-number-of-non-uses information 44 is overwritten in the storage 22 by the server 21. Accordingly, the total number of the collected unused instruments is counted up for each collected item category belonging to a collected set, and the value of the updated total-number-of-non-uses information 44 is a sum. It should be noted that the operation at step S74 may be performed by the client 61. That is, until the last unique identifier is acquired (see step S16), every time when a unique identifier is acquired, the client 61 may look up the classification table 67 in the storage 62 to specify the item category identifier 27a related to the unique identifier 27c that is the same as the acquired unique identifier and add 1 to the value of the total-number-of-non-uses information 44 related to the item category identifier 46 that is the same as that item category identifier 27a and the set identifier 45 that is the same as the set identifier of the given set (see FIG. 50).

The server 21 adds 1 to the value of the non-use-number-of-times information 32 related to the unique identifier 33 that is the same as the unique identifier received from the client 61 (see FIG. 45) and overwrites the non-use-number-of-times information 32 in the storage 22 (step S75). Accordingly, the number of times the collected instruments were not used is counted up and the value of the updated non-use-number-of-times information 32 is a sum. It should be noted that the operation at step S75 may be performed by the client 61. That is, after the acquisition of the unique identifier (step S16), the client 61 may add 1 to the value of the non-use-number-of-times information 32 (see FIG. 50) related to the unique identifier 33 that is the same as the acquired unique identifier and overwrite the non-use-number-of-times information 32 in the storage 62.

As described above, every time when a unique identifier is received, the item category identifier 27a related to the unique identifier 27c that is the same as the received unique identifier is specified by looking up the classification table 27. The server 21 calculates the number of times it specifies the item category identifiers 27a from the reception of the first unique identifier to the reception of the last unique identifier, for each item category identifier 27a. Such a calculated value for each item category identifier 27a is equal to the number of the unused medical instruments for each item category. The server 21 stores, in the storage 22, the number-of-non-uses information 52a representing that calculated value, the count-time-instant information 52b representing the time instant at which the last unique identifier was received, the set identifier 52c that is the same as the set identifier received from the client 61, the item category identifier 51d that is the same as the specified item category identifier 27a, and the user ID 51e of the user who has logged in, with relevance to each other as shown in FIG. 49 (step S76). Here, the unused information 52a etc. stored at step S76 is a log for a single processing shown in FIG. 53. The number-of-non-uses information 52a etc. are accumulated by repeating the processing shown in FIG. 53, which are the number-of-non-uses log data 52. It should be noted that the operation at step S76 may be performed by the client 61. That is, the client 61 may calculate the number of times the item category identifiers 27a are specified from the acquisition of the first unique identifier to the acquisition of the last unique identifier for each item category identifier 27a. In such a case, the client 61 stores, in the storage 62, the number-of-non-uses information 52a, the count-time-instant information 52b, the set identifier 52c, the item category identifier 52d, and the user ID 52e, with relevance to each other as the number-of-non-uses log data 52.

Next, when all medical instruments belonging to a set are unused, the operator enters the set identifier of the collected set using the input 64 or selects the set identifier or the name from some choices. As a result, the client acquires the set identifier according to the input signal from the input 64 and sends it to the server 21 (step S17). Then, the server 21 adds 1 to the value of the non-use-number-of-times information 42 (see FIGS. 42 and 47) related to the set identifier 45 that is the same as the set identifier (set identifier received from the client 61) of the collected set and overwrites the non-use-number-of-times information 42 in the storage 22, with relevance to the set identifier 45 (step S77). It should be noted that, in the client system 6, the client 61 may add 1 to the value of the non-use-number-of-times information 42 (see FIG. 50) related to the set identifier of the collected set and overwrites that non-use-number-of-times information 42 in the storage 62.

In the aforementioned preferred embodiments, the values of the total-number-of-non-uses information 35 and 44 for each given item category are incremented in the server 21 or the client 61 (see steps S73 and step S74) in response to the operator's successive entries of the unique identifiers of the given medical instruments using the input 64 (see step S16), and the number-of-non-uses information 52a for each given item category is stored (see step S76).

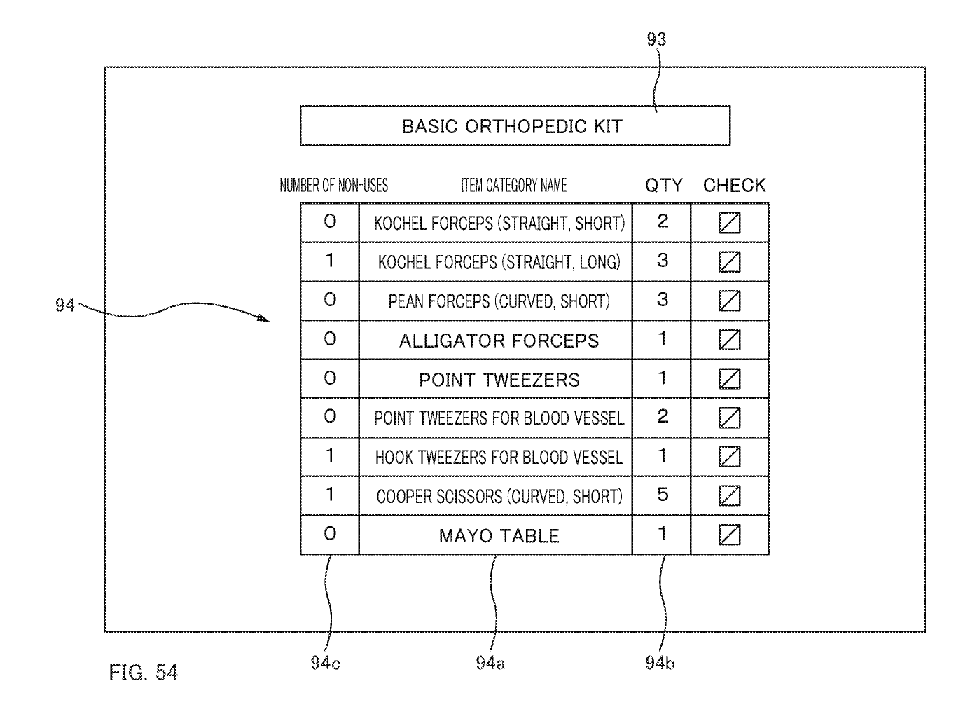

On the other hand, the operator may count the number of given medical instruments for each given item category belonging to a given set and enter a counted value for each given item category using the input 64. For example, when the operator selects (e.g., clicks or taps) the item category name display area 94a depending on the number of times equal to the counted value or selects the item category name display area 94a once and then enters a numerical value of the counted value by operating the input 64 in a screen shown in FIG. 54, the entered counted value is displayed in a NUMBER-OF-NON-USES display area 94c on the left of the item category name display area 94a. As a result, the client 61 acquires a counted value for each given item category according to the input signals from the input 64. The client 61 then sends, the acquired counted value to the server 21 with relevance to the item category identifier (which is the same as the item category identifier 28c acquired at step S14) and the set identifier (which is the same as the set identifier acquired at step S12). Thereafter, the server 21 adds the counted value to the value of the total-number-of-non-uses information 35 (see FIGS. 42 and 46) related to the item category identifier 36 (see FIG. 46) that is the same as the item category identifier received from the client 61 and overwrites the total-number-of-non-uses information 35 after the addition in the storage 22. Furthermore, the server 21 adds the counted value to the value of the total-number-of-non-uses information 44 see FIGS. 42 and 47) related to the item category identifier 46 and the set identifier 45 (see FIG. 47) that are identical to the item category identifier and the set identifier received from the client 61 and overwrites the total-number-of-non-uses information 44 after the addition in the storage 22.

Furthermore, the server 21 stores, in the storage 22, the number-of-non-uses information 52a representing the counted value received from the client 61, the count-time-instant information 52b representing the time instant at which the counted value is received from the client 61, the set identifier 52c that is the same as the set identifier received from the client 61, the item category identifier 52d that is the same as the item category identifier received from the client 61, and the user ID 52e of the user who has logged in, with relevance to each other.

In the client system 6, the client 61 may add the counted value (input value from the input 64) to the value of the total-number-of-non-uses information 35 (see FIG. 35) related to the item category identifier 36 that is the same as item category identifier related to the item category name in the item category name display area 94a and overwrite the total-number-of-non-uses information 35 after the addition in the storage 62. Furthermore, the client 61 may add the counted value (input value from the input 64) to the value of the total-number-of-non-uses information 44 (see FIG. 50) related to the item category identifier 46 and the set identifier 45 that are the same as the set identifier and the item category identifier identical to the ones in the display areas 93 and 94a and overwrite the total-number-of-non-uses information 44 in the storage 62. Furthermore, the client 61 may store, in the storage 62, the number-of-non-uses information 52a representing the counted value (input value from the input 64), the count-time-instant information 52b representing the time instant at which the counted value is entered, the set identifier 52c that is the same as the set identifier identical to the one in the set name display area 93, the item category identifier 52d that is the same as the item category identifier identical to the one in the item category name display area 94a, and the user ID 52e of the user who has logged in, with relevance to each other.

With the functions of the management system 1 as described above, the number of times each medical instrument has been transferred and the number of times each medical instrument was not used are tallied up for each medical instrument (see FIG. 45), the total number of transfers and the total number of non-uses are tallied up for each item category (see FIG. 46), the total number of transfers and the total number of non-uses are tallied up for each set and item category (see FIG. 47), and logs of the number of transferred ones and the number of unused ones are tallied up (FIGS. 48 and 49). This management system 1 has a function of calculating a percentage of non-usage using tallied information 31, 32, 34, 35, 41, 42, 43, and 44 as well as data 51 and 52.

The server 21 calculates a percentage of non-usage by dividing the value of the non-use-number-of-times information 32 by the value of the transfer-number-of-times information 31 for each unique identifier 33. The calculated percentage of non-usage is a percentage of non-usage for each medical instrument. Thereafter, the server 21 allows the calculation result for each unique identifier 33 to be displayed on the display 23 with relevance to the unique identifier 33.

The server 21 also calculates a percentage of non-usage by dividing the value of the total-number-of-non-uses information 35 by the value of the total-number-of-transfers information 34 for each item category identifier 36. The calculated percentage of non-usage is a percentage of non-usage for each item category. Thereafter, the server 21 allows the calculation result for each item category identifier 36 to be displayed on the display 23 with relevance to the item category identifier 36 or an item category name (the item category name is obtained as the item category name information 27b by looking up the classification table 27).

The server 21 also calculates a percentage of non-usage by dividing the value of the non-use-number-of-times information 42 by the value of the transfer-number-of-times information 41 for each set identifier 45. The calculated percentage of non-usage is a percentage of non-usage for each set. Thereafter, the server 21 allows the calculation result for each set identifier 45 to be displayed on the display 23 with relevance to the set identifier 45 or a set name (the set name is obtained as the set name information 28b by looking up the set information table 28).

Furthermore, the server 21 calculates a percentage of non-usage by dividing the value of the total-number-of-non-uses information 44 by the value of the total-number-of-transfers information 43 for each set identifier 45 and item category identifier 46. The calculated percentage of non-usage is a percentage of non-usage of the item category for each set and each item category. Thereafter, the server 21 allows the calculation result for each set identifier 45 and item category identifier 46 to be displayed on the display 23 with relevance to the item category identifier 46 or an item category name (the item category name is obtained as the item category name information 28d by looking up the set information table 28) and the set identifier 45 or a set name (the set name is obtained as the set name information 28b by looking up the set information table 28).

Furthermore, the server 21 reads the number-of-transfers log data 51 and sums up the values of the number-of-transfers information 51a related to the same item category identifier 51d. The server 21 also reads the number-of-non-uses log data 52 and sums of the values of the number-of-non-uses information 52a related to the same item category identifier 52d. The server 21 then calculates a percentage of non-usage by dividing the sum of the number-of-non-uses information 52a by the sum of the number-of-transfers information 51a for the same item category identifiers 51d and 52d. The calculated percentage of non-usage is a percentage of non-usage for each item category. Thereafter, the server 21 allows the calculation result for the same item category identifiers 51d and 52d to be displayed on the display 23 with relevance to the item category identifiers 51d and 52d or an item category name (the item category name is obtained as the item category name information 28d by looking up the set information table 28).

The server 21 reads the number-of-transfers log data 51 and sums up the values of the number-of-transfers information 51a related to the same set identifier 51c and the same item category identifier 51d. The server 21 also reads the number-of-non-uses log data 52 and sums up the values of the number-of-non-uses information 52a related to the same set identifier 52c and the same item category identifier 52d. Then, the server 21 calculates a percentage of non-usage for the same set identifiers 51c and 52d as well as the same item category identifiers 51d and 52d by dividing the sum of the number-of-non-uses information 52a by the sum of the number-of-transfers information 51a. The calculated percentage of non-usage is a percentage of non-usage of the item category for each set and each item category. Thereafter, the server 21 allows the calculation result for the same item category identifiers 51d and 52d to be displayed on the display 23 with relevance to the item category identifiers 51d and 52d or an item category name (the item category name is obtained as the item category name information 28d by looking up the set information table 28) and the set identifiers 51c and 52d or a set name (the set name is obtained as the set name information 28b by looking up the set information table 28).

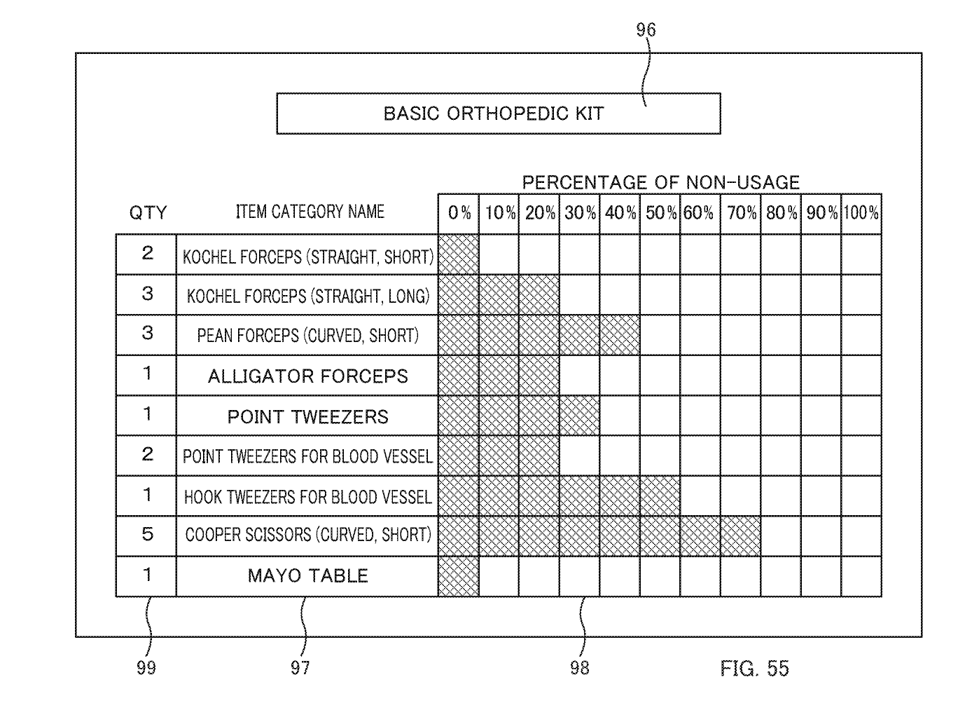

Each calculation result is displayed by, for example, a numerical value (text), symbol, mark, pattern, or indicator. FIG. 55 is an example of a display screen for the calculation result. As shown in FIG. 55, the set name (the set name is obtained as the set name information 28b by looking up the set information table 28) corresponding to the set identifier 45 is displayed in a set name display area 96 on the screen. In addition, item category names (each item category name is obtained as the item category name information 28d by looking up the set information table 28) corresponding to the item category identifier 46 is displayed in an item category name display area 97. A percentage of non-usage (a ratio expressed as a fraction of 100) obtained by dividing the value of the total-number-of-non-uses information 44 by the value of the total-number-of-transfers information 43 is displayed as an indicator in a percentage-of-non-usage display 98 on the right of the item category name display area 97. The number of medical instruments belonging to a set and an item category (the number of them is obtained as the quantity information 28e by looking up the set information table 28) is displayed in a quantity display area 99 on the left of the item category name display area 97.

It should be noted that, with the percentage of non-usage calculated by the server 21, the calculation result may be transmitted to the client 61 and the client 61 may allow the calculation result to be displayed on the display 63. Alternatively, in a manner similar to the one how the server 21 calculates the percentages of non-usage, the client 61 may read the pieces of information 31, 32, 34, 35, 41 to 44 and the data 51 and 52 from the storage 62 and calculate each percentage of non-usage, and the client 61 may allow the calculation result to be displayed on the display 63.

According to the preferred embodiments described above, the following advantageous effects are achieved.

Since the classification table 27 is stored in the storage 22, it becomes possible to accurately manage classifications of medical instruments for each item category using the server 21.

Furthermore, since the set information table 28 is stored in the storage 22, it becomes possible to accurately manage grouping of item categories for each set using the server 21. Even when medical instruments belonging to the same item category are interchanged between different sets, the management by the server 21 is not affected.

Accordingly, it is possible to standardize classifications for medical instruments for each item category in a hospital and avoid excessive stock of medical instruments in the entire hospital.

As shown in FIG. 45, since the number of times (sum) each medical instrument was not used is stored in the storage 22 or the storage 62 (see step S75) for each medical instrument, it is possible to easily and accurately manage the number of times each medical instrument was not used.

As shown in FIG. 46, since the total number of unused medical instruments (sum) is stored in the storage 22 or the storage 62 (step S75) for each item category, such total number can be managed easily and accurately.

As shown in FIG. 47, since the number of times (sum) each set was not used is stored in the storage 22 or the storage 62 (step S77) for each set, it is possible to easily and accurately manage the number of times each set was not used.

As shown in FIG. 47, since the total number of unused medical instruments for each set and item category (sum) is stored in the storage 22 and the storage 62 (step S74), it is possible to easily and accurately manage such total number for each set and item category.

Since the number of unused medical instruments for each transferred item category is stored as a log of the number-of-non-uses log data 52 in the storage 22 or the storage 62 with relevance to a time instant and a user ID, it is possible to find when and who counts the number of unused medical instruments.

For example, although the non-use number of times, the number of unused ones, and the total number of non-uses are counted in the aforementioned preferred embodiments, the number of used times, the number of used ones, and the total number of used ones may be counted. In such cases, the term “unused” should read “used” and the term “used” should read “unused” in the above description of the preferred embodiments except for the definition of the used and unused tools (among the collected tools, a used medical instrument is referred to as a used tool and an unused medical instrument is referred to as an unused tool).

### Sixth Preferred Embodiment

A sixth preferred embodiment of the present invention relates to implant management systems and non-transitory computer readable media including implant management programs.

When a medical procedure such as a surgery or therapy is performed on a patient, it is common to use two or more medical instruments.

Of these, medical instruments owned by a medical facility can be managed electronically based on identifiers (two-dimensional symbols) attached thereto which are unique to each medical instrument (individual instruments included in the medical instrument). Alternatively, even medical instruments to which no identifier can be attached can be managed using, for example, an indication such as a tape.

As medical instruments used for medical procedures, some are on loan from a manufacturer (medical instruments borrowed from an outside, hereinafter also referred to as a “borrowed instrument”). Instruments on loan are, for example, special medical instruments dedicated to a specific surgery. Such special medical instruments are not frequently used and thus medical facilities usually do not have their own due to lack of a storage space and management cost. They usually borrow such instruments from a manufacturer as necessary, and return them after use.

Medical facilities are not permitted to attach unique identifiers or marks to such borrowed instruments. Accordingly, they are managed using paper-based borrowing slips and shipping slips created by the manufacturer.

However, such slips contain only names of borrowed instruments, it is difficult to determine whether the borrowed instruments that a medical facility keeps are the exact ones indicated in the slips or whether no instrument included in the borrowed instruments has been missing. Therefore, management of borrowed instruments by medical facilities often tend to be insufficient.

JP-A-2002-132927 discloses a technique of managing medical articles provided by a supplier of medical articles to a medical facility using a communication network between the medical facility and the supplier of medical articles used in the medical facility.

Now, some borrowed instruments include one or more implants. Implants are instruments that are placed in a patient's body during a medical procedure, such as artificial joints, mechanical valves for the heart and stents that is put in a bile duct to keep it wider. Different kinds of implants may be included in a single borrowed instrument. Alternatively, a single implant may include a plurality of similar items (e.g., two or more artificial joints having different sizes).

Physicians who perform surgery selects an implant necessary for a patient from a plurality of implants (items included in the implant) and places it in the patient's body. Since such implants (especially the items included in the implants) are similar in shape and name, it is difficult to manage them with the paper-based slip alone.

Furthermore, one or more of the implants (items included in the implants) are placed in the body, the number of the inventory differs before and after a medical procedure. In managing implants, it is necessary to know such a difference in number. This makes managements harder compared to those for ordinary medical instruments.

A preferred embodiment of the present invention provide systems for managing implants included in a borrowed instrument.

An implant management system according to a preferred embodiment of the present invention includes an identifier that identifies, based on an input of information regarding a borrowed instrument, the borrowed instrument being a medical instrument borrowed from an outside, whether the borrowed instrument includes an implant; a display controller that allows, in the case that the borrowed instrument includes an implant, a registration screen to be displayed, the registration screen being used to register the implant; and an instrument register that registers an image of the implant selected on the registration screen with association to the borrowed instrument that includes the implant.

With this preferred embodiment, it is possible to manage implants included in borrowed instruments.

In the descriptions of the specification and the drawings that follow, at least the following features are disclosed in addition to the aforementioned preferred embodiments of the present invention.

That is, the implant management system in which the implant contains two or more items, and the instrument register registers an image of the items and a number of the items with association to the borrowed instrument that includes the implant is disclosed. Such a system makes it possible to manage items included in implants.

Furthermore, the implant management system further including a counter that performs counting of the borrowed instrument according to a command input, in which the display controller allows the image of the items related to the borrowed instrument to be displayed after the use of the borrowed instrument, and the counter is incremented for the items based on a choice of the image of the items is disclosed. With such a system, it is possible to know the state of usage of the items included in implants.

Furthermore, the implant management system in which the instrument register registers an identification information to identify the items with association to the image of the items, and the display controller allows the identification information to be displayed along with the image of the items is disclosed. Such a system makes it possible to easily distinguish items even when one implant includes a plurality of similar items.

Furthermore, the implant management system further including a specifier that specifies a used item based on a photographed image obtained by photographing the item that has not been used and the image of the items, in which the display controller allows the image of the specified item and an image of other items to be displayed in a different mode is disclosed. With such a system, it is possible to easily know items that has actually been used among a plurality of items.

Furthermore, the implant management system in which the instrument register registers a user information of a user who has performed the registration of the implant or the items with association to the image of the implant is disclosed. With such a system, it is possible to manage operators who has performed registration of implants or items as well.

Furthermore, an implant management system includes a display controller that allows a first registration screen to register a borrowed instrument to be displayed, the borrowed instrument being a medical instrument borrowed from an outside; an instrument register that registers a borrowed instrument selected the first registration screen; and an identifier that identifies whether the borrowed instrument includes an implants based on an input of information regarding the borrowed instrument, in which the display controller allows, in the case that the borrowed instrument includes an implant, a second registration screen to be displayed, the second registration screen being used to register the implant, the instrument register registers an image of the implant selected on the second registration screen with association to the borrowed instrument that includes the implant is disclosed. With such a system, it is possible to manage borrowed instruments and implants included in the borrowed instruments.

Furthermore, a non-transitory computer readable medium including a program that causes a computer to identify, based on an input of information regarding a borrowed instrument, the borrowed instrument being a medical instrument borrowed from an outside, whether the borrowed instrument includes an implants; allow, in the case that the borrowed instrument includes an implant, a registration screen to be displayed, the registration screen being used to register the implant; and register an image of the implant selected on the registration screen with association to the borrowed instrument that includes the implant is disclosed. With such a program, it is possible to manage implants included in borrowed instruments.

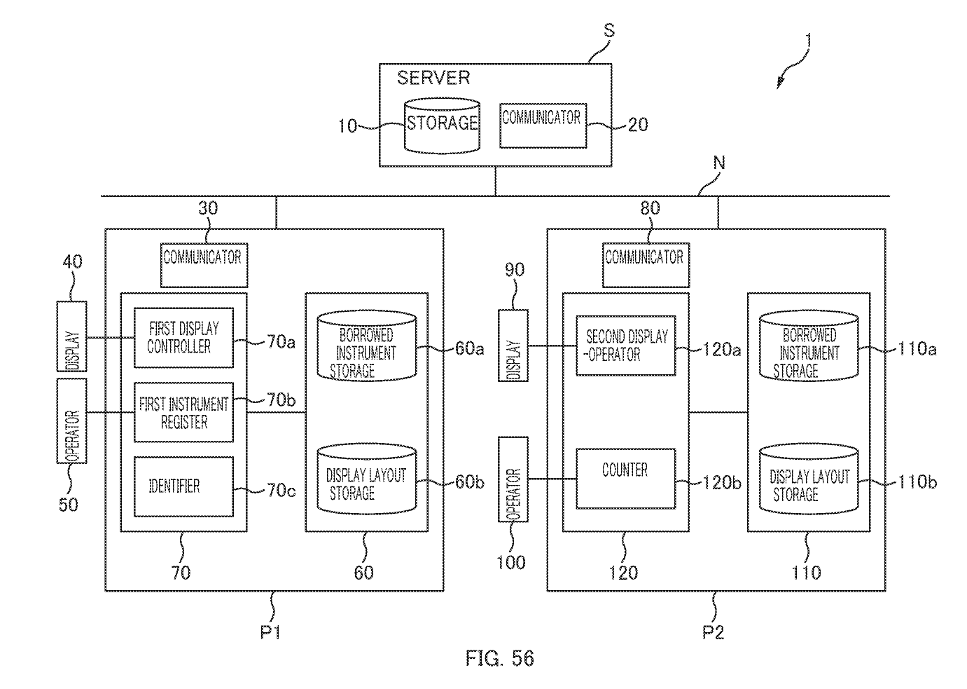

Referring to FIGS. 56 to 66B, an implant management system according to a sixth preferred embodiment (hereinafter, also referred to as a “system 1”) is described.

As shown in FIG. 56, the system 1 of this preferred embodiment includes terminals (in this example, two terminals: a “terminal P1” and a “terminal P2”), and a server S. The terminals and the server S are connected via a network N such as an intranet, a local area network (LAN), or the Internet so that they can communicate with each other via cables or wirelessly. Each terminal is a desktop personal computer or a mobile terminal (such as a laptop PC and a tablet computer).

The server S is a computer that accumulates and manages various kinds of information as well as exchanges information with the terminals. The server S of the present preferred embodiment has a function of interfacing the exchange of information between or among the terminals. The server S can be installed in a hospital facility where each terminal is located or installed in a remote location such as a server provider and the like.

The server S preferably includes a storage 10 and a communicator 20. The storage 10 (server-side storage) is a large-capacity storage that stores, for example, information regarding medical instruments. The storage 10 can store, for example, data related to borrowed instruments (described later) transmitted from the terminals. The communicator 20 exchange data with the terminals via the network N.

The terminals P1 and P2 are devices that display borrowed instruments used for medical procedures and sending and receiving information to and from the server S. The terminals can be located at different areas in a hospital facility. For example, the terminal P1 is located in an area where borrowed instruments before use are stored and the terminal P2 is located in an area where used borrowed instruments are collected. In this preferred embodiment, the term “before the use (after the use) of a borrowed instrument” is equivalent to “before a medical procedure (after a medical procedure) (for which the medical instrument is used).”

The terminal P1 of the present preferred embodiment functions as a device to determine whether one or more implants are included in a borrowed instrument and, if any, register an image of the implant with relevance to the borrowed instrument. The terminal P1 preferably includes a communicator 30, a storage 60, and a controller 70, and is connected to a display 40 and an operator 50.

The communicator 30 exchange data with the server S and the terminal P2 via the network N. The display 40 displays, for example, images of borrowed instruments and implants. The display 40 and the terminal P1 may be one unit. The operator 50 is an input interface such as a mouse. Alternatively, the display 40 that is a touch-panel screen may double as the operator 50. The operator 50 and the terminal P1 may be one unit. An operator enters commands to the terminal P1 via the operator 50.

The storage 60 is a large-capacity storage that stores various kinds of data. As shown in FIG. 56, in this preferred embodiment, a borrowed instrument storage 60a and a display layout storage 60b are portions of a storage region of the storage 60.

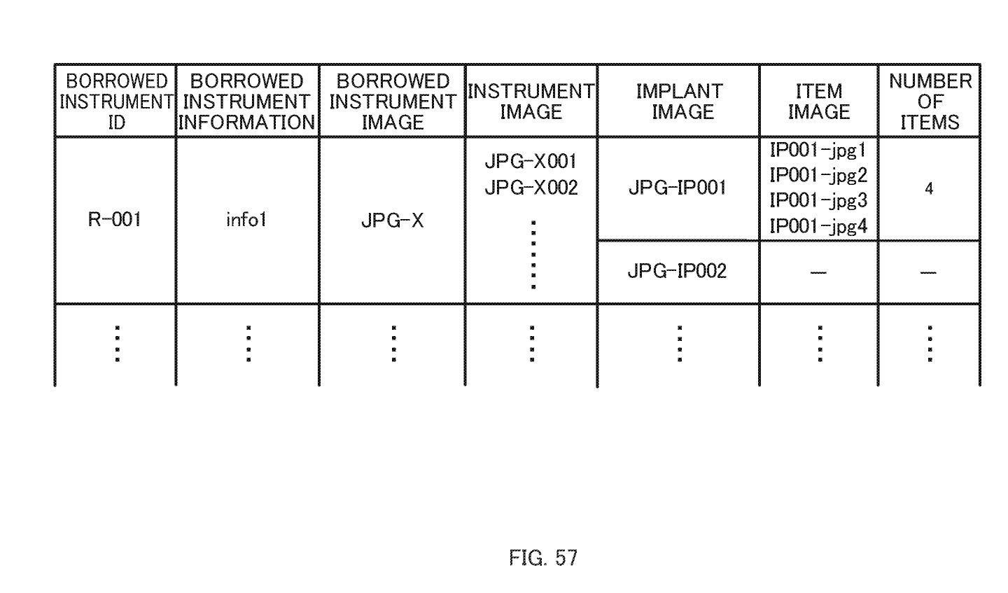

The borrowed instrument storage 60a stores data about borrowed instruments. FIG. 57 is an example of data stored in the borrowed instrument storage 60a. As shown in FIG. 57, for each borrowed instrument, identification information (borrowed instrument ID), borrowed instrument information (described later), image data for borrowed instruments (borrowed instrument images), image data of individual instruments included in borrowed instruments (instrument images), image data of implants included in borrowed instruments (implant images), image data of items included in implants (item images), and the number of items are stored with relevance to each other. Various image data are data for displaying various instruments on the display 40.

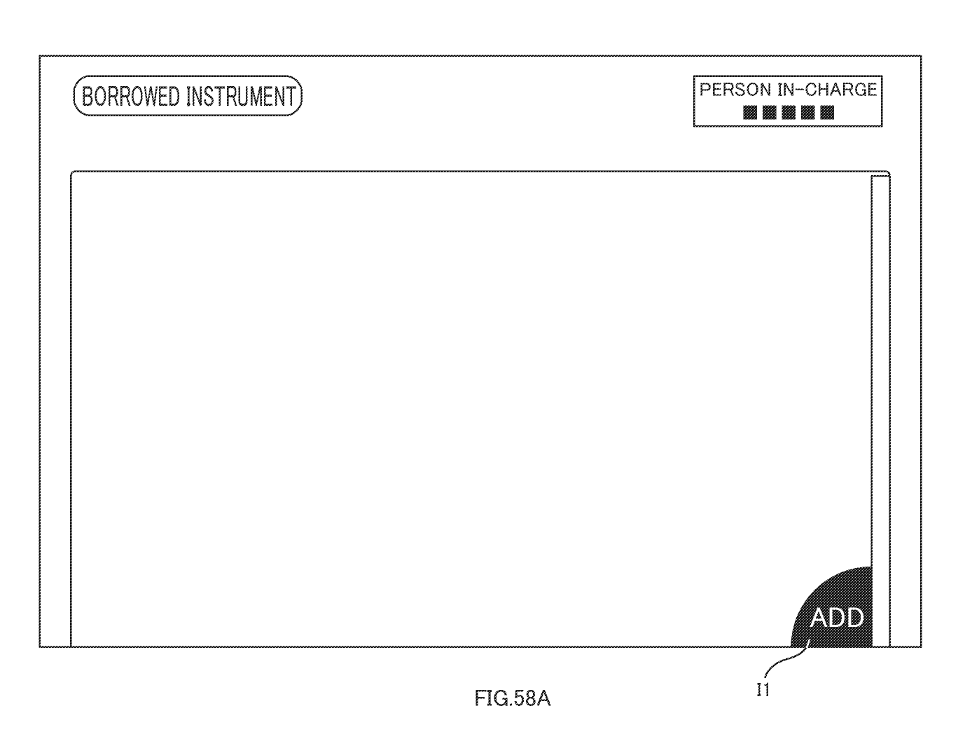

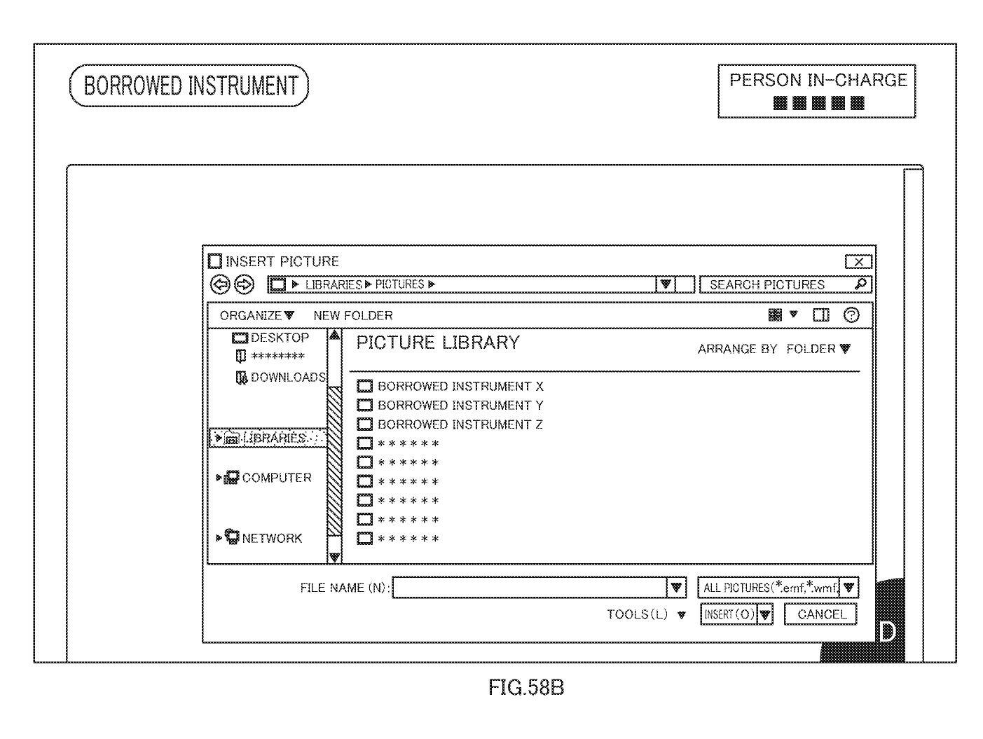

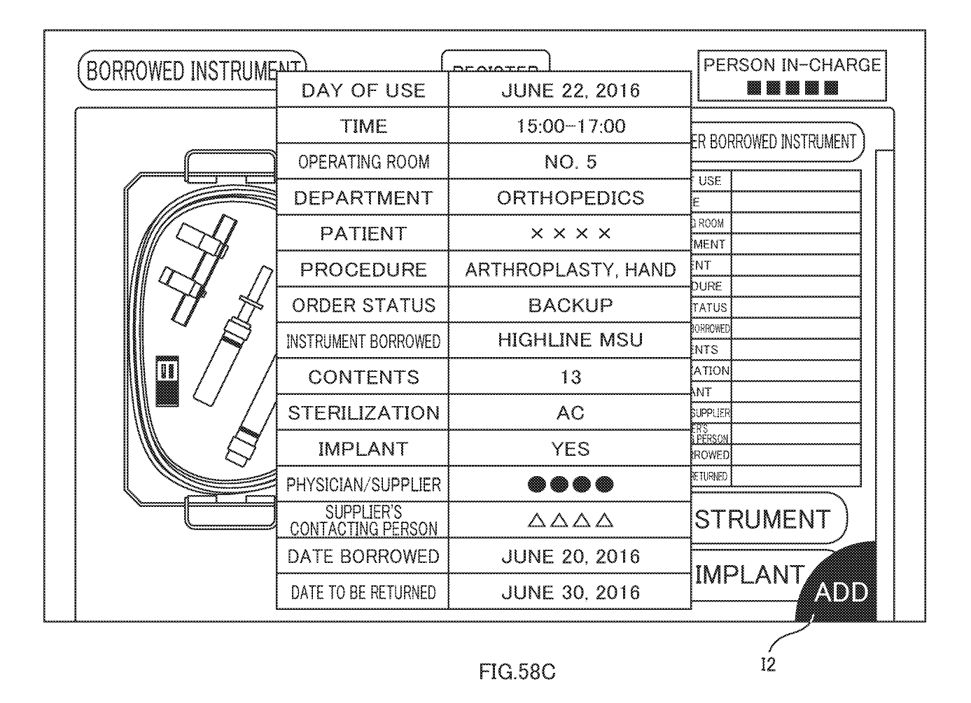

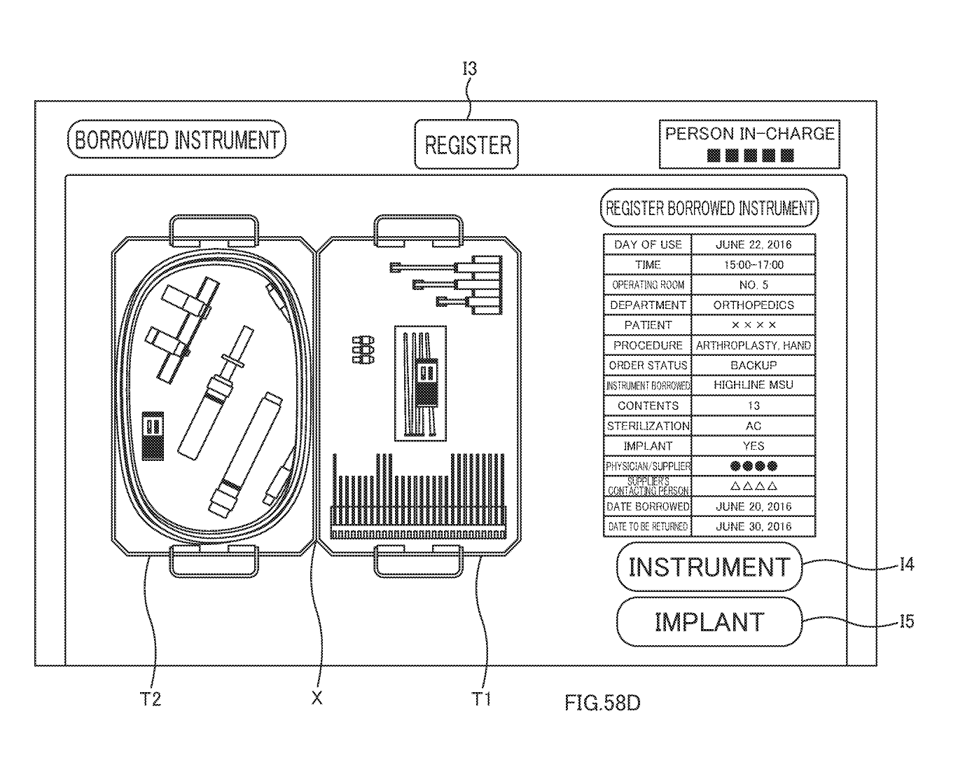

The display layout storage 60b has layout data for various display screens (see, for example, FIG. 58A). A first display controller 70a (described later) provides display screens on the display 40 based on the layout data. It should be noted that the layout data may be stored in, for example, the storage 10 of the server S.

The controller 70 preferably includes a CPU and a memory (which are not shown). The CPU achieves different kinds of control functions by executing an operating program stored in the memory. The memory is a storage that stores a program or programs executed by the CPU or temporarily stores various pieces of information upon execution of the program(s). The controller 70 executes, based on signals (command inputs) from the operator 50, corresponding processing operations.

The controller 70 of the present preferred embodiment defines and functions as the first display controller 70a, an instrument register 70b, and an identifier 70c.

The first display controller 70a performs various display controls in the terminal P1.

The first display controller 70a of the present preferred embodiment allows a registration screen (first registration screen) to register borrowed instruments to be displayed on the display 40. The first display controller 70a also allows a registration screen (second registration screen) to register, in the case that a borrowed instrument includes an implant, the implant to be displayed. Details of these registration screens are described later.

Each registration screen is displayed based on layout data stored in the display layout storage 60b. The first display controller 70a is an example of the “display controller.”

The instrument register 70b performs processing of registering a borrowed instrument and an implant included in the borrowed instrument to the system 1.

The instrument register 70b also registers the borrowed instrument selected in the first registration screen. Furthermore, the instrument register 70b registers an image of the implant selected in the second registration screen with association to the borrowed instrument in which the implant is included.

If an implant includes a plurality of items, the instrument register 70b may register images of the items and the number of the items with association to the borrowed instrument in which the implant is included.

In this case, the instrument register 70b may register identification information to identifying items with association to the images of the items. Items included in one implant are only slightly different in size or shape from each other and are often hard to distinguish at a glance. Thus, the instrument register 70b registers, with association to the images of the items, information (identification information) that makes easy to distinguish these items. The identification information is information to distinguishing items from each other such as the length, diameter, and weight of each item. The identification information is registered by an operator entering it in the second registration screen using the operator 50.

Furthermore, the instrument register 70b can register user information of a user (e.g., the name or ID of an operator) who has performed the registration of the implant or the item with association to the image of the implant. The operator who has performed counting can be identified using, for example, a log-in status to the terminal.

It should be noted that, in this preferred embodiment, the registration of the borrowed instruments etc. is equivalent in meaning to store images etc. of the borrowed instruments in the borrowed instrument storage 60a. The instrument register 70b may send data related to the borrowed instruments to the server S via the communicator 30 to allow it to be stored in the storage 10.

The identifier 70c determines, based on an input of information regarding a borrowed instrument, whether the borrowed instrument includes an implant.

In registering a borrowed instrument, an operator enters information regarding the borrowed instrument in the registration screen. The identifier 70c refers the information thus entered and determines whether an implant is present. If any, the identifier 70c sends a signal indicative of this to the first display controller 70a. If this signal is received, the first display controller 70a allows the registration screen (second registration screen) to register implants to be displayed according to a command input from the operator.

Here, referring to FIGS. 58A to 62B, a specific example of processing performed in the terminal P1 from the registration of a new borrowed instrument X to the display after the registration is described. FIGS. 58A to 62B show screens displayed on the display 40. It is assumed that the operator enters various inputs (selections) via the operator 50.

The new borrowed instrument X includes a plurality of instruments and implants (an implant p1, an implant p2). The borrowed instrument X has two trays (trays T1 and T2) and instruments and implants are placed in each tray. The implant p1 includes a plurality of items (items e1 to e4). Furthermore, it is assumed that images of the borrowed instrument X etc. are photographed beforehand using an image-pick up device and stored in the storage 60. It is assumed that the image of the borrowed instrument X, images of the instruments included in the borrowed instrument X, images of the trays in which the instruments are placed, images of the implants, images of the items are stored in different folders in the storage 60.

When a registering operator logs in to the system 1 via the terminal P1, the first display controller 70a allows a registration screen for a borrowed instrument to be displayed (see FIG. 58A). When the operator selects an “add” icon I1, the first display controller 70a allows a folder in which an image related to the borrowed instrument is included to be displayed (see FIG. 58B). The operator selects the image of the borrowed instrument X stored in the folder.

The first display controller 70a allows the image of the selected borrowed instrument X to be displayed on the display 40 (see FIGS. 58C and 58D). Furthermore, the first display controller 70a allows an entry field to enter information about with the borrowed instrument X to be displayed on an image (see FIG. 58C). The operator enters necessary information into the entry field. In this example, the expected date when the borrowed instrument X is used (expected date of use), the time of use, the room where it is used (operating room), the department, the patient name, the procedure, the arrangement status, the name of the borrowed instrument, and the number of instruments included in the borrowed instrument (the number of instruments), the sterilization method, the presence or absence of an implant, the name of a physician/supplier, the supplier's contacting person, the date borrowed, the expected return date are written.

When the entry of the information about the borrowed instrument X is completed, the operator selects an icon I2. The first display controller 70 allows an image of the borrowed instrument X and the entered information about the borrowed instrument X (see FIG. 58 D) to be displayed. When the operator selects a “register” icon I3, the instrument register 70b registers the information regarding the borrowed instrument X and the image of the borrowed instrument X in a medical instrument storage 60a. FIGS. 58A to 58D are examples of the “first registration screen”.

The borrowed instrument X includes a plurality of instruments. In this case, these instruments can also be registered separately. Here, an example of registration of the tray T1 in which a plurality of instruments including an implant are arranged is described.

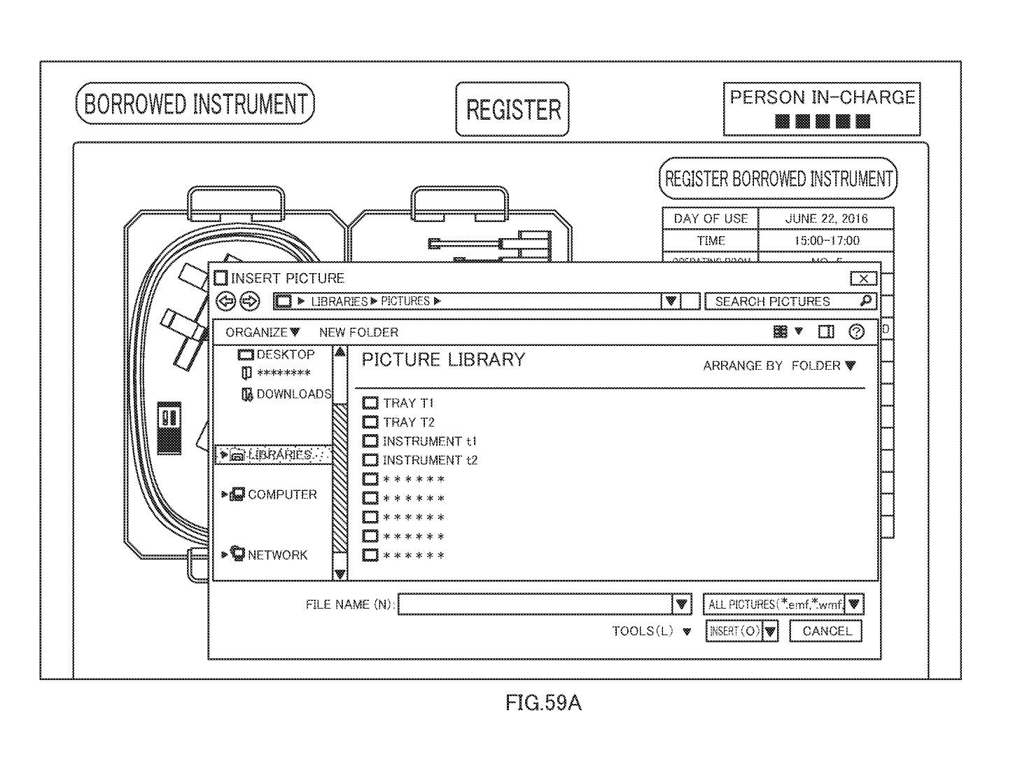

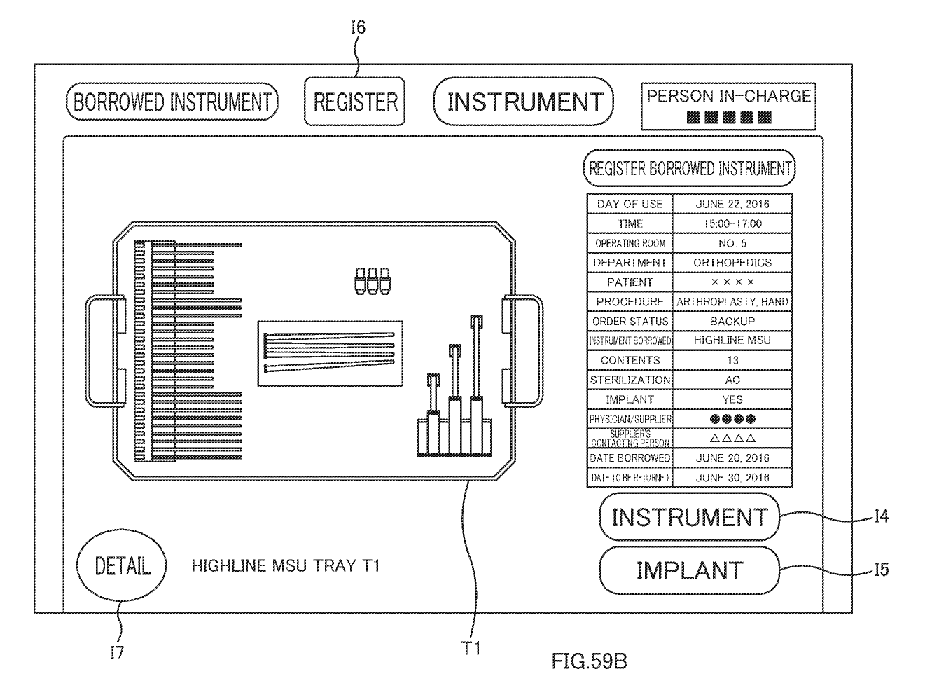

When registering instruments, the operator selects an “instrument” icon I4 in the registration screen shown in FIG. 58D. The first display controller 70a allows a folder including images related to the instrument to be displayed (see FIG. 59A). The operator selects the image of the tray T1 included in the borrowed instrument X from the folder. The first display controller 70 a allows the image of the selected tray T1 to be displayed on the display 40 (see FIG. 59B). When the operator selects a “register” icon I6, the instrument register 70b registers the tray T1 in the medical instrument storage 60a in association with relevance to the instrument X.

It should be noted that it is also possible to register images of individual instruments included in the tray T1 with association to the borrowed instrument X. In this case, the operator selects a “detail” icon I7. As in FIG. 59A, the first display controller 70a allows folders of images regarding the instrument to be displayed. Then, the operator selects an image of the individual instruments (for example, the instrument t1 and the instrument t2 shown in FIG. 59A) included in the tray T1 from the folder and selects the “register” icon I6, thereby registering the image of the instrument.

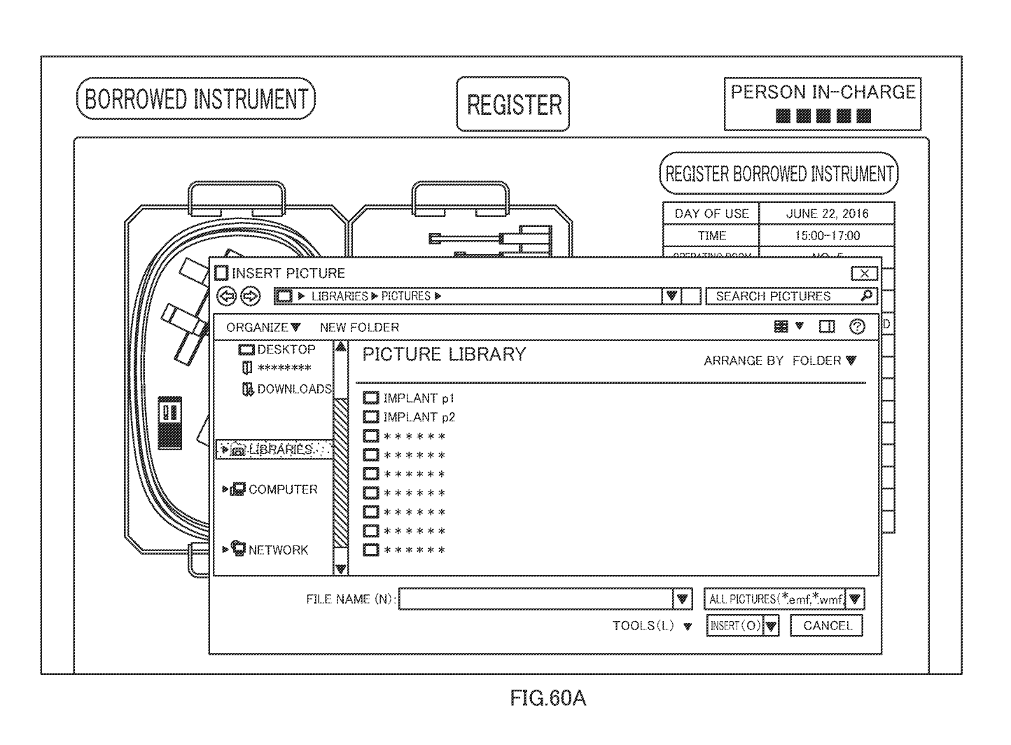

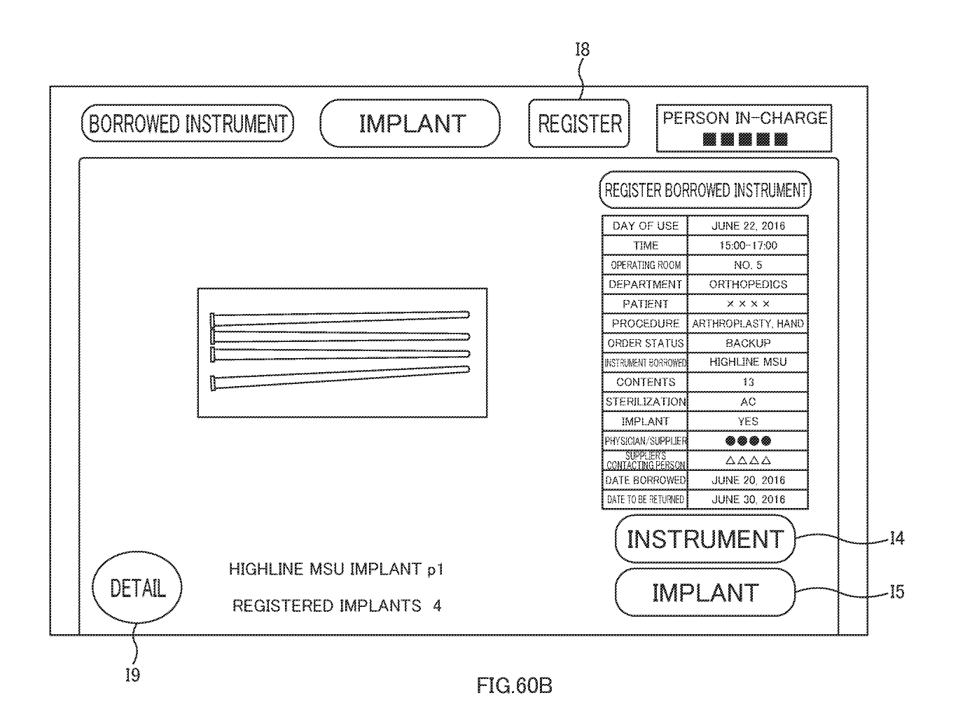

When registering an implant, the operator selects an “implant” icon I5 in the registration screen shown in FIG. 58D. The first display controller 70a allows a folder of the images related to the implant to be displayed (see FIG. 60A). The operator selects the image of the implant p1 included in the borrowed instrument X from the folder. The first display controller 70a allows the image of the selected implant p1 to be displayed in the display 40 (see FIG. 60B). When the operator selects a “register” icon I8, the instrument register 70b registers the image of the implant p1 with association to the borrowed instrument X in the medical instrument storage 60a. When the implant p1 includes a plurality of items, the operator can enter the number of items in the registration screen. FIG. 60B shows a state in which the number (4) of the items e1 to e4 included in the implant p1 is filled in. FIGS. 60A and 60B are examples of the “second registration screen 110.”

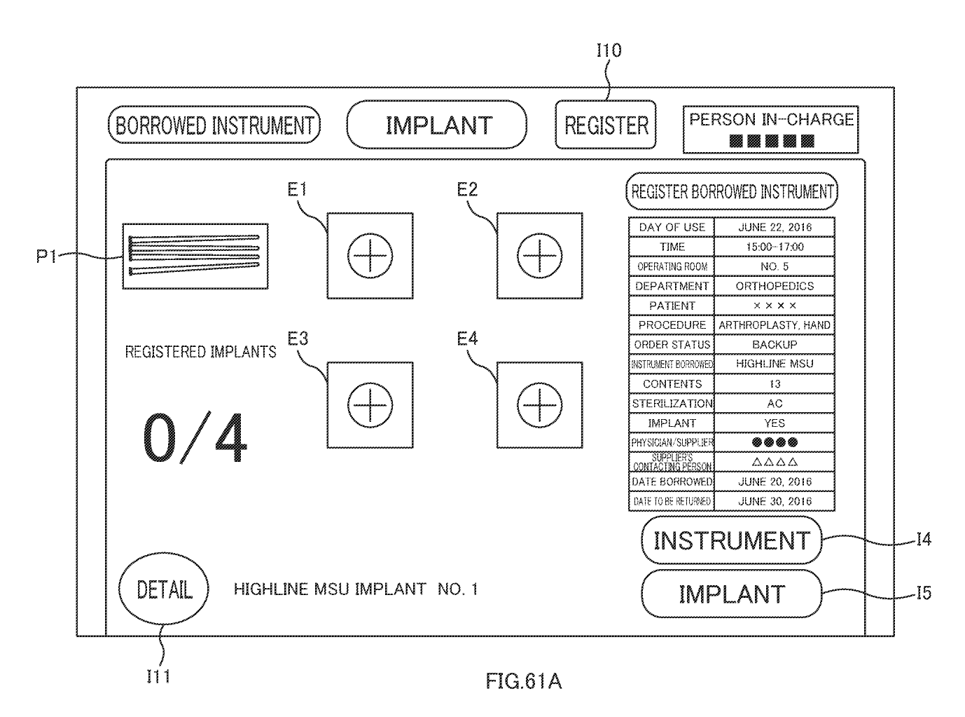

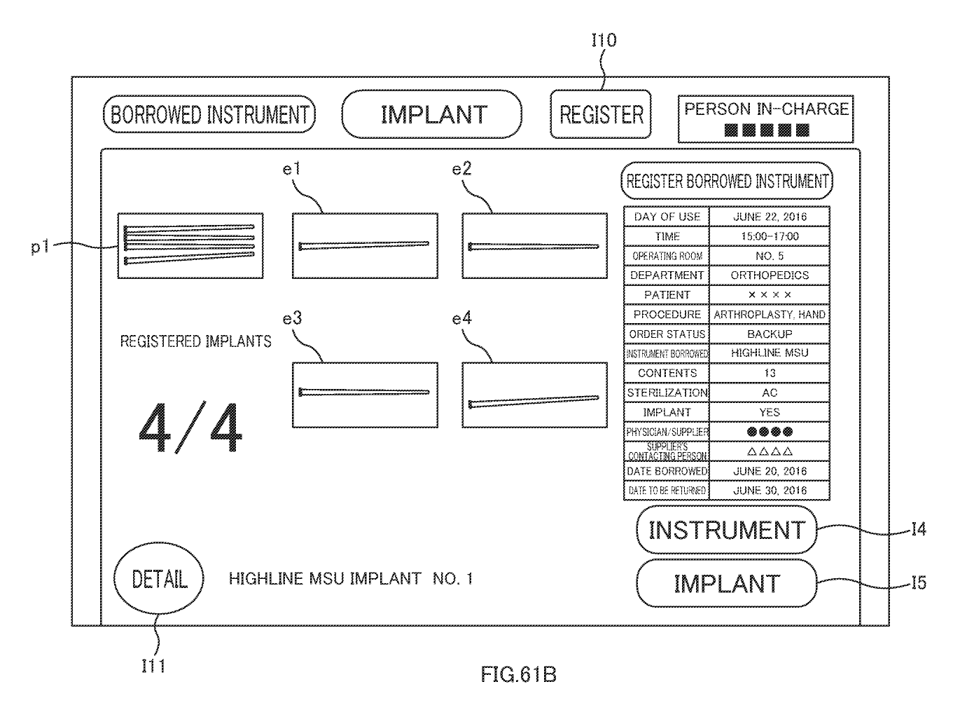

Furthermore, when registering the items of the implant, the operator selects a “detail” icon I 9 in the registration screen shown in FIG. 60B. The first display controller 70a allows the registration screen for the items of the implant p1 to be displayed (see FIG. 61A). In this example, since the information that the implant p1 includes four items has been entered beforehand, in the registration screen, areas E1 to E4 for registering images of the four items are displayed. The operator selects the areas E1 to E4 one after another. In response to the selection, the first display controller 70a allows a folder of images related to the items to be displayed. The operator selects the image of the items included in the implant p1 from the folder. The first display controller 70a allows the images of the selected items to be displayed on the display 40 (see FIG. 61 B). When the operator selects a “register” icon I110, the instrument register 70b registers the images of the items e1 to e4 and the number (4) of the items with association to the implant p1 in the medical instrument storage 60a. The operator can add identification information to the items e1 to e4 by selecting a “detail” icon I11 and entering the size etc. of the items. The instrument register 70b registers the entered identification information with association to the images of the items. FIGS. 61A and 61B are examples of the “second registration screen”.

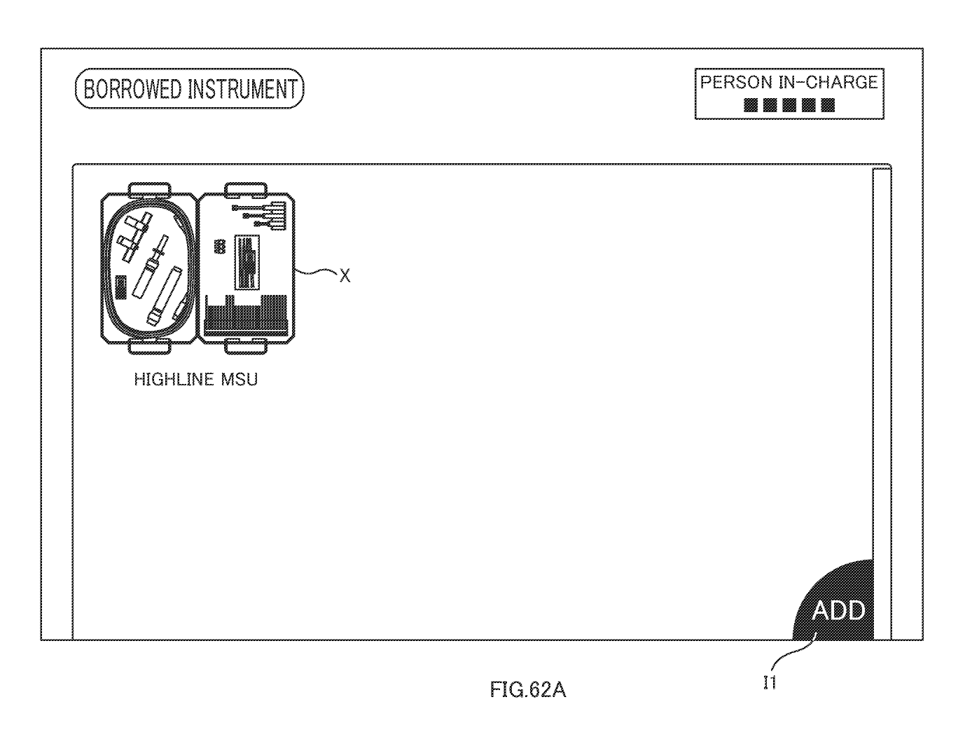

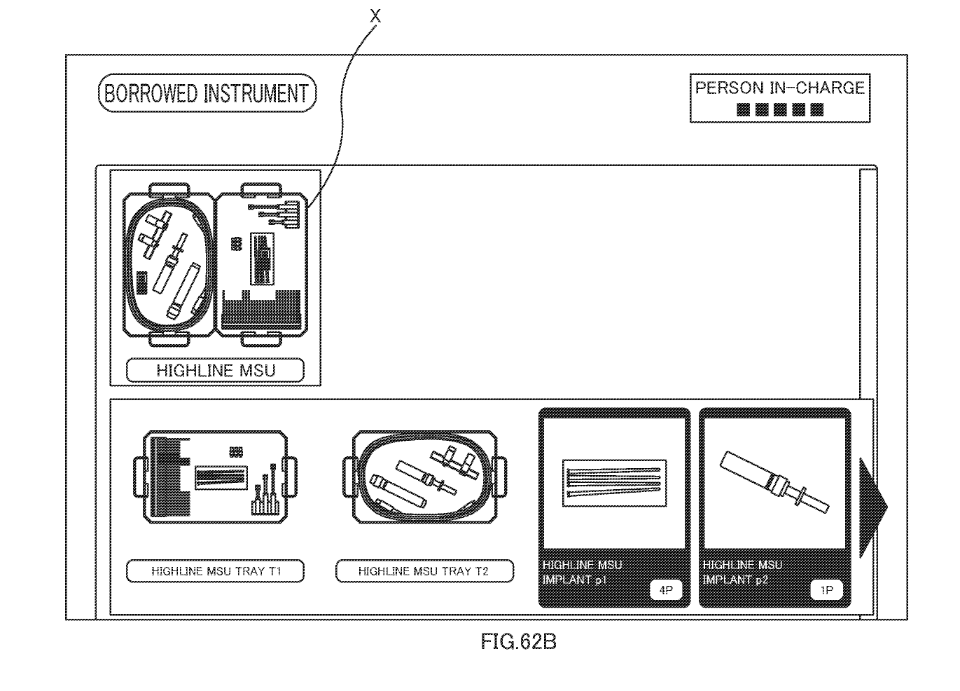

As described above, when the registrations of the borrowed instrument X and the instrument(s) and implant(s) included in the borrowed instrument X are completed as described above, the image of the borrowed instrument X is displayed on the registration screen of FIG. 58A (see FIG. 62A). Here, when the operator selects the image of the borrowed instrument X, the first display controller 70a can allow the image of the instrument or the implant registered for the borrowed instrument X to be displayed as a thumbnail (see FIG. 62B). In this case, it is also possible to change the display mode between the area displaying the image of the instrument and the area displaying the image of the implant (for example, surrounding the implant with a border line of a certain color). Furthermore, when the items included in the image of the implant are registered, the first display controller 70a allows the number of the items to be displayed together with the image of the implant.

After each medical procedure, it is necessary to managed whether the number of the borrowed instruments and the number of the instruments included therein are the same as those before the medical procedure (whether nothing is missing) or whether the implant was used or not. The terminal P2 according to the present preferred embodiment functions as a device for performing counting of borrowed instruments and implants (items) collected after medical procedures. The terminal P2 preferably includes a communicator 80, a storage 110, and a controller 120, and is connected to a display 90 and an operator 100.

The communicator 30 exchange data with the server S and the terminal P1 via the network N. The display 90 displays, for example, images of medical instruments and a list of individual instruments. The display 90 and the terminal P2 may be one unit. The operator 100 is an input interface such as a mouse. Alternatively, the display 90 may be a touch-panel screen that doubles as the operator 100. An operator enters commands to the terminal P2 via the operator 100. The operator 100 and the terminal P2 may be one unit.

The storage 110 is a large-capacity storage that stores various kinds of data. As shown in FIG. 56, in this preferred embodiment, a borrowed instrument storage 110a and a display layout storage 110b are portions of a storage region of the storage 110.

In the present preferred embodiment, each storage stores data that are identical to those stored in the storage of the terminal P1. When the data in the terminal P1 has been changed (for example, a new borrowed instrument has been registered), the terminal P2 receives the changed data indirectly through the server S or directly from the terminal P1 and updates the stored data.

The controller 120 preferably includes a CPU and a memory (which are not shown). The CPU achieves different kinds of control functions by executing an operating program stored in the memory. The memory is a storage that stores a program or programs executed by the CPU or temporarily stores various pieces of information upon execution of the program(s). The controller 120 detects signals (command inputs) from the operator 120 and executes corresponding processing operations.

The controller 120 according to the present preferred embodiment defines and functions as a second display controller 120a and a counter 120b.

The second display controller 120a performs various display controls in the terminal P2.

The second display controller 120a according to the present preferred embodiment allows, after the use of a borrowed instrument, the image of the borrowed instrument to be displayed on the display 90. In addition, the second display controller 120a allows the images of the items related to the borrowed instrument to be displayed on the display 90. The display of these images is described later.

The display of these images is performed based on the layout data stored in the display layout storage 60b. The second display controller 120a is an example of the “display controller.”

The counter 120b performs counting of the borrowed instruments according to a command input. Furthermore, the counter 120b performs counting of the items based on the selection of the images of the items. Details of the counting process are described later.

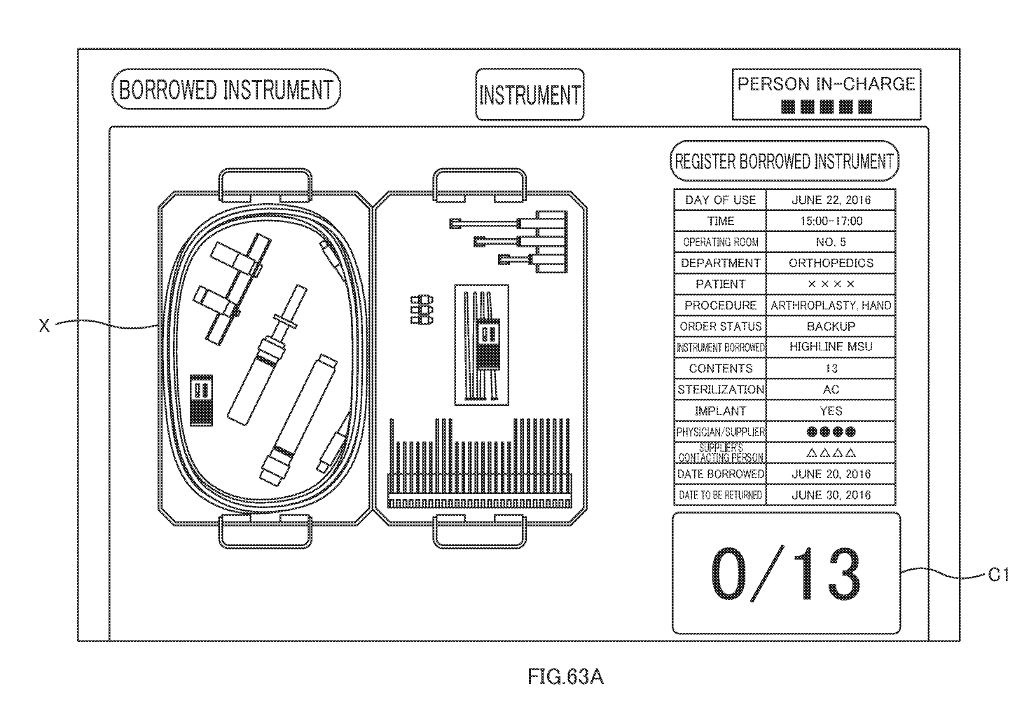

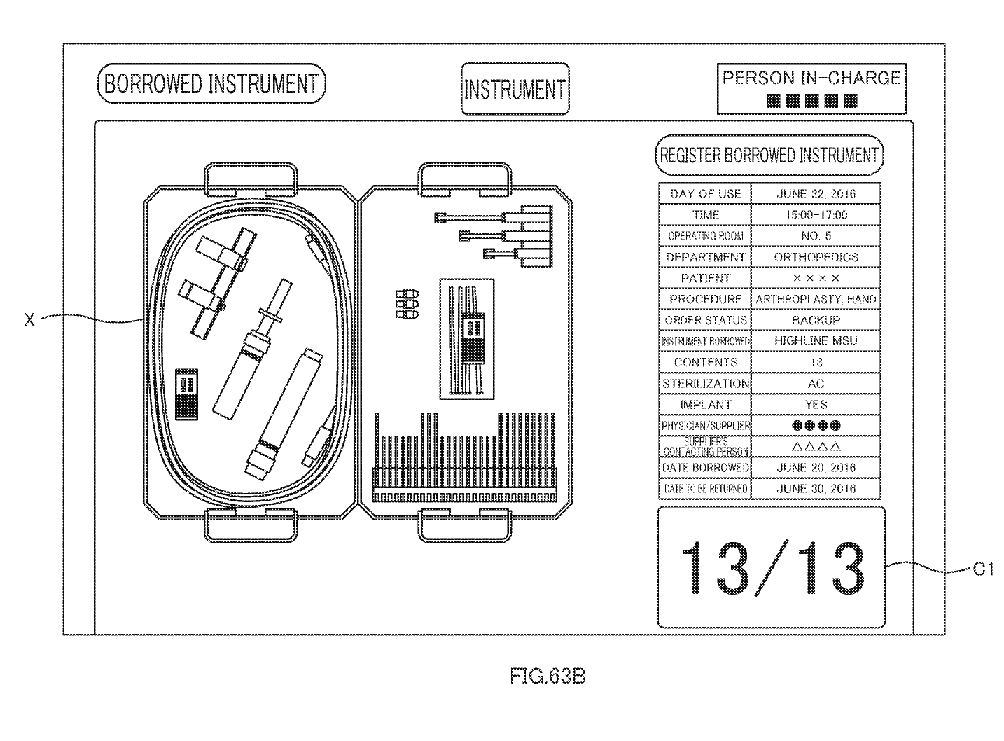

Here, referring to FIGS. 63A to 66B, described is a specific example of the process performed in the terminal P2 from the counting of the instruments included in the borrowed instrument X to their return. FIGS. 63A to 66B show screens displayed on the display 90. It is assumed that the operator enters various inputs (selections) via the operator 100.

When the operator logs in to the system 1 through the terminal P2, the second display controller 120a allows the count screen for the borrowed instrument X to be displayed (see FIG. 63A). On the count screen of FIG. 63A, a counter C1 is displayed. In the counter C1, the number (13) of instruments included in the information regarding the borrowed instrument X is displayed. Here, the operator touches the count screen while checking the number of instruments included in the collected borrowed instrument X kept close at hand. The counter 120b changes the numerical value of the counter C1 according to the number of times of the touch (see FIG. 63B). The counter 120b registers the result of counting in, for example, the borrowed instrument storage 110a with association to the borrowed instrument X. Some of the borrowed instruments have identifiers such as two-dimensional symbols on the manufacturer side. In this case, instead of touching the count screen, counting of the instruments may be performed by reading the identifier with a reader (not shown).

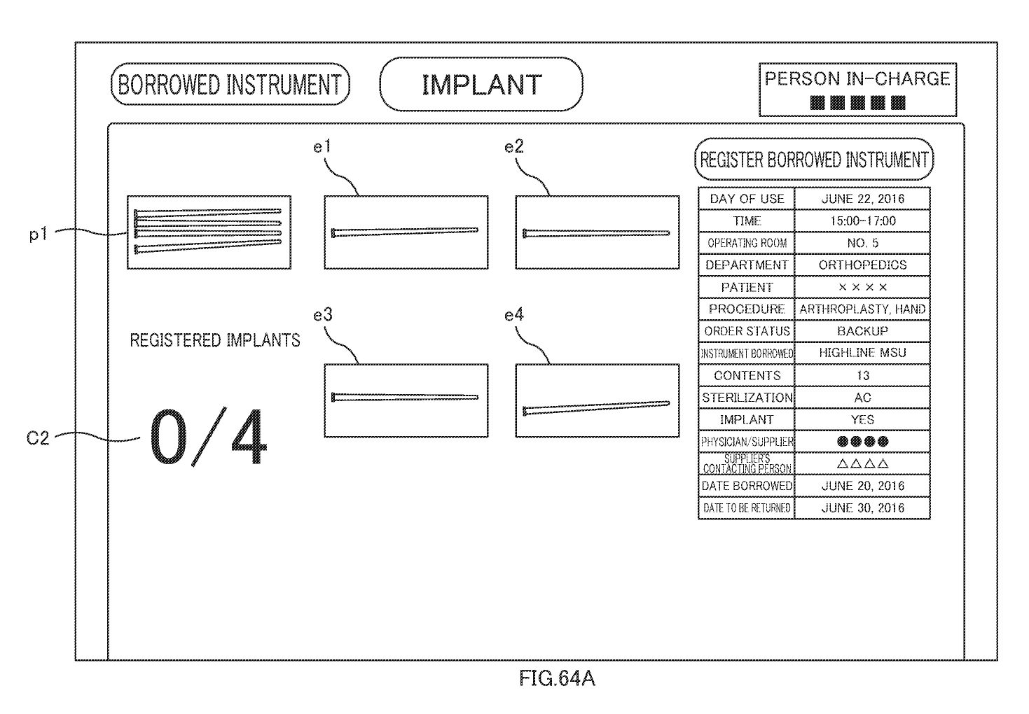

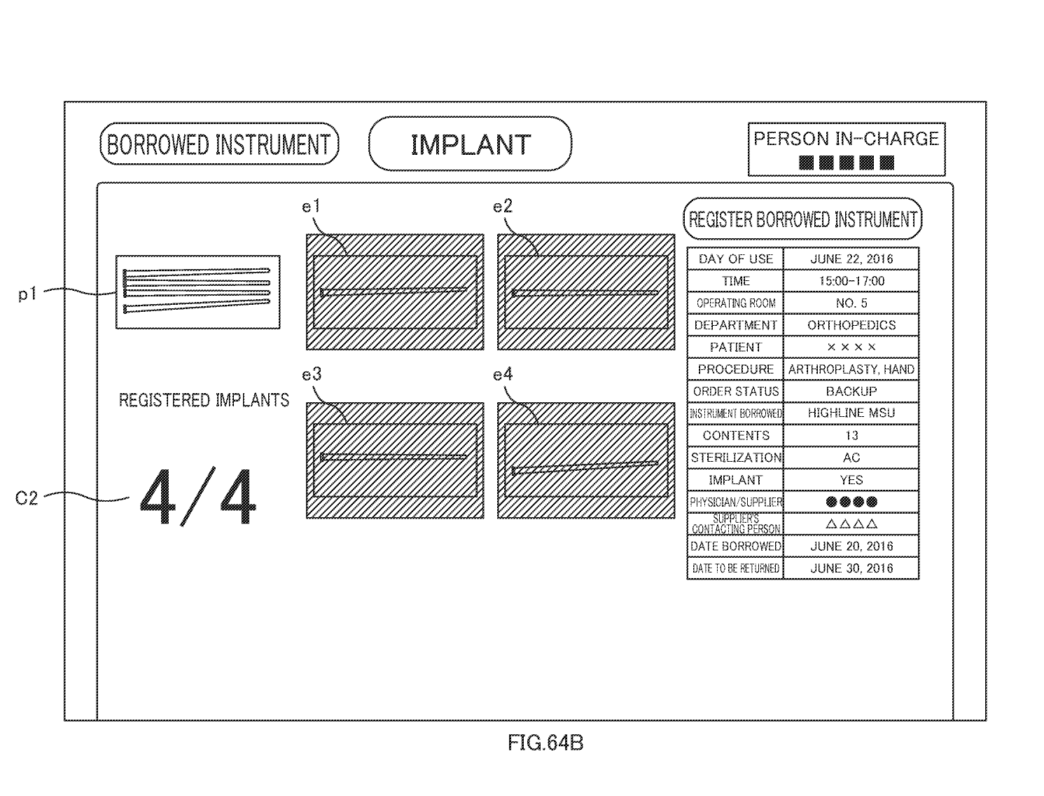

Furthermore, when counting of the items included in the implant p1 is performed, the second display controller 120a allows the count screen for the items to be displayed (see FIG. 64A). On the count screen of FIG. 64A, a counter C2 is displayed. In the counter C2, the number (4) of the items included in the implant p1 is displayed. Here, the operator selects the image of the corresponding item while checking the number of items included in the implant kept close at hand. The counter 120b changes the numerical value of the counter C2 according to the number of times of the selection. Furthermore, the second display controller 120a changes the display mode of the selected image (see FIG. 64 B in which the selected image is indicated by hatching). By changing the display mode in this manner, the operator can easily know which of the items included in the implant was used (or not). The counter 120b registers the result of counting in, for example, the borrowed instrument storage 110a with association to the borrowed instrument X.

In the case where the identification information is associated with an image of an item, the second display controller 120a can allow the identification information to be displayed together with the image of the item in the count screen. Although the example of counting the number of items included in the implant has been described here, it is also possible to count the number of implants using the same method as described above when a plurality of implants are included in the borrowed instrument.

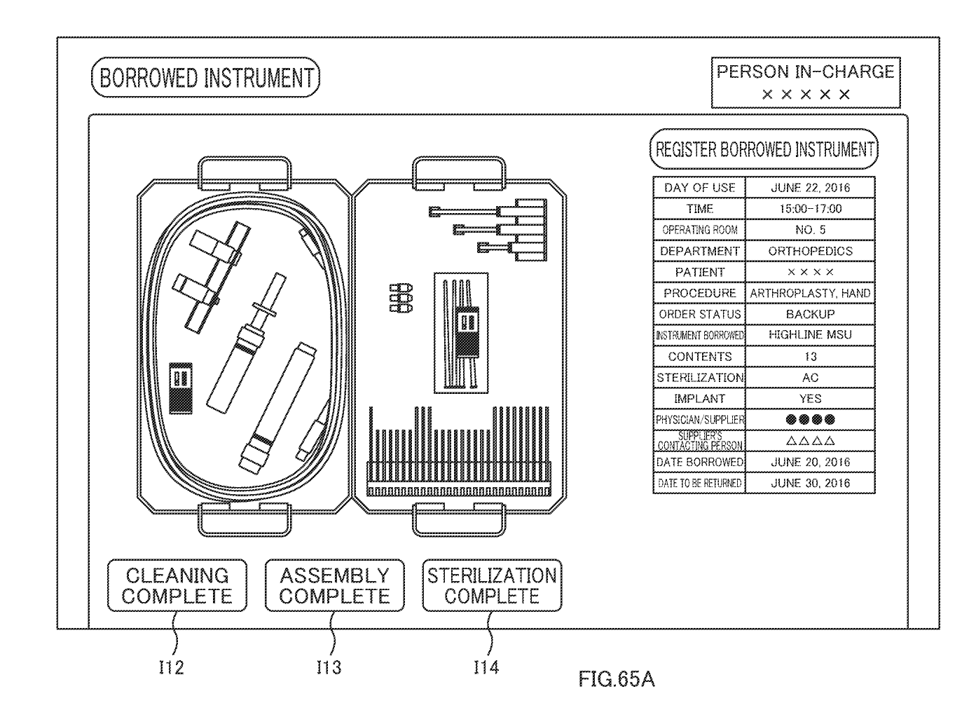

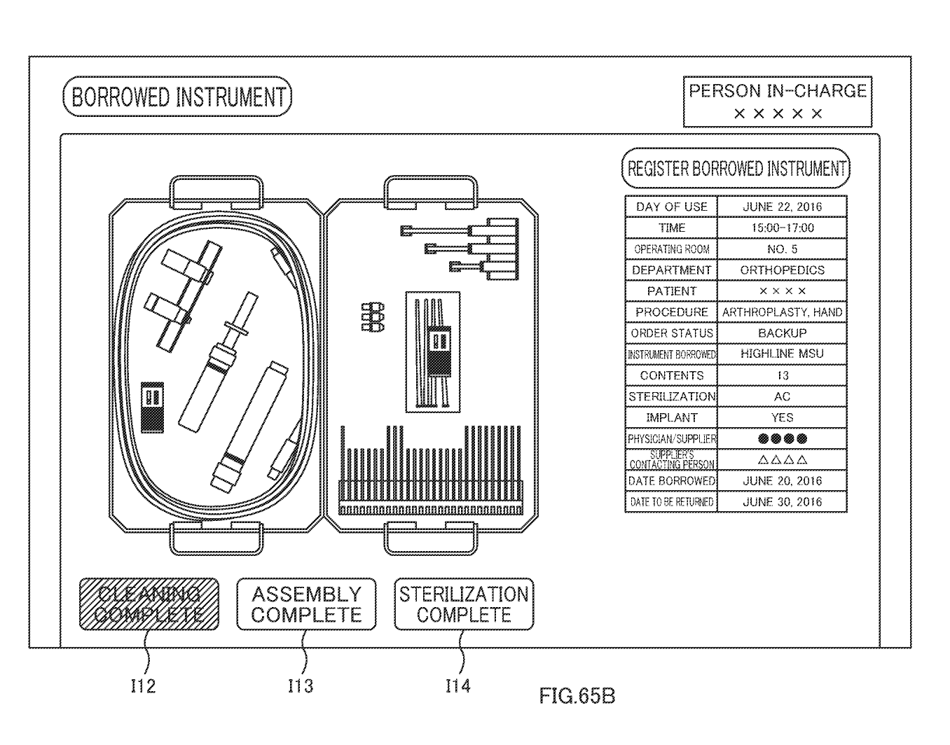

After the quantity of the instruments and the implants included in the borrowed instrument X has been checked, the borrowed instrument X is subjected to cleaning, assembly, and sterilization. When the quantity check is completed, the second display controller 120a allows a “cleaning complete” icon I12, an “assembly complete” icon I13, and a “sterilization complete” icon I14 to be displayed on the screen on which the image or the like of the borrowed instrument X is displayed (see FIG. 65A).

It should be noted that whether or not to display these icons may be identified based on the information supplied in association with the borrowed instruments. For example, in the example of FIG. 58C, sterilization is entered for the borrowed instrument X. Therefore, it can be determined that sterilization is necessary for the borrowed instrument X. In this case, the second display controller 120a allows the “sterilization complete” icon I14 to be displayed based on the information entered.

After the cleaning, assembly and sterilization are completed, the operator selects each icon. The second display controller 120a changes the display mode of the selected icon from the display mode of the unselected icon (see FIG. 65B, in which the selected “cleaning complete” icon I12 is indicated by hatching).

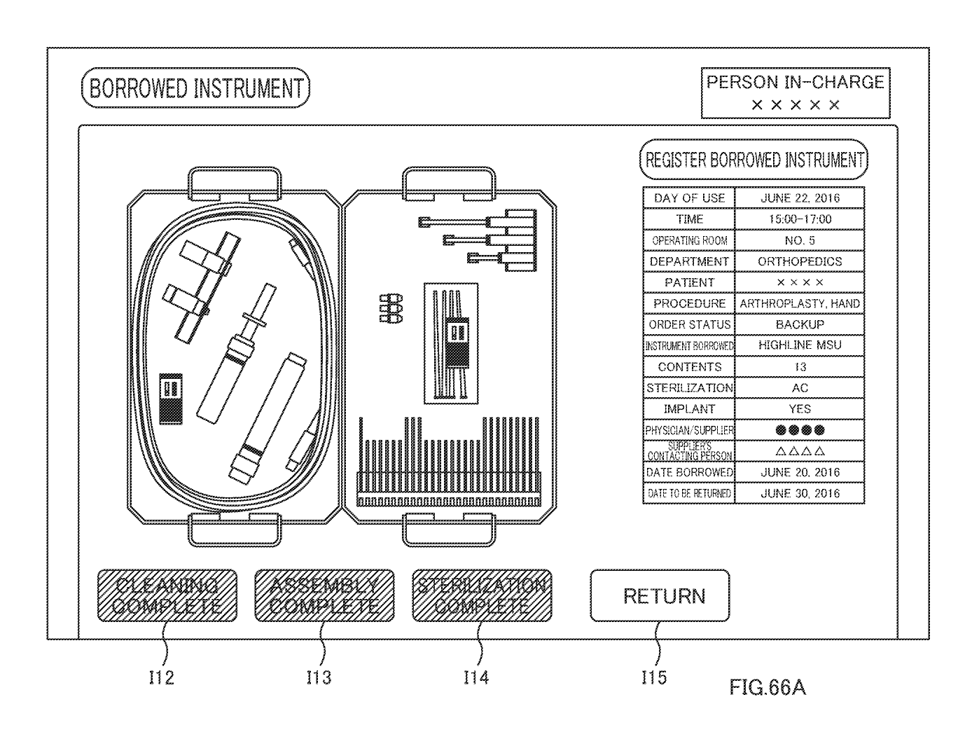

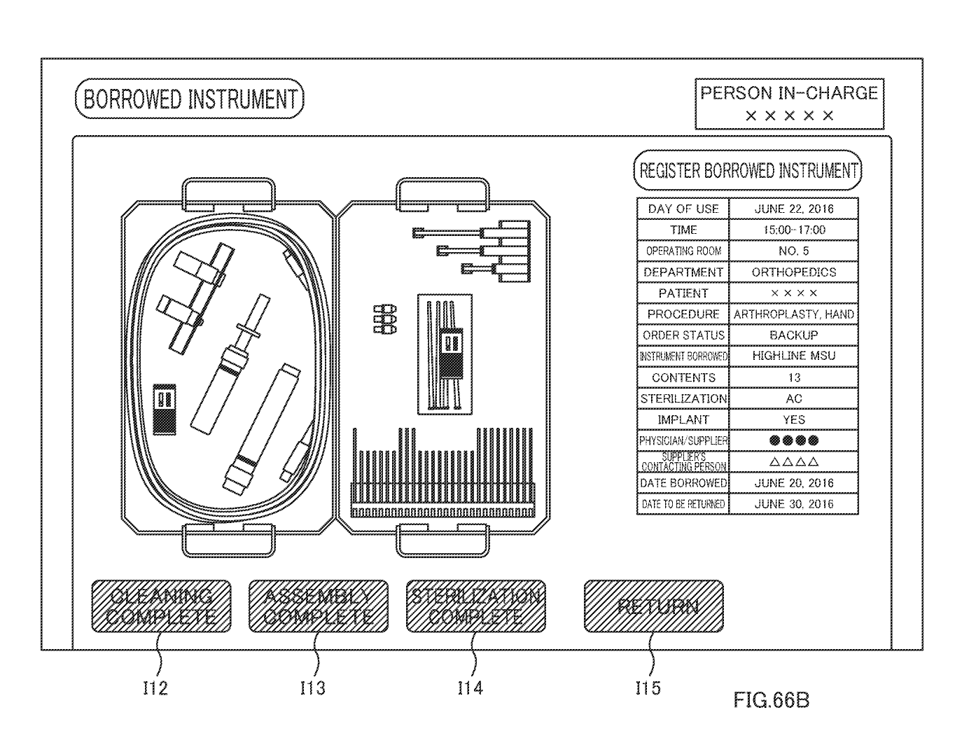

When all the icons displayed in FIG. 65A are selected, the second display controller 120a allows a “return” icon I15 to be displayed (see FIG. 66A). After the return to the manufacturer has been completed, the operator selects the “return” icon I15, whereby the information indicating that the borrowed instrument X has been returned is registered in the borrowed instrument storage 110a.

It should be noted that the information regarding the borrowed instrument X may be deleted from the borrowed instrument storage 110a based on the selection of the “return” icon I15. In addition, the aforementioned cleaning, assembly, sterilization, and return of the borrowed instrument may be performed by a terminal other than the terminal P2 (for example, a terminal located in an area for cleaning).

As described above, according to the system 1 of the present preferred embodiment, it becomes possible to register images of implants included in a new borrowed instrument and images of items included in the implants prior to using the borrowed instrument. Therefore, the operator can accurately manage the information regarding the borrowed instrument and implants. In addition, by performing counting of the instruments and implants collected after the use of the borrowed instrument, it is possible to easily find whether no instrument has been lost or which implant was used. In particular, the number of implants (items included in the implants) is different before and after a medical procedure. However, according to the system 1 of the present preferred embodiment, for example, when one or more implants are missing, it can be determined whether the lack is the result of the use in a medical procedure, or lose during a medical procedure, or where or not they were not present from the beginning at the time of borrowing. That is, according to the system 1 of the present preferred embodiment, it is possible to manage implants included in borrowed instruments.

### Seventh Preferred Embodiment

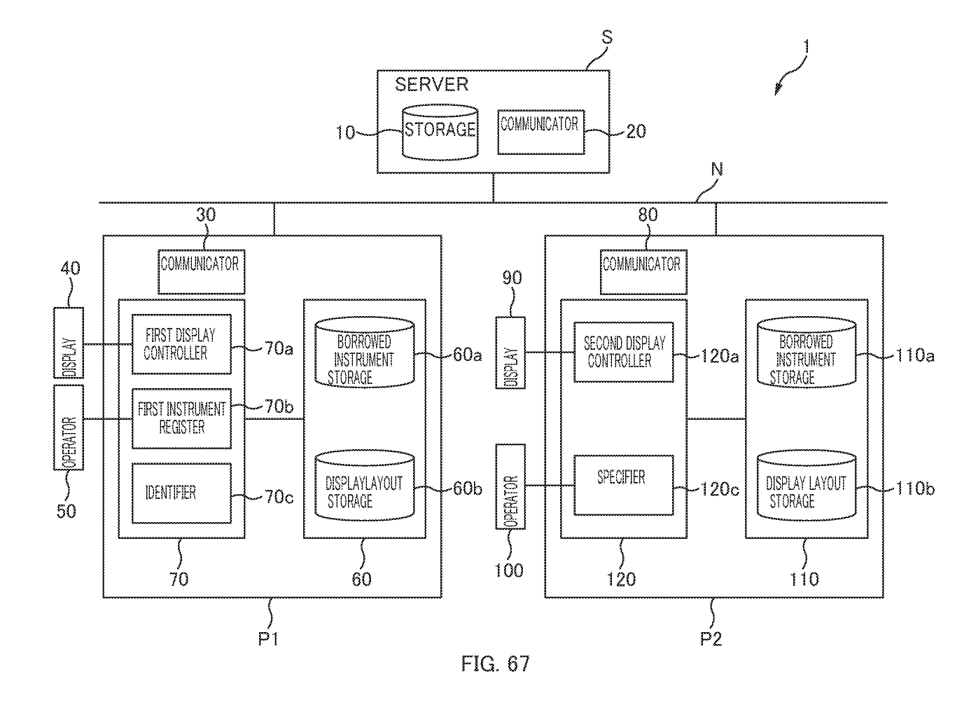

Referring to FIG. 67, an implant management system (hereinafter, also referred to as a “system 1”) according to a seventh preferred embodiment of the present invention is described.

The sixth preferred embodiment has been described with an example in which the items that were actually used are identified by allowing images of items of the pre-registered implant to be displayed on the count screen after the use of the borrowed instrument and visually comparing them with the items close at hand. In this preferred embodiment, an example in which identification of items that were actually used is automatically performed using image processing is described. Detailed descriptions of the components that are the same as those in the sixth preferred embodiment are omitted.

As shown in FIG. 67, the controller 120 of the terminal P2 according to the present preferred embodiment functions as a second display controller 120a and a specifier 120c.

The specifier 120c specifies a used item based on a photographed image of the item that was not used and images of items.

For example, it is assumed that among the items e1 to e4 included in the implant p1, the item e4 was used in a medical procedure (embedded in the patient's body). In this case, only the items e1 to e3 are included as the implant p1 that was collected after the medical procedure. The operator takes pictures of the collected items e1 to e3 using an image-pickup device. The photographed images of the items e1 to e3 are transmitted from the photographing device to the terminal P2. The specifier 120c compares the images of the items e1 to e4 associated with the implant p1 with the photographed images of the items e1 to e3 and determines which image matches. A known image processing method can be used to determine whether or not each image matches. In this case, the images of the items e1 to e3 have their photographed matches. On the other hand, the item e4 does not have any photographed match. Therefore, the specifier 120c can specify the item e4 as the used item.

The second display controller 120a according to the present preferred embodiment allows the image of the specified item to be displayed in a different manner from the images of other items. For example, when the item e4 is specified in the above example, the second display controller 120a allows the image of the item e4 to be displayed on the display 90 using a color that is different from colors of the images of the items e1 to e3.

As described above, according to the system 1 of the present preferred embodiment, it is possible to easily find the items that were used in actual medical procedures from a plurality of similar items included in the implant.

The server S in the above preferred embodiment has a role of interfacing a plurality of terminals (the terminal P1, the terminal P2). Data about the borrowed instrument may be managed only by the server S (the storage 10), and each terminal may be configured not to store the data about any borrowed instrument. In this case, the storage capacity of each terminal can be reduced. Furthermore, an implant management system according to a preferred embodiment of the present invention may be configured with only a plurality of terminals without any server S. Alternatively, it is also possible to configure the implant management system with only one terminal. In this case, the display operator of that one terminal also has the functions of the first display controller 70a and the second display controller 120a.

It is also possible to supply a program to a computer using a non-transitory computer readable medium with an executable program thereon in the above preferred embodiment. Examples of the non-transitory computer readable medium include magnetic storage media (e.g. flexible disks, magnetic tapes, and hard disk drives), CD-ROMs (read only memories).

While preferred embodiments of the present invention have been described above, it is to be understood that variations and modifications will be apparent to those skilled in the art without departing from the scope and spirit of the present invention. The scope of the present invention, therefore, is to be determined solely by the following claims.

1-38. (canceled)

39: A note information management device for medical instruments, the note information management device comprising:
an original data storage that stores original image data for one or more medical instruments;
a note data generator that generates one or more note data, each of the one or more note data representing a note information for a predetermined one of the original image data;
a note data storage that chronologically stores the generated note data with association to the predetermined one of the original image data; and
a display controller that allows a note data selected from the note data that are associated with the predetermined one of the original image data to be displayed on the predetermined one of the original image data.

40: The note information management device according to claim 39, wherein a latest note data among the note data is allowed to be displayed by the display controller.

41: The note information management device according to claim 39, wherein only the predetermined one of the original image data is displayed by the display controller.

42: The note information management device according to claim 39, wherein, when a new note information is entered with the note data being displayed on the predetermined one of the image data, a new note data in which the new note information is merged with the note data is generated by the note data generator; and
the new note data is stored in the note data storage as a data that is different from the note data.

43: The note information management device according to claim 39, wherein the note data includes a stroke data in which the note information is drawn on the predetermined one of the original image data.

44: The note information management device according to claim 39, wherein the one or more medical instruments includes a set of a plurality of medical instruments.

45: A note information management system for medical instruments, the note information management system comprising:
a plurality of note information management devices for medical instruments, each of the plurality of note information management devices including:
an original data storage that stores original image data for one or more of the medical instruments;
a note data generator that generates one or more note data, each of the one or more note data representing a note information entered for a predetermined one of the original image data;
a note data storage that chronologically stores the generated noted data with association to the predetermined one of the original image data; and
a display controller that allows a note data selected from the note data that are associated with the predetermined one of the original image data to be displayed on the predetermined one of the original image data; and
a server including:
a server-side original data storage that stores image data that are identical to the original image data stored in the original data storage;
a server-side note data storage that chronologically stores the note data that are transmitted from one of the note information management devices with association to the predetermined one of the original image data; and
a note data distributor that distributes the note data stored in the server-side note data storage to another of the plurality of note information management devices.

46: The note information management system for medical instruments according to claim 45, wherein
when the image data is allowed to be displayed, the display controller of the another of the plurality of note information management devices makes an inquiry to the server whether there is a note data that is not stored in the note data storage; and
if it is determined by the note data distributor, based on the inquiry, that a note data that is not stored in the note data storage is stored in the server-side note data storage, the note data is distributed to the another of the plurality of note information management devices by the note data distributor.

47: The note information management system for medical instruments according to claim 45, wherein the original data storage of one of the plurality of note information management devices and the original data storage of the another of the plurality of note information management devices store a same image data for one of the medical instruments.

48: A computer-aided management system for medical instruments, the computer-aided management system comprising:
a display operator that allows medical instruments used for a medical procedure to be displayed and, when one of the medical instruments is selected, allows a count screen to be displayed, the count screen being used to perform counting of individual instruments included in the one of the medical instruments; and
a count log generator that performs the counting according to a command input and creates count logs for the medical instruments; wherein
the display operator allows the count logs generated before and after the medical procedure to be displayed.

49: A computer-aided management system for medical instruments, the computer-aided management system comprising:
a display operator that allows medical instruments used for a medical procedure to be displayed and, when one of the medical instruments is selected, allows a count screen to be displayed, the count screen being used to perform counting of individual instruments included in the one of the medical instruments;
a count log generator that performs the counting according to a command input and creates count logs for the medical instruments; and
a comparator that compares the count logs generated before and after the medical procedure.

50: The computer-aided management system for medical instruments according to claim 48, further comprising a medical instrument register that registers the medical instruments used for the medical procedure; wherein
the medical instrument register deletes a part of predetermined ones of the medical instruments used for the medical procedure.

51: The computer-aided management system for medical instruments according to claim 50, wherein the medical instrument register adds a new medical instrument to the predetermined ones of the medical instruments used for the medical procedure.

52: The computer-aided management system for medical instruments according to claim 51, wherein the new medical instrument is capable of being added during the medical procedure, and the count log generator creates a count log for the new medical instrument.

53: The computer-aided management system for medical instruments according to claim 48, wherein the display operator allows a list and/or one or more images of the individual instruments included in the medical instruments to be displayed as the count screen.

54: The computer-aided management system for medical instruments according to claim 53, wherein the counting is performed by the count log generator when a choice of a name of an instrument in the list or a choice of individual images, or an input of an identifier assigned to each of the medical instruments is received.

55: The computer-aided management system for medical instruments according to claim 48, wherein the count log generator adds an information of a user who has performed the counting to the count log.

56: A management system comprising:
an acquirer that acquires a number of unused medical instruments or a number of used medical instruments for each item category for medical instruments; and
a storage that stores the number of unused medical instruments or the number of used medical instruments acquired by the acquirer.

57: A management system comprising:
an adder that acquires a total number of unused medical instruments or a total number of used medical instruments for each item category for medical instruments by summing up numbers of the unused medical instruments or numbers of the used medical instruments; and
a storage that stores the total number of unused medical instruments or the total number of used medical instruments acquired by the adder.

58: The management system according to claim 57, further comprising:
a total-number-of-transfers adder that acquires a total number of transferred medical instruments by summing up the numbers of transferred medical instruments for each item category for medical instruments; and
a total-number-of-transfers storage that stores the total number of transferred medical instruments acquired by the total-number-of-transfers adder.

59: The management system according to claim 58, further comprising:
a calculator that calculates a ratio between the total number of transferred medical instruments that have been stored in the total-number-of-transfers storage and the total number of unused medical instruments or the total number of used medical instruments that have been stored by storage.

60: An implant management system comprising:
an identifier that identifies whether a borrowed instrument includes an implant based on an input of information regarding the borrowed instrument that is a medical instrument borrowed from an outside;
a display controller that allows, when the borrowed instrument includes the implant, a registration screen to be displayed, the registration screen being used to register the implant; and
an instrument register that registers an image of the implant selected on the registration screen with association to the borrowed instrument that includes the implant.

61: The implant management system according to claim 60, wherein
the implant includes two or more items; and
the instrument register registers an image of the two or more items and a number of the two or more items with association to the borrowed instrument that includes the implant.

62: The implant management system according to claim 61, further comprising:
a counter that performs counting of the borrowed instrument according to a command input; wherein
the display controller allows the image of the two or more items related to the borrowed instrument to be displayed after the use of the borrowed instrument; and
the counter is incremented for the two or more items based on a choice of the image of the two or more items.

63: The implant management system according to claim 62, wherein the instrument register registers an identification information to identify the items with association to the image of the items; and
the display controller allows the identification information to be displayed along with the image of the two or more items.

64: The implant management system according to claim 63, further comprising:
a specifier that specifies a used item based on a photographed image obtained by photographing the item that has not been used and the image of the two or more items; wherein
the display controller allows the image of the specified item and an image of other items to be displayed in a different mode.

65: The implant management system according to claim 60, wherein the instrument register registers a user information of a user who has performed the registration of the implant or the items with association to the image of the implant.

66: An implant management system comprising:
a display controller that allows a first registration screen used to register a borrowed instrument to be displayed, the borrowed instrument being a medical instrument borrowed from an outside;
an instrument register that registers the borrowed instrument; and
an identifier that determines whether the borrowed instrument includes an implant based on an input of information regarding the borrowed instrument; wherein
the display controller allows, when the borrowed instrument includes the implant, a second registration screen to be displayed, the second registration screen being used to register the implant;
the instrument register registers an image of the implant on the second registration screen with association to the borrowed instrument that includes the implant.
